(19) United States
(12) Patent Application Publication
(10) Pub. No.: US 2016/0113900 A1
(43) Pub. Date: Apr. 28, 2016

(54) INHIBITORS OF MITOCHONDRIAL STAT3 AND USES THEREOF IN MODULATION OF MAST CELL EXOCYTOSIS

(71) Applicant: Yissum Research Development Company of the Hebrew University of Jerusalem Ltd, Jerusalem (IL)

(72) Inventors: Ehud RAZIN, Mevaseret Zion (IL); Zohar YAGIL, Beit Zait (IL); Tal Hadad ERLICH, Jerusalem (IL)

(21) Appl. No.: 14/626,941

(22) Filed: Feb. 20, 2015

Related U.S. Application Data
(60) Provisional application No. 62/067,483, filed on Oct. 23, 2014.

(51) Int. Cl. A61K 31/381; A61K 9/00; A61K 9/127

(52) U.S. Cl. CPC A61K 31/381; A61K 9/127; A61K 9/007

(57) ABSTRACT

The invention provides composition of matter comprising at least one STAT3 inhibiting moiety or any vehicle, matrix, nano- or micro-particle comprising the same, associated with at least one mitochondrial targeting moiety. The invention further provides compositions comprising said mitochondrial-targeted STAT3 inhibitor, methods and uses thereof in inhibiting mast cell degranulation and in treating disorders induced by activation of MC.

### FIELD OF THE INVENTION

The invention relates to modulators of Mast Cell (MC) exocytosis. More particularly, the invention provides mitochondrial-targeted STAT3 inhibitors, methods and uses thereof in inhibiting mast cell degranulation and in treating disorders induced by activation of MC.

### BACKGROUND REFERENCES

1. Malaviya R, Ikeda T, Ross E, Abraham S N. Mast cell modulation of neutrophil influx and bacterial clearance at sites of infection through TNF-alpha. Nature 1996; 381:77-80.
2. Echtenacher B, Mannel D N, Hultner L. Critical protective role of mast cells in a model of acute septic peritonitis. Nature 1996; 381:75-7.
3. Matsuda H, Watanabe N, Kiso Y, Hirota S, Ushio H, Kannan Y, et al. Necessity of IgE antibodies and mast cells for manifestation of resistance against larval Haemaphysalis longicornis ticks in mice. J Immunol 1990; 144:259-62.
4. Lantz C S, Boesiger J, Song C H, Mach N, Kobayashi T, Mulligan R C, et al. Role for interleukin-3 in mast-cell and basophil development and in immunity to parasites. Nature 1998; 392:90-3.
5. Pevec B, Pevec M R, Markovic A S, Batista I. [The high-affinity IgE-receptor signaling pathway in the treatment of allergies]. Acta Med Croatica 2011; 65:425-34.
6. Tagen M, Elorza A, Kempuraj D, Boucher W, Kepley C L, Shirihai O S, et al. Mitochondrial uncoupling protein 2 inhibits mast cell activation and reduces histamine content. J Immunol 2009; 183:6313-9.
7. Zhang B, Alysandratos K D, Angelidou A, Asadi S, Sismanopoulos N, Delivanis D A, et al. Human mast cell degranulation and preformed TNF secretion require mitochondrial translocation to exocytosis sites: relevance to atopic dermatitis. J Allergy Clin Immunol 2011; 127:1522-31 e8.
8. Zhang B, Weng Z, Sismanopoulos N, Asadi S, Therianou A, Alysandratos K D, et al. Mitochondria Distinguish Granule-Stored from de novo Synthesized Tumor Necrosis Factor Secretion in Human Mast Cells. Int Arch Allergy Immunol 2012; 159:23-32.
9. Takei M, Endo K. Histamine release and calcium concentrations in rat mast cells are dependent on intracellular ATP: effects of prostaglandin D2. Prostaglandins Leukot Essent Fatty Acids 1994; 50:357-62.
10. Kitahata Y, Nunomura S, Terui T, Ra C. Prolonged culture of mast cells with high-glucose medium enhances the Fc epsilon RI-mediated degranulation response and leukotriene C4 production. Int Arch Allergy Immunol 2010; 152 Suppl 1:22-31.
11. Nagai K, Fukushima T, Oike H, Kobori M. High glucose increases the expression of proinflammatory cytokines and secretion of TNFalpha and beta-hexosaminidase in human mast cells. Eur J Pharmacol 2012; 687:39-45.
12. Gough D J, Corlett A, Schlessinger K, Wegrzyn J, Lamer A C, Levy D E. Mitochondrial STAT3 supports Ras-dependent oncogenic transformation. Science 2009; 324:1713-6.
13. Potla R, Koeck T, Wegrzyn J, Cherukuri S, Shimoda K, Baker D P, et al. Tyk2 tyrosine kinase expression is required for the maintenance of mitochondrial respiration in primary pro-B lymphocytes. Mol Cell Biol 2006; 26:8562-71.
14. Jeong K, Kwon H, Min C, Pak Y. Modulation of the caveolin-3 localization to caveolae and STAT3 to mitochondria by catecholamine-induced cardiac hypertrophy in H9c2 cardiomyoblasts. Exp Mol Med 2009; 41:226-35.
15. Sonnenblick A, Levy C, Razin E. Interplay between MITF, PIAS3, and STAT3 in mast cells and melanocytes. Mol Cell Biol 2004; 24:10584-92.
16. Chung J, Uchida E, Grammer T C, Blenis J. STAT3 serine phosphorylation by ERK-dependent and -independent pathways negatively modulates its tyrosine phosphorylation. Mol Cell Biol 1997; 17:6508-16.
17. Kuroki M, O'Flaherty J T. Extracellular signal-regulated protein kinase (ERK)-dependent and ERK-independent pathways target STAT3 on serine-727 in human neutrophils stimulated by chemotactic factors and cytokines. Biochem J 1999; 341 (Pt 3):691-6.
18. Lee H K, Jung J, Lee S H, Seo S Y, Suh D J, Park H T. Extracellular Signal-regulated Kinase Activation Is Required for Serine 727 Phosphorylation of STAT3 in Schwann Cells in vitro and in vivo. Korean Journal of Physiology & Pharmacology 2009; 13:161-8.
19. Suzuki H, Takei M, Yanagida M, Nakahata T, Kawakami T, Fukamachi H. Early and late events in Fc epsilon RI signal transduction in human cultured mast cells. J Immunol 1997; 159:5881-8.
20. Mor A, Shefler I, Salamon P, Kloog Y, Mekori Y A. Characterization of ERK activation in human mast cells stimulated by contact with T cells. Inflammation 2010; 33:119-25.
21. Baines C P, Zhang J, Wang G W, Zheng Y T, Xiu J X, Cardwell E M, et al. Mitochondrial PKCepsilon and MAPK form signaling modules in the murine heart: enhanced mitochondrial PKCepsilon-MAPK interactions and differential MAPK activation in PKCepsilon-induced cardioprotection. Circ Res 2002; 90:390-7.
22. Alonso M, Melani M, Converso D, Jaitovich A, Paz C, Carreras M C, et al. Mitochondrial extracellular signal-regulated kinases 1/2 (ERK1/2) are modulated during brain development. J Neurochem 2004; 89:248-56.
23. Zhu J H, Guo F, Shelburne J, Watkins S, Chu C T. Localization of phosphorylated ERK/MAP kinases to mitochondria and autophagosomes in Lewy body diseases. Brain Pathol 2003; 13:473-81.
24. Monick M M, Powers L S, Barrett C W, Hinde S, Ashare A, Groskreutz D J, et al. Constitutive ERK MAPK activity regulates macrophage ATP production and mitochondrial integrity. J Immunol 2008; 180:7485-96.
25. Sonnenblick A, Levy C, Razin E. Immunological trigger of mast cells by monomeric IgE: effect on microphthalmia transcription factor, STAT3 network of interactions. J Immunol 2005; 175:1450-5.
26. Levy C, Nechushtan H, Razin E. A new role for the STAT3 inhibitor, PIAS3: a repressor of microphthalmia transcription factor. J Biol Chem 2002; 277:1962-6.
27. Jang H D, Yoon K, Shin Y J, Kim J, Lee S Y. PIAS3 suppresses NF-kappaB-mediated transcription by interacting with the p65/RelA subunit. J Biol Chem 2004; 279:24873-80.
28. Long J, Wang G, Matsuura I, He D, Liu F. Activation of Smad transcriptional activity by protein inhibitor of activated STAT3 (PIAS3). Proc Natl Acad Sci USA 2004; 101:99-104.
29. Kotaja N, Aittomaki S, Silvennoinen O, Palvimo J J, Janne O A. ARIP3 (androgen receptor-interacting protein 3) and other PIAS (protein inhibitor of activated STAT) proteins differ in their ability to modulate steroid receptor-dependent transcriptional activation. Molecular Endocrinology 2000; 14:1986-2000.
30. Roise D, Schatz G. Mitochondrial presequences. J Biol Chem 1988; 263:4509-11.
31. Omura T. Mitochondria-targeting sequence, a multi-role sorting sequence recognized at all steps of protein import into mitochondria. J Biochem 1998; 123:1010-6.
32. Smith R A, Porteous C M, Gane A M, Murphy M P. Delivery of bioactive molecules to mitochondria in vivo. Proc Natl Acad Sci USA 2003; 100:5407-12.
33. Quintana A, Schwindling C, Wenning A S, Becherer U, Rettig J, Schwarz E C, et al. T cell activation requires mitochondrial translocation to the immunological synapse. Proc Natl Acad Sci USA 2007; 104:14418-23.
34. Sena L A, Li S, Jairaman A, Prakriya M, Ezponda T, Hildeman D A, et al. Mitochondria Are Required for Antigen-Specific T Cell Activation through Reactive Oxygen Species Signaling. Immunity 2013; 38:225-36.
35. Yagil Z, Nechushtan H, Kay G, Yang C M, Kemeny D M, Razin E. The enigma of the role of protein inhibitor of activated STAT3 (PIAS3) in the immune response. Trends Immunol 2010; 31:199-204.
36. Venkatesha R T, Berla Thangam E, Zaidi A K, Ali H. Distinct regulation of C3a-induced MCP-1/CCL2 and RANTES/CCL5 production in human mast cells by extracellular signal regulated kinase and PI3 kinase. Mol Immunol 2005; 42:581-7.
37. Zhang S, Cherwinski H, Sedgwick J D, Phillips J H. Molecular mechanisms of CD200 inhibition of mast cell activation. J Immunol 2004; 173:6786-93.
38. Duan W, Chan J H, Wong C H, Leung B P, Wong W S. Anti-inflammatory effects of mitogen-activated protein kinase kinase inhibitor U0126 in an asthma mouse model. J Immunol 2004; 172:7053-9.
39. Kimata M, Inagaki N, Kato T, Miura T, Serizawa I, Nagai H. Roles of mitogen-activated protein kinase pathways for mediator release from human cultured mast cells. Biochem Pharmacol 2000; 60:589-94.
40. Liao B C, Hou R C, Wang J S, Jeng K C. Enhancement of the release of inflammatory mediators by substance P in rat basophilic leukemia RBL-2H3 cells. J Biomed Sci 2006; 13:613-9.
41. McMurray J S. A new small-molecule Stat3 inhibitor. Chem Biol 2006; 13:1123-4.
42. Schust J, Sperl B, Hollis A, Mayer T U, Berg T. Stattic: a small-molecule inhibitor of STAT3 activation and dimerization. Chem Biol 2006; 13:1235-42.
43. Redell M S, Ruiz M J, Alonzo T A, Gerbing R B, Tweardy D J. Stat3 signaling in acute myeloid leukemia: ligand-dependent and -independent activation and induction of apoptosis by a novel small-molecule Stat3 inhibitor. Blood 2011; 117:5701-9.
44. Pan J, Quintas-Cardama A, Kantarjian H M, Akin C, Manshouri T, Lamb P, et al. EXEL-0862, a novel tyrosine kinase inhibitor, induces apoptosis in vitro and ex vivo in human mast cells expressing the KIT D816V mutation. Blood 2007; 109:315-22.
45. Siegel A M, Stone K D, Cruse G, Lawrence M G, Olivera A, Jung M Y, et al. Diminished allergic disease in patients with STAT3 mutations reveals a role for STAT3 signaling in mast cell degranulation. J Allergy Clin Immunol 2013; 132:1388-96 e3.
46. Belostotsky R, Lorberboum-Galski H. Apoptosis-inducing human-origin Fcepsilon-Bak chimeric proteins for targeted elimination of mast cells and basophils: a new approach for allergy treatment. J Immunol 2001; 167:4719-28.
47. Fishman A, Prus D, Belostotsky R, Lorberboum-Galski H. Targeted Fc2′-3-PE40 chimeric protein abolishes passive cutaneous anaphylaxis in mice. Clin Exp Immunol 2000; 119:398-403.
48. Belostotsky R, Lorberboum-Galski H. Utilizing Fcepsilon-Bak chimeric protein for studying IgE-FcepsilonRI interactions. Clin Immunol 2004; 110:89-99.
49. Reddy Cheruka Apoorva, et al., PLOS 2014; 9(3):1-11.sPELA
50. Zupancic Spela et al., Mol. Pharmaceuitics 2014; 11:2334-2345.

### BACKGROUND OF THE INVENTION

Mast cells are primary effectors of the allergic inflammatory response and of the immune response to many pathogens including parasites¹⁻⁴. Their immunological activation is carried out via their surface high affinity receptor for IgE (FcεRI). Cross-linking of IgE by multivalent antigens leads to the aggregation of FcεRI, inducing a signaling cascade followed by exocytosis⁵.

The role played by mitochondria in mast cell activation has recently been described⁶. In this work it was observed that over expression of mitochondrial uncoupling protein 2 (UCP2) inhibited mast cell activation and reduced histamine release. Moreover, studies in a human mast cell line showed that degranulation and TNFα secretion require mitochondrial translocation into the exocytosis sites^{7, 8}.

Adequate ATP levels in mast cells have been shown to be essential for degranulation and cytokine secretion⁹⁻¹¹. It was observed that depletion of glucose from mast cell cultured medium resulted in a rapid decrease in ATP levels followed by a reduction in histamine secretion whereas supplementation of glucose to the medium induced a time-dependent increase in ATP which correlated with the recovery in histamine secretion⁹. It was found in another study that prolonged culture of mast cells at high glucose levels enhanced IgE-Ag mediated degranulation and leukotriene C4 production¹⁰. In a similar study, enhanced TNF-α secretion following immunological activation of mast cells was observed when there were high glucose levels in the culture medium¹¹. However, the role played by mitochondrial ATP produced by oxidative phosphorylation (OXPHOS) during mast cell degranulation remains elusive and to the inventor's knowledge has not yet been reported in the literature.

It has been recently shown in a variety of cell types that signal transducer and activator of transcription factor 3 (STAT3) has a non-canonical role in mitochondria whereby it regulates OXPHOS by modulating mitochondrial electron transport chain (ETC) activity¹²⁻¹⁴. STAT3 null cells were shown to have 50% less total ATP content, decreased activity of electron transfer chain complexes and exhibited a higher mitochondrial membrane potential¹²⁻¹⁴. Mitochondrial STAT3 function required serine 727 phosphorylation¹². Interestingly, the inventors have previously reported that STAT3 undergoes phosphorylation on serine 727 during mast cell activation¹⁵; however, the significance of this phosphorylation is not fully understood.

The extracellular signal-regulated kinases (ERK) are known to phosphorylate STAT3 on the serine 727 residue in a variety of cells¹⁶⁻¹⁸ and to be activated after IgE-Ag stimuli^{19,20}. However, ERK expression in the mitochondria has just begun to be explored. ERK was found in the mitochondria of cardiomyocytes, where it plays a role in protection from ischemia reperfusion²¹. In neuronal cells, ERK1/2 were found in the outer membrane/intermembrane space^{22,23}. Furthermore in macrophages U0126, an ERK pathway inhibitor, was shown to inhibit ATP production²⁴. Thus, in the present work the correlation and interplay between mitochondrial ERK mediated STAT3 phosphorylation and OXPHOS in activated mast cells, has been explored. Phosphorylation of STAT3 on serine 727 after mast cell activation is followed by its association with the protein inhibitor of activated STAT3 (PIAS3) which inhibits its transcriptional activity.^{15, 25} PIAS3, the main inhibitor of STAT3, is a multifunctional protein that plays a significant role in the modulation of additional key transcription factors such as MITF,²⁶ NFkB,²⁷ SMADs²⁸ and estrogen receptor.²⁹ Therefore, the present invention aimed at determining whether the inhibition of STAT3 activity by PIAS3 also occurs in the mitochondria, with subsequent effect on OXPHOS.

Reddy et al.,⁴⁹ disclose mitochondrial-targeted curcuminoids and uses thereof in inducing apoptosis in breast cancer cells. Zupancic et al.,⁵⁰ disclose mitochondrial targeted DQAsomes for curcumin inhalation (curcumin loaded vesicles formed from amphiphile dequalinium).

Allergies are currently managed by using medications such as epinephrine, antihistamines, anti-inflammatory medications (corticosteroids), decongestants, leukotriene inhibitors, that target the mediators released by the MC activated cells. However, effective therapeutic approaches that specifically target the activation of mast cells thereby interrupting and preventing the release of such mediators are not available. Therefore, there is an urge need for a therapeutic approach allowing immediate treatment for all the allergy symptoms related to mast cell mediators.

The present invention explores the potential involvement of STAT3 in mitochondrial ATP production and in exocytosis of immunologically activated mast cells both in vitro and in vivo and show that mitochondrial STAT3 serves as a key regulator of mast cell exocytosis. As such, the invention provides specific and targeted attenuation of mast cell activation and of the associated disorders.

### SUMMARY OF THE INVENTION

In a first aspect, the invention relates to a composition of matter comprising at least one STAT3 inhibiting moiety or any vehicle, matrix, nano- or micro-particle comprising the same, associated with at least one mitochondrial targeting moiety.

A further aspect of the invention relates to a pharmaceutical composition comprising a pharmaceutically acceptable carrier and, as an active ingredient, at least one STAT3 inhibiting moiety or any vehicle, matrix, nano- or micro-particle comprising the same, associated with at least one mitochondrial targeting moiety, or any composition of matter comprising the same as described by the invention. In more specific embodiments, the active ingredient may be present in an amount effective for inhibiting and/or decreasing mast cell (MC) degranulation, while retaining STAT3 nuclear function's.

In yet a further aspect, the invention provides a method for inhibiting, reducing or decreasing MC activation, the method comprising contacting mast cells with an effective amount of at last one STAT3 inhibiting moiety or any vehicle, matrix, nano- or micro-particle comprising the same, associated with at least one mitochondrial targeting moiety, or with any composition comprising the same.

In another aspect, the invention provides a method for treating a subject suffering from a medical condition associated with or induced by mast cell activation. In more specific embodiments, the method of the invention may comprise administering to the subject a therapeutically effective amount of at last one STAT3 inhibiting moiety or any vehicle, matrix, nano- or micro-particle comprising the same, associated with at least one mitochondrial targeting moiety, or with any composition comprising the same according to the invention.

### BRIEF DESCRIPTION OF THE FIGURES

FIG. 1A-1B. Degranulation and ATP levels of immunologically activated RBL cells grown in glucose-free medium

FIG. 1A. RBL cells were sensitized with 100 ng/ml IgE for 2 h, followed by 30 min stimulation with DNP-BSA and then analyzed for β-hexosamindase release. Results represent mean±SEM, n=3, *p<0.05.

FIG. 1B. ATP levels of RBL cells grown in glucose-free medium for 24 h. Cells were sensitized with 100 ng/ml IgE for 2 h and then challenged for 5 min with DNP-BSA, lysed and then analyzed for total cell ATP. Results represent mean±SEM, n=3, *p<0.05.

FIG. 2A-2D. Effect of Stattic on mitochondrial characteristics

FIG. 2A. BMMC and RBL cells were incubated with 60 μM of the STAT3 inhibitor Stattic for 20 min. Cells were lysed and then total cell ATP was measured. Results represent mean±SEM, n=3, *p<0.05.

FIG. 2B. Oxygen consumption analysis. RBL cells were grown in 24-well plates, treated as for A and then intracellular oxygen consumption was determined using the Seahorse Extracellular Flux (XF) Analyzer. Results represent mean±SEM, n=3, *p<0.05.

FIG. 2C. ETC activity analysis. RBL cells were incubated with 60 μM Stattic for 30 min and then analyzed for ETC complex activity. CS-Cytrate synthase, SDH-Succinate dehydrogenase, II+III-Complex 2 and 3, Cox-Cytochrome c oxidase. Results represent mean±SEM, n=3, *P<0.05.

FIG. 2D. RBL cells were sensitized with 100 ng/ml IgE for 2 h, followed by 5 min DNP-BSA challenge. 20 min before the addition of DNP-BSA, 60 μM of the STAT3 inhibitor Stattic was added to the medium. The levels of P-STAT3 727 (pS727-STAT3) and actin control in the cell lysates were determined by Western blot analysis. One representative out of 3 independent experiments is shown.

FIG. 3. Densitometry quantification of STAT3 serine 727 phosphorylation inhibition by Stattic

The protein bands were quantified in Image J software and β-actin was used as the internal control. Results represent mean±SEM, n=3, *p<0.05.

FIG. 4: Effect of Stattic on apoptosis in activated RBL cells

RBL cells were sensitized with 100 ng/ml IgE for 2 h, followed by 30 min DNP-BSA challenge. 20 min before the addition of DNP-BSA, 60 μM Stattic was added to the medium. The levels of caspase3 were determined by Western blot analysis. One representative out of 3 independent experiments is shown.

FIG. 5A-5D. Effect of Stattic on mast cell degranulation

FIGS. 5A-5B. BMMC and RBL cells were sensitized with 100 ng/ml IgE for 2 h, followed by 30 min DNP-BSA challenge. 20 min before the addition of DNP-BSA, 60 μM Stattic was added to the medium. β-hexosamindase release and TNF-α secretion were then measured. Results represent mean±SEM, n=3, *p<0.05.

FIGS. 5C-5D. BMMCs were sensitized with 100 ng/ml IgE for 2 h, followed by 30 min DNP-BSA challenge. 20 min before the addition of DNP-BSA, 60 μM Stattic was added to the medium. 3-hexosamindase release, and TNF-α and Granzyme B secretion were then measured. Results represent mean±SEM, n=3, *p<0.05.

FIG. 6. Effect of Stattic on RBL degranulation

RBL cells were sensitized with 100 ng/ml IgE for 2 h, followed by 30 min DNP-BSA challenge. 20 min before the addition of DNP-BSA, the stated concentrations of the STAT3 inhibitor Stattic were added to the medium. 3-hexosamindase release was then measured. Results represent mean±SEM, n=3, *p<0.05.

FIG. 7. Effect of C188-9 on RBL degranulation

RBL cells were sensitized with 100 ng/ml IgE for 2 h, followed by 30 min DNP-BSA challenge. 10 min before the addition of DNP-BSA, 250 μM of the STAT3 inhibitor C188-9 was added to the medium. B-hexosamindase release was then measured. Results represent mean±SEM, n=3, *p<0.05.

FIG. 8. Effect of Curcumin on RBL degranulation

RBL cells were sensitized with 100 ng/ml IgE for 2 h, followed by 30 min DNP-BSA challenge. 10 min before the addition of DNP-BSA, the stated concentrations of the STAT3 inhibitor Curcumin were added to the medium. β-hexosamindase release was then measured. Results represent mean, n=3.

FIG. 9A-9C. Mitochondrial STAT3 and ERK signaling in mast cell activation

FIG. 9A. RBL cells were sensitized with 100 ng/ml IgE for 2 h, followed by 5 min DNP-BSA challenge. The cytosolic and mitochondrial levels of phospho-STAT3 serine 727 (pS727-STAT3), STAT3, ERK1/2, caspase3, BCL2 and caspase3 were determined by Western blot analysis. One representative out of 3 independent experiments is shown.

FIG. 9B. RBL cells were sensitized with 100 ng/ml IgE for 2 h, followed by 5 min DNP-BSA challenge. 20 minutes before the addition of DNP-BSA, 40 μM U0126 was added to the medium. The levels of phospho-STAT3 727 (pS727-STAT3) and actin as control in the cell lysates were determined by Western blot analysis. One representative out of 3 independent experiments is shown.

FIG. 9C. RBL cells were treated as for B. The cells were fractionated into cytosol and mitochondria. The levels of phospho-STAT3 serine 727 (pS727-STAT3), caspase3 and Cytochrome C were determined by Western blot analysis. One representative out of 3 independent experiments is shown.

FIG. 10A-10D. Densitometry quantification of mitochondrial STAT3 serine 727 ERK dependent phosphorylation during RBL activation

The protein bands were quantified in Image J software. FIG. 10A. Mitochondrial STAT serine 727 phosphorylation; FIG. 10B. Mitochondrial ERK expression; FIG. 10C. Total STAT serine 727 phosphorylation; FIG. 10D. Mitochondrial STAT serine 727 phosphorylation.

FIG. 11A-11C. Effect of U0126 on mitochondrial characteristics

FIG. 11A. RBL cells were sensitized with 100 ng/ml IgE for 2 h, followed by 30 min DNP-BSA challenge. 20 minutes before the addition of DNP-BSA, 40 μM U0126 was added to the medium. 3-hexosamindase release was then measured. Results represent mean±SEM, n=3, *p<0.05.

FIG. 11B. RBL cells were sensitized with 100 ng/ml IgE for 2 h, followed by 5 min DNP-BSA challenge. 20 minutes before the addition of DNP-BSA, 40 μM U0126 was added to the medium and then the cells were lysed and analyzed for total cell ATP. Results represent mean±SEM, n=3, *p<0.05.

FIG. 11C. Oxygen consumption analysis. RBL cells were grown in 24-well plates, treated as for B and then intracellular oxygen consumption was determined using the Seahorse XF Analyzer. Results represent mean±SEM, n=4, *p<0.05.

FIG. 12. Effect of U0126 on RBL degranulation

RBL cells were sensitized with 100 ng/ml IgE for 2 h, followed by 30 min DNP-BSA challenge. 20 min before the addition of DNP-BSA, the stated concentrations of the U0126 were added to the medium. β-hexosamindase release was then measured. Results represent mean±SEM, n=3, *p<0.05.

FIG. 13. Effect of U0126 on apoptosis in activated RBL cells RBL cells were sensitized with 100 ng/ml IgE for 2 h, followed by 5 min DNP-BSA challenge. 20 min before the addition of DNP-BSA, 40 μM U0126 was added to the medium. The levels of caspase3 were determined by Western blot analysis. One representative out of 3 independent experiments is shown.

FIG. 14A-14F. Effect of PIAS3 on mitochondrial characteristics

FIG. 14A. PIAS3 mitochondrial localization analysis—RBL cells were fractionated into cytosol and mitochondria. Mitoplasts were prepared by digitonin treatment. The levels of PIAS3, caspase3, pyruvate dehydrogenase (PDH) and cytochrome C were determined by Western blot analysis. One representative out of 3 independent experiments is shown.

FIG. 14B. PIAS3 mitochondrial translocation quantification—RBL cells were activated (or not) with IgE, challenged with DNP-BSA for specific times, and then stained for PIAS3 by anti-PIAS3 antibody followed by dylight 650-conjugated anti-rabbit, together with Hoechst staining to visualize nuclei and mitotracker red to visualize mitochondria. Mitochondrial translocation was evaluated by ImageStream flow cytometry. For each treatment, 10,000 cells were collected. Positive single cells were gated, and mitochondrial expression of PIAS3 was plotted. In order to reduce unspecific plotting the nuclei staining area was omitted. Representative images of each treatment are shown.

FIG. 14C. ATP quantification analysis, RBL cells were transfected with empty plasmid control or MTS-PIAS3 plasmid. Cells were sensitized with 100 ng/ml IgE for 2 h and then challenged with DNP-BSA for 5 min No significant change between empty plasmid and PIAS3-mito treatments were observed before activation (data not shown). Results represent mean±SEM, n=5, *p<0.05.

FIG. 14D. Oxygen consumption rate analysis, RBL cells were transfected with empty plasmid control or MTS-PIAS3 plasmid. Cells were sensitized with 100 ng/ml IgE for 2 h and then challenged with DNP-BSA and oxygen consumption was determined using the Seahorse XF Analyzer. Results represent mean±SEM, n=4, *p<0.05.

FIG. 14E. Degranulation assay of RBL cells transfected with empty plasmid control or PIAS3-mito plasmid (Relative to empty plasmid). Cells were sensitized with 100 ng/ml IgE for 2 h, followed by 30 min challenge with DNP-BSA and analyzed for 3-hexosamindase release. Results represent mean±SEM, n=3, *p<0.05.

FIG. 14F. Degranulation assay of BMMCs transfected with non-relevant (NR) or with PIAS3 siRNA. Cells were sensitized with 100 ng/ml IgE for 2 h, followed by 30 min challenge with DNP-BSA and analyzed for 3-hexosamindase release. Results represent mean±SEM, n=3, *p<0.05.

FIG. 15. Effect of PIAS3 on RBL degranulation

Degranulation assay of RBL cells transfected with non-relevant or with PIAS3 siRNA. Cells were sensitized with 100 ng/ml IgE for 2 h, followed by 30 min challenge with DNP-BSA and β-hexosamindase release measured. Results represent mean±SEM, n=3, *p<0.05.

FIG. 16. Effect of Stattic on CBMC

CBMC were sensitized with 0.3 μg/ml human myeloma IgE. Five min before the addition of 5 μg/ml rabbit α-human-IgE, 60 μM Stattic was added to the medium. 3-hexosamindase release was then measured. Results represent mean±SEM, n=3, *p<0.05.

FIG. 17. In vivo allergic response assay

Mice were sensitized with IgE anti-DNP through injection to the tail vein. Twenty-four hours later, mice were injected with corn oil or Stattic I.P for 3.5 hours and then challenged with DNP-BSA for 1.5 minutes. Plasma was collected, and histamine levels were measured by using competitive ELISA. Results represent mean±SEM, n=8, *p<0.05.

FIG. 18A-18B. Effect of Mitocur-1/3 on the STAT3 target gene c-myc and apoptosis

FIG. 18A. RBL cells were incubated with the stated concentrations of Mitocur-1/3 for 3 h. The levels of c-myc and cleaved caspase3 in the cell lysates were determined by Western blot analysis. One representative out of 3 independent experiments is shown.

FIG. 18B. RBL cells were incubated with the stated concentrations of Mitocur-1/3 for 24 h. . The levels of β-actin and cleaved caspase3 in the cell lysates were determined by Western blot analysis. One representative out of 2 independent experiments is shown.

FIG. 19A-19G. Time and concentration dependency of Mitocur-1/3's effect on degranulation in RBL cells

FIG. 19A. RBL cells were sensitized with 100 ng/ml IgE for 2 h, followed by 30 min DNP-BSA challenge. 10 min before the addition of DNP-BSA, the stated concentrations of curcumin were added to the medium. β-hexosamindase release was then measured. Results represent mean±S.E. (n=3).

FIG. 19B-19E. RBL cells were sensitized with 100 ng/ml IgE for 10 min (FIG. 19B), 40 min (FIG. 19C), 2 h (FIG. 19D), 3 h (FIG. 19E) or 24 h (FIG. 19F), followed by 30 min DNP-BSA challenge. 10 min before the addition of DNP-BSA, the stated concentrations of Mitocur-1/3 were added to the medium. 3-hexosamindase release was then measured. Results represent one-three experiments.

FIG. 19G. RBL cells were sensitized with 100 ng/ml IgE for 24 h followed by 30 min DNP-BSA challenge. 10 min before the addition of DNP-BSA, the stated concentrations of Mitocur-1/3 were added to the medium. TNF-α secretion was then measured. Results represent mean±S.E. (n=3).

FIG. 20A-20B. Effect of Mitocur-1 and 3 on ATP levels and oxygen consumption

FIG. 20A. RBL cells were incubated for 3 h with Mitocur-1/3 and the ATP levels (pmol/min) were measured.

FIG. 20A. RBL cells were incubated for 3 h with Mitocur-1/3 and the oxygen consumption levels (pmol/min) were measured.

FIG. 21A-21E. Effect of Mitocur-1/3 on mast cell exocytosis

FIGS. 21A-21B. BMMC were sensitized with 100 ng/ml IgE for 2 h, followed by 30 min DNP-BSA challenge. 3 h (FIG. 21A) or 24 h (FIG. 21B) before the addition of DNP-BSA, 6 μM of Mitocur-1 or Mitocur-3 were added to the medium. 3-hexosamindase release. Results represent mean±S.E. (n=3).

FIG. 21C. BMMc were treated as for A TNF-α and IL-6 secretion were then measured. Results represent one experiment.

FIG. 21D-21E. Western blot analysis of cleaved caspase3 levels in BMMC treated as for A&B respectively.

FIG. 22A-22C. In vivo allergic response assay

FIGS. 22A-22B. Mice were sensitized with IgE anti-DNP through injection to the tail vein. Twenty-four hours later, mice were injected with Mitocur-1 (40 mg/ml, FIG. 22A) or Mitocur-3 (80 mg/ml, FIG. 22B) I.P for 3.5 hours and then challenged with DNP-BSA for 1.5 minutes. Plasma was collected, and histamine levels were measured by using competitive ELISA. Results represent mean±SEM, n=5, *p<0.05.

FIG. 22C. shows the effect of Mitocur-3 on human mast cell exicytosis. CBMC were sensitized with 100 ng/ml IgE for 3 days, followed by 30 min DNP-BSA challenge. 3 h before the addition of DNP-BSA 12 μM of Mitocur-3 were added to the medium. β-hexosamindase release was then measured. Results represent mean±SEM, n=3, *p<0.05.

### DETAILED DESCRIPTION OF THE INVENTION

The mitochondrion plays a critical role in the function of immune derived cells as T cell activation requires mitochondrial translocation to the immunological synapse³³ and complex 3 activity was found to be essential for their activation³⁴. Recently it has also been shown that human mast cells require mitochondrial translocation to exocytosis sites for degranulation and TNF secretion^{7, 8}. However, the metabolic mitochondrial pathway involved in mast cell exocytosis is for the first time delineated in the present work. Although ATP has been shown to be essential for mast cell function° ⁻¹¹, its source, whether it is a result of glycolysis or of mitochondrial OXPHOS, or possibly a combination of the two, has not been investigated. Furthermore, the effect of immunological activation on ATP was previously unclear as ATP levels were measured only before immunological activation¹⁰. In the present study the inventors demonstrated the pivotal role of mast cell mitochondria in ATP production during their immunological activation. This was based on the observation that mast cell degranulation occurred even without glycolysis. Moreover, the findings of the present invention that mitochondrial OXPHOS measured both by ATP production and by oxygen consumption were enhanced by immunological activation strongly supported the inventor's hypothesis that mitochondrial ATP plays a key role in this physiological process. The combination of the data presented by the invention indicates an active signaling pathway which regulates mitochondrial ATP production for the initiation of mast cell exocytosis.

STAT3 is an essential protein in the immune system³⁵. The present invention determined that mitochondrial STAT3 serine 727 phosphorylation occurs in mast cells during immunological activation. It is important to note that this phosphorylation was not due to STAT3 translocation, since the basal levels of mitochondrial STAT3 before and after activation were identical. Of all the serine kinases capable of phosphorylating STAT3, ERK1/2 is the most well-characterized protein expressed in the mitochondria²¹⁻²³. Furthermore, the results of the present invention demonstrate ERK1/2 as the exclusive kinase of STAT3 serine 727 in mast cells, as a significant decrease in STAT3 727 serine phosphorylation was observed in U0126 treated cells.

The present invention shows for the first time that ERK1/2 translocate to the mitochondria during mast cell activation. Interestingly, although U0126 is a well-established inhibitor of mast cell function³⁶⁻⁴⁰, the possibility that it functions via an effect on mitochondrial activity has not yet been reported. The findings that mitochondrial OXPHOS measured both by ATP production and by oxygen consumption were decreased by U0126 treatment, strongly supports the inventors hypothesis that the inhibitory effect of U0126 on degranulation could be mediated through mitochondrial OXPHOS inhibition. The decreased mitochondrial STAT3 serine 727 phosphorylation seen during mast cell activation in cells treated with U0126, suggests the mitochondrion as a target of U0126. Thus, the FcεRI mediated MAPK pathway emerges as a regulator of mitochondrial ATP production during the initiation of mast cell exocytosis and connects a signaling pathway to a metabolic pathway.

The initial hypothesis of the present invention was that mitochondrial STAT3 activity is the major mediator of ATP production by OXPHOS during mast cell activation. To prove this, Stattic, a small molecule selective inhibitor of STAT3^{41, 42} and an additional STAT3 inhibitor C188-9⁴³ were used, in a time frame that did not permit nuclear influence and evaluated its effect on mitochondrial activity and on mast cell function. In support of the hypothesis, Stattic severely impaired mitochondrial function as both ATP production and oxygen consumption decreased significantly. This interference in mitochondrial activity was mediated through inhibition of complex 3 activity, which correlates with the effect of STAT3 inhibition¹³. Therefore, STAT3 serine 727 phosphorylation in mast cells could serve as a metabolic switch for the mitochondria to initiate ATP production.

After observing the effect of STAT3 inhibition on mitochondrial activity, the inventors hypothesized that STAT3 inhibition would affect mast cell function. Based on the inventors observation that mast cell degranulation and cytokine secretion were completely abolished both by Stattic and by C188-9 treatment, mitochondrial STAT3 emerges as a key mediator of the onset of exocytosis. Inducing apoptosis in mast cells has been previously proposed as a possible treatment for mast cell disorders such as systemic mastocytosis, possibly through STAT3 inhibition⁴⁴. It is important to emphasize that the effect shown by the invention on exocytosis is not a result of apoptosis as no caspase3 cleavage was observed. The present invention clearly shows that the activity of STAT3 in the mitochondria and not the nuclear activity of STAT3 is important for FcεR signaling which leads to degranulation and cytokine secretion. This is a result of STAT3 mitochondrial activity which regulates energy levels. The inventors base said conclusion on the finding that treatment with a STAT3 inhibitor for minutes and not hours or days can abolish mast cell function with no connection to apoptosis or the nuclear activity of STAT3 as transcription/translation takes more than minutes. It should be appreciated that this surprising rapid effect has clear application in MC exocytosis and associated disorders, where immediate inhibition of such activation is crucial. Furthermore, this effect is selective to STAT3 inhibition as the use of a different STAT3 inhibitor reproduced the degranulation results. In addition, the fact that STAT3 depletion inhibited both degranulation and cytokine secretion clearly indicates that this is a general phenomenon of mast cell exocytosis.

Having established the importance of mitochondrial STAT3 in exocytosis in RBL cells, the inventors found that this role extends to human mast cells since degranulation in IgE activated human CBMC was abolished by Stattic. Since this suggests that STAT3 could be a new target for treating allergy, the inventors investigated STAT3 inhibition in vivo. A marked decrease in histamine secretion by Stattic-treated mice compared to that seen in control mice was observed when an established acute systemic anaphylaxis model was used. In other words, Stattic reduced the response to allergic stimulation.

Seeking the endogenous terminator of this signaling, the inventors investigated PIAS3 since it is the main endogenous inhibitor of STAT3. PIAS3 expression in the mitochondria has not previously been studied. Notably PIAS3 does not contain a consensus mitochondrial target sequence according to the PIAS3 protein accession number (GenBank: EDL85611.1). However, a computer analysis (Mitoport II-v1.101) found that PIAS3 has a 0.8995 probability out of 1 of being present in the mitochondria. The inventors have now confirmed the presence of PIAS3 within in the mitochondria and also observed an increase in its protein level following mast cell activation. Furthermore, by targeting PIAS3 to the mitochondria, the inventors have shown that mitochondrial ATP production during mast cell immunological activation was abolished. The inventors therefore conclude that PIAS3 serves as a major regulator of mitochondrial function. When PIAS3 turned off the metabolic ATP production switch, a significant decrease in mast cell degranulation followed. Moreover, PIAS3 silencing by siRNA resulted in enhanced degranulation. These complementary findings indicate that manipulation of PIAS3 translocation into the mitochondria could affect the allergic response.

To conclude, mitochondrial STAT3 emerges an important regulator of mast cell function. As such, mitochondrial STAT3 could serve as a potential therapeutic target Inhibiting mitochondrial STAT3 either by small molecules or by PIAS3 down regulates mitochondrial activity which is essential for mast cell function. In a very recent paper, Siegel et al reported on the diminished allergic disease in patients with STAT3 mutations, and concluded that future study is required to identify the proximal target of STAT3 in FcεRI signaling.⁴⁵ The inventors have now identified mitochondrial function as the proximal target of STAT3 in FcεRI signaling. Together these findings indicate that STAT3 has a critical role in mast cell function and therefore could emerge as a new target for the manipulation of allergic diseases.

As noted above, there is a need for new therapeutic approaches for the treatment of diseases and disorders associated with mast cell activation. In particular, a need exists for new therapeutic approaches targeting mast cells in order to modulate the release of inflammatory mediators associated with allergic responses.

As shown by the invention and discussed above, targeted inhibition of the mitochondrial activities of STAT3, while retaining its nuclear activities, significantly and specifically blocks activation of mast cells in response to exposure to allergens and thus demonstrates the feasibility of using this approach for treating and preventing disorders associated with activation of mast cells.

Thus, in a first aspect, the invention relates to a composition of matter comprising at least one signal transducer and activator of transcription factor 3 (STAT3) inhibiting moiety or any vehicle, matrix, nano- or micro-particle comprising the same, associated with at least one mitochondrial targeting moiety.

It should be noted that the term “composition of matter” may refer to a single compound, to a complex of several compounds associated together, or to a unique structure or vehicle such as nano or micro particles, micelles, liposomes or any matrix composed of, comprising or encapsulating the STAT3 inhibiting moiety and the mitochondrial targeting moiety. It should be further noted that the mitochondrial transporting or targeting moiety may be attached directly or indirectly via any linker, matrix or vehicle that will be described in more detail herein after. In certain embodiments, the composition of matter according to the invention may further comprise at least one mast cell (MC) targeting moiety.

Mast cells and basophils are effector cells in IgE-associated immune responses, such as those that contribute to asthma and other allergic diseases. The high affinity receptor for IgE (FcεRI) is expressed mainly on mast cells and basophils. Fusing the above moieties to the Fc portion of the human IgE antibody (Fcε) will target specifically FcεRI and therefore affect only mast cells and basophils⁴⁶⁻⁴⁸. Therefore, the composition of matter according to the invention may further comprise at least one Fc portion of the human IgE antibody (Fcε), or any peptides or fragments thereof. In more specific embodiments, nucleotide 406-813 of the mouse immunoglobulin epsilon heavy chain constant region mRNA Genebank J00476, as also denoted by SEQ. ID NO. 12. In yet another embodiment, such MC targeting moiety rimy comprise two human Fcε sequences. Still further embodiments relate to the use of short variant comprising amino acids 301-437 of human IgE, which corresponds exactly to the mouse sequence. More specifically, such fragment includes the Cε2-Cε3 junction and the Cε3 domain of the Fcε, also referred to herein as short Fcε, fragment Cε3 as denoted by the nucleic acid sequence of SEQ ID NO. 20 and the amino acid sequence of ID NO. 21. In yet another embodiment, an MC targeting moiety may be a long variant comprising amino acids 224-443 of the human IgE that includes the complete Cε2 and Cε3 domains and the Cε3-Cε4 junction, also referred to herein as long Fcε fragment Cε2-Cε4, as denoted by the nucleic acid sequence of SEQ ID NO. 23 and the amino acid sequence of SEQ ID NO. 23.

The composition of matter according to the invention comprises at least one STAT3 inhibitor. It should be noted that STAT3, as referred to herein is a Signal transducer and transcription activator that mediates cellular responses to interleukins, KITLG/SCF and other growth factors. May mediate cellular responses to activated FGFR1, FGFR2, FGFR3 and FGFR4, binds to the interleukin-6 (IL-6)-responsive elements identified in the promoters of various acute-phase protein genes. It should be noted that in certain embodiments, STAT3 as used herein may refer to the human STAT3 isoform 1 as disclosed in NP_644805.1. In more specific embodiments said STAT3 variant or isoform 1 comprises the amino acid sequence as denoted by SEQ ID NO. 1. In further embodiments, said isoform 1 is encoded by the nucleic acid sequence as denoted by GenBank Accession number NM_139276.2. In yet some other embodiments, STAT3 as used herein may refer to the human STAT3 isoform 2 as disclosed in NP_003141.2. In more specific embodiments said STAT3 variant or isoform 2 comprises the amino acid sequence as denoted by SEQ ID NO. 2. In further embodiments, said isoform 2 is encoded by the nucleic acid sequence as denoted by GenBank Accession number NM_003150.3. Still further embodiments relate to STAT3 that may refer to the human STAT3 isoform 3 as disclosed in NP_998827.1. In more specific embodiments said STAT3 variant or isoform 3 comprises the amino acid sequence as denoted by SEQ ID NO. 3. In further embodiments, said isoform 3 is encoded by the nucleic acid sequence as denoted by GenBank Accession number NM_213662.1.

It should be appreciated that the present invention may further refer to the murine STAT3. In more specific embodiments an example for the murine STAT3 may be Mus musculus STAT3 as disclosed by AAQ75419.1. More specifically, said STAT3 may comprise the amino acid sequence as denoted by SEQ ID NO. 4. In more specific embodiments, said STAT3 may be encoded by the nucleic acid sequence as disclosed by GenBank Accession number AY299490.1. In still further embodiments, STAT3 referred to by the invention may be Mus musculus STAT3, isoform 1, as disclosed by NP_998824.1. More specifically, said STAT3 may comprise the amino acid sequence as denoted by SEQ ID NO. 5. In more specific embodiments, said STAT3 isoform 1 may be encoded by the nucleic acid sequence as disclosed by GenBank Accession number NM_213659.2. In yet some further embodiments, STAT3 referred to by the invention may be Mus musculus STAT3 isoform 2 as disclosed by NP_998825.1. More specifically, said STAT3 isoform 2 may comprise the amino acid sequence as denoted by SEQ ID NO. 6. In more specific embodiments, said STAT3 may be encoded by the nucleic acid sequence as disclosed by GenBank Accession number NM_213660.2. In still further embodiments, STAT3 referred to by the invention may be Mus musculus STAT3 isoform 3 as disclosed by NP_035616.1. More specifically, said STAT3 isoform 3 may comprise the amino acid sequence as denoted by SEQ ID NO. 7. In more specific embodiments, said STAT3 may be encoded by the nucleic acid sequence as disclosed by GenBank Accession number NM_011486.4.

As noted above, in some embodiments of the present disclosure, the at least one STAT3 inhibiting moiety is associated with at least one mitochondrial targeting moiety, and optionally to the at least one MC targeting moiety discussed above. As used herein, the term “association” refers to the chemical or physical force which holds two entities together. In some embodiments, the association is via at least one chemical bond, namely the STAT3 inhibiting moiety and the mitochondrial targeting moiety may be held together via at least one chemical bonding. Non-limiting examples of such association interactions involving chemical bonding are ionic bonding, electrostatic bonding, covalent bonding, coordination bonding, complexation, hydrogen bonding, van der Waals bonding, hydrophobicity-hydrophilicity interactions, dipole-dipole interactions, London dispersion force etc. In some embodiments, the association is via covalent bonding. It should be understood to a person skilled in the art that in some cases the associative interactions between two atoms or two chemical entities may involve more than one type of chemical and/or physical interactions. In some further embodiments, the at least one STAT3 inhibiting moiety is associated with at least one mitochondrial targeting moiety to form a complex or conjugate. The association between the least one STAT3 inhibiting moiety and the at least one mitochondrial targeting moiety may be direct. In the context of the present disclosure, the association of the two moieties enables delivery or translocation into the mitochondria together as a single entity. The association may be either direct or via a linker forming a single molecule, aggregate, conjugate or complex formed by electrostatic bonds or hydrogen bonds. The association may use structure/s such as polymeric particle, matrix, micelle, liposome wherein the particle comprises the STAT3 inhibitor and features on its surface the mitochondria targeting moiety. The association may be of the type that is dissociated inside the mitochondria or of the type that maintains its integrity inside the mitochondria. If the association is maintained in the mitochondria the STAT3 inhibitor should be associated in such a way to the other moiety so as to maintain its STAT3 inhibiting properties.

Further and in accordance with the present disclosure, the STAT3 inhibiting moiety is associated with the mitochondrial targeting moiety in any stoichiometric ratio. In some embodiments, the association involves a 1:1 ratio. In some embodiments, one STAT3 inhibiting moiety is associated with at least one, at least two, at least three, at least four, at least five, at least ten, at least hundred, at least thousand and even more, mitochondrial targeting moiety. In some other embodiments, at least one, at least two, at least three, at least four, at least five, at least ten, at least hundred, at least thousand and even more, STAT3 inhibiting moiety is associated with one mitochondrial targeting moiety. In some further embodiments, the association may involve at least one, at least two, at least three, at least four, at least five, at least ten, at least hundred, at least thousand and even more, STAT3 inhibiting moieties may be associated with at least one, at least two, at least three, at least four, at least five, at least ten, at least hundred, at least thousand and even more, mitochondrial targeting moieties. As noted above, the at least one STAT3 inhibitory moiety of the invention is associated with at least one mitochondrial targeting moiety, and optionally with at least one MC targeting moiety. It should be appreciated that the association or conjugation as defined herein above apply also to the optional association with the MC targeting moieties. Moreover, it should be appreciated that the association may be between any of these moieties.

In certain embodiments, the at least one mitochondrial targeting moiety of the composition of matter according to the invention may comprise at least one of a lipohilic cation and/or at least one mitochondrial targeting sequence.

The term “mitochondrial targeting moiety” refers to a moiety that is capable of transporting other moieties associated to it selectively into the mitochondria without significant transport to other intracellular organelles. The moiety may be a nucleic acid based moiety, an amino acid based moiety or a small organic molecule based moiety.

More specifically, a mitochondrial targeting group is a group which is capable of concentrating the composition of matter in the mitochondria of a cell. Thus, following incubation of a cell with a composition of matter comprising one or more mitochondrial targeting groups, the concentration of the composition of matter in the mitochondria will be higher than the concentration of composition of matter in the cytosol.

Particularly preferred mitochondrial targeting groups are groups which are capable of concentrating the STAT3 inhibitor specifically in the mitochondrial matrix of a cell. Thus, a composition of matter according to the invention preferably has a mitochondrial matrix/extramitochondrial accumulation ratio of greater than 2, more preferably greater than 3, more preferably greater than 4, as determined by an assay which comprises the following steps. In certain embodiments, the mitochondrial-targeting moiety may be an amino acid-based moiety, specifically, a peptide. Typically, a mitochondrial targeting peptide contains 4 to 70 amino acids. The amino acids are natural or unnatural amino acids. Amino acids are selected from natural amino acids and diphenylalanine, cyclohexylalanine, hexylalanine, methylated tyrosine, dimethyltyrosine and napthylalanine. Said amino acids may be either the D- or L-enantiomers. Preferred amino acids are basic amino acids and aromatic amino acids. Typical basic amino acids are lysine, arginine and glutamine, preferably lysine and arginine. Typical aromatic amino acids are phenylalanine, diphenylalanine, cyclohexylalanine, hexylalanine, tyrosine, methylated tyrosine, dimethyltyrosine and napthylalanine.

The mitochondrial targeting agent can also be a polypeptide including positively charged amino acids. Thus, some embodiments include PTDs that are cationic or amphipathic. Protein transduction domains (PTD), also known as a cell penetrating peptides (CPP), are polypeptides including positively charged amino acids. Therefore, the mitochondrial targeting agent can be a PTD or a CPP. “Protein Transduction Domain” refers to a polypeptide, polynucleotide, carbohydrate, or organic or inorganic compounds that facilitate traversing a lipid bilayer, micelle, cell membrane, organelle membrane, or vesicle membrane. A PTD attached to the compounds disclosed herein, specifically, the STATS inhibiting moiety of the invention facilitates the molecule traversing membranes, for example going from extracellular space to intracellular space, or cytosol to within an organelle such as the mitochondria. PTDs are known in the art, and include but are not limited to small regions of proteins that are able to cross a cell membrane in a receptor-independent mechanism. Although several of PTDs have been documented, the two most commonly employed PTDs are derived from TAT protein of HIV and Antennapedia transcription factor from Drosophila, whose PTD is known as Penetratin.

PTDs can include a sequence of multiple arginine residues, referred to herein as poly-arginine or poly-ARG. In some embodiments the sequence of arginine residues is consecutive. In other embodiments the sequence of arginine residues is non-consecutive.

In yet further embodiments, mitochondrial targeting agents can include short peptide sequences, for example mitochondrial transporters-synthetic cell-permeable peptides, also known as mitochondria-penetrating peptides (MPPs) that are able to enter mitochondria. MPPs are typically cationic, but also lipophilic; this combination of characteristics facilitates permeation of the hydrophobic mitochondrial membrane. For example, MPPs can include alternating cationic and hydrophobic residues. Some MPPs include delocalized lipophilic cations (DLCs) in the peptide sequence instead of, or in addition to natural cationic amino acids. Other variants can be based on an oligomeric carbohydrate scaffold, for example attaching guanidinium moieties due to their delocalized cationic form.

Mitochondrial targeting agents may also include mitochondrial localization signals or mitochondrial targeting signals. Many mitochondrial proteins are synthesized as cytosolic precursor proteins containing a leader sequence, also known as a pre-sequence, or peptide signal sequence.

Typically, cytosolic chaperones deliver the precursor protein to mitochondrial receptors and the General Import Pore (GIP) (Receptors and GIP are collectively known as Translocase of Outer Membrane or TOM) at the outer membrane. The precursor protein is translocated through TOM, and the intermembrane space by small TIMs to the TIM23 or 22 (Translocase of Inner Membrane) at the inner membrane.

Mitochondrial localization/targeting signals generally have of a leader sequence of highly positively charged amino acids. This allows the protein to be targeted to the highly negatively charged mitochondria.

As discussed above, in order to enter the mitochondria, a protein generally must interact with the mitochondrial import machinery, consisting of the Tim and Tom complexes (Translocase of the Inner/Outer Mitochondrial Membrane). With regard to the mitochondrial targeting signal, the positive charge draws the linked protein to the complexes and continues to draw the protein into the mitochondria. The Tim and Tom complexes allow the proteins to cross the membranes. Accordingly, one embodiment of the present disclosure delivers compositions of matter according to the present disclosure to the inner mitochondrial space utilizing a positively charged targeting signal and the mitochondrial import machinery. In another embodiment, PTD-linked compounds containing a mitochondrial localization signal do not seem to utilize the TOM/TIM complex for entry into the mitochondrial matrix.

In some embodiments, a STATS inhibiting moiety may be associated with a mitochondrial targeting sequence. A mitochondrial targeting sequence suitable for the present invention can be any peptide, or the like, that is capable of directing a therapeutic moiety to the mitochondrion of a cell. Still further, most of the mitochondrial targeting sequences (MTS) consist of 10 to 70 amino acids that are removed by 1 or 2 proteolytic steps once inside the mitochondria³⁰⁻³¹. Many mitochondrial proteins contain an intrinsic mitochondrial targeting sequence, typically, at the N-terminus Thus, a suitable mitochondrial targeting sequence may be derived from such intrinsic mitochondrial targeting sequence of mitochondrial proteins including, but not limited to, the FXN protein, cytochrome C oxidase IV (Cox IV) protein, ornithine transcarbylamse (OTC) protein, lipoamide dehygrogenase (LAD) protein, and malate dehydrogenase (MDH) protein. Suitable mitochondrial targeting sequences encompass both naturally-occurring sequences and modified sequences that retain mitochondrial targeting ability and can be produced using recombinant and synthetic methods or purified from natural sources.

Non-limiting examples for such moiety include but are not limited to Mitochondria targeting sequence (MTS) that comprises Met Ser Val Leu Thr Pro Leu Leu Leu Arg Gly Leu Thr Gly Ser Ala Arg Arg Leu Pro Val Pro Arg Ala Lys Ile His Ser Leu, as denoted by SEQ ID NO. 8. In yet another embodiment, the MTS may comprise H2N-Met-Leu-Ser-Leu-Arg-Gln-Ser-Ile-Arg-Phe-Phe-Lys-Pro-Ala-Thr-Arg-Thr-Leu-Cys-Ser-Ser-Arg-Tyr-Leu-Leu- as denoted by SEQ ID NO. 9.

Typically there are numerous additional sequences suitable, all of them characterized by 3-5 nonconsecutive Arg or Lys residues, often with Ser and Thr; no Glu or Asp residues.

It should be noted that in some embodiments, a suitable mitochondrial targeting sequence has a sequence at least 50%, 55%, 60%, 65%, 70%, 75%, 80%, 85%, 90%, 91%, 92%, 93%, 94%, 95%, 96%, 97%, 98%, 99% or more homologous to SEQ ID NOs:8 and 9.

Mitochondrial targeting peptides are typically attached to the STAT3 inhibiting moiety via either the C-terminus or the N-terminus of the peptide. The other end of the peptide may be typically unprotected or protected with a suitable protecting group. Suitable protecting groups are well known to those skilled in the art.

In certain embodiments, the STAT3 inhibitory moiety may be connected to the mitochondrial targeting moiety via a linker, optionally containing one or more branch points, to which multiple mitochondrial targeting agents are attached.

It should be appreciated that the composition of matter according to the invention may include or comprise different mitochondrial moieties. In some specific embodiments, where a peptide mitochondrial targeting moiety is used, said targeting moieties may include any one of MTS, MPPs and PTPs as defined above or any combination thereof. In the case of mitochondrial targeted STAT3 inhibitors containing a plurality of mitochondrial targeting moiety or agents, the agents may be the same or different. In some embodiments, a mitochondrial targeted STAT3 inhibitor is functionalized with multiple copies of the same agent. In alternative embodiments, a mitochondrial targeted STAT3 inhibitor is functionalized with two or more different mitochondrial targeting agents.

Mitochondrial targeting agents are known in the art, and include lipophilic cations that convey a positive charge to the compound under physiological conditions, such as cationic phosphonium and ammonium groups, peptide targeting moieties, and mitochondrial delivery vehicles.

Preferably the mitochondrial targeting agent does not permanently damage the mitochondrion, for example the mitochondrial membrane, or otherwise impair mitochondrial function.

It should be noted that the mitochondrial STAT3 inhibitors can be connected to one or more lipophilic cations that convey a positive charge to the compound under physiological conditions, such as cationic phosphonium and ammonium groups.

In the case of cationic phosphonium and ammonium groups, the selection of carbon substituents on the cationic atom will affect the target activity, the ability of the STAT3 inhibitory moiety to localize within the mitochondria, and the pharmacokinetic properties of the drug. Generally, the substituents on the cation are chosen to distribute the localization of the positive charge and to provide a lipophilic environment in the vicinity of the positive charge to shield the cation from direct interaction with lipophilic biological barriers. Additional pharmacokinetic properties, including oral bioavailability, volume of distribution, and clearance are also dependent on the balance between lipophilic and hydrophilic attributes.

Thus, in yet another embodiment, the mitochondrial targeting moiety used by the composition of matter according to the invention may be lipophilic cation. According to some embodiments, the lipophilic cation comprised within the composition of matter of the invention may be a phosphonium cation, an arsonium cation, an ammonium cation, flupritine, MKT-077, a pyridinium ceramide, a quinolium, a liposomal cation, a sorbitol guanidine, a cyclic guanidine or a rhodamine.

Mitochondrial targeting moieties are known in the art and include lipophilic cations as well as small molecules that convey a positive charge to the compound under physiological conditions. Representative mitochondrial targeting moieties include, but are not limited to alkyltriphenylphosphonium, tetraphenylphosphonium, tetraphenylarsonium, tribenzyl ammonium, phosphonium, polyarginine, polylysine, and delocalized lipophilic cations containing one to three carbimino, sulfimino, or phosphinimino units.

Liphophilic cations are used as effective mitochondrial targeting moieties as they can pass directly through phospholipid bilayers without requiring a specific uptake mechanism, and they accumulate substantially within mitochondria due to the large membrane potential. The large hydrophobic radius of the TPP cation enables it to pass easily through the phospholipid bilayer relative to other cations³². In one embodiment the disclosed compounds include TPP derivatives modified to increase hydrophobicity. For example, the hydrophobicity of the targeting moiety can be increased by increasing the length of the carbon chain linker.

It is believed that lipophilic cations are taken up from a positively charged cellular compartment into a negatively charged compartment until a sufficiently large concentration gradient is built up to equalize the electrochemical potential of the molecules in the two compartments.

As noted above, in certain embodiments, lipophilic cations are preferred mitochondrial targeting agents because they can pass directly through phospholipid bilayers without requiring a specific uptake mechanism, and they accumulate substantially within mitochondria due to the large membrane potential. The large hydrophobic radius of the triphenylphosphine (TPP) cation enables it to pass easily through the phospholipid bilayer relative to other cations. In some embodiments the disclosed compounds include at least one TPP derivatives modified to increase hydrophobicity. For example, the hydrophobicity of the targeting agent can be increased by increasing the length of the carbon chain linker.

Without wishing to be bound to one theory, it is believed that lipophilic cations are taken up from a positively charged cellular compartment into a negatively charged compartment until a sufficiently large concentration gradient is built up to equalize the electrochemical potential of the molecules in the two compartments. Thus, in further embodiments, the lipophilic cation comprised within the composition of matter according to the invention may be at least one Triphenylphosphonium (TPP).

In accordance with the present disclosure, the composition comprises at least one STAT3 inhibiting moiety. As used herein the term “STAT3 inhibiting moiety” denotes a moiety capable of restricting, limiting, hindering, preventing, decreasing, slowing down or arresting partially or completely the activity of STAT3 protein. The inhibition may be reversible inhibition or may be irreversible inhibition. It should be noted that such decrease in STAT3 activity may be evaluated as the decrease in activity of STAT3 as compared to the activity in the absence of the moiety. In certain embodiments, a decrease in the activity of STAT3 may be determined by one of the following: luminescence-based ATP determination assay, changes in mitochondrial membrane potential, oxygen consumption measurement and measurements of enzymatic respiratory chain complex activity. The terms “inhibition”, “decrease”, “moderation” or “attenuation” as referred to herein, relate to the retardation, restraining or reduction of a process, specifically the activation of mast cells, and/or release of mediator/s, by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%.

The inhibition of STAT3 in accordance with the present invention is not limited by a specific mechanism and may be due to direct targeting of the STAT3 protein for example by way of the SH2 domain inhibitors or dimerization inhibitors and the N-terminal domain inhibitors or by the indirect targeting of the upstream components of the STAT3 pathway. In certain embodiments, inhibitors directed to DNA binding domain of STAT3 affects its transcriptional activity and therefore may not be applicable for the purpose of the present invention. Without being bound by any theory, it was suggested by the inventors that the at least one STAT3 inhibiting moiety of the composition of matter of the invention, inhibits phosphorylation of Ser727 (serine at position 727) of STAT3. Further functional properties of the STAT3 inhibitory moiety are discussed in detail herein after.

Thus, according to some embodiments, the at least one STAT3 inhibiting moiety of the composition of matter of the invention, inhibits phosphorylation of Ser727 of STAT3. In yet some other embodiments, the STAT3 inhibiting moieties of the invention may inhibit, decrease or prevent the elevation in the phosphorylation of Ser727 of STAT3.

Thus, in certain embodiments, the composition of matter according to the invention may comprise at least one STAT3 inhibiting moiety that may be at least one of a small molecule based moiety, a nucleic acid based moiety, an amino acid based moiety and any combinations thereof.

It should be noted that the present disclosure is not limited to a specific STAT3 inhibiting moiety and is compatible with any known STAT3 inhibiting moiety. Non-limiting examples of inhibitory moiety include but are not limited to at least one small molecule moiety, at least one nucleic acid moiety, at least one amino acid moiety and any combinations thereof.

The term “moiety” is a functional description referring to the activity in inhibiting STAT3 or transport to the mitochondria. This term may refer to an independent single molecule, a complex or several molecules having this function, as well as to a region or domain inside a larger molecule. More specifically, “moiety” may refer to a molecule, a nucleic acid, an amino acid or a region in a molecule, a nucleic acid, an amino acid, or a complex of molecules, of nucleic acids, or amino acids.

In accordance with some embodiments, the at least one STAT3 inhibiting moiety may be an organic compound moiety such as a small molecule based moiety.

A small molecule in the context of the present disclosure refers to a low molecular weight organic compound, having a molecular weight lower than 900 Daltons. In accordance with the present disclosure, when referring to a small molecule it includes also crystalline and amorphous forms of those compounds, including, for example, polymorphs, pseudopolymorphs, solvates, hydrates, unsolvated polymorphs (including anhydrates), conformational polymorphs, and amorphous forms of the compounds, as well as mixtures thereof. “Crystalline form” or “polymorph,” as used herein include all crystalline and amorphous forms of a small molecule, including, for example, polymorphs, pseudopolymorphs, solvates, hydrates, unsolvated polymorphs (including anhydrates), conformational polymorphs, and amorphous forms, as well as mixtures thereof, unless a particular crystalline or amorphous form is referred to.

In accordance with the present disclosure, the term “small molecule” may include pharmaceutically acceptable forms of the recited compounds, including chelates, non-covalent complexes, prodrugs, and mixtures thereof. Further and in accordance with the preset disclosure, the term “small molecule” includes also pharmaceutically acceptable forms of a particular molecule and as such the term small molecule also encompasses pharmaceutically acceptable salts. The term “pharmaceutically acceptable salt(s)”, as used herein, refers to those salts of compounds (small molecule) describes herein that are safe and effective for pharmaceutical use in mammals and that possess the desired biological activity. Pharmaceutically acceptable salts include salts of acidic or basic groups present in compounds of the invention. It should be noted that the salts are not limited to salts with inorganic acids, such as hydrochlorate, phosphate, diphosphate, hydrobromate, sulfate, sulfinate, nitrate, and like salts; as well as salts with an organic acid, such as malate, maleate, fumarate, tartrate, succinate, citrate, acetate, lactate, methanesulfonate, p-toluenesulfonate, 2-hydroxyethylsulfonate, benzoate, salicylate, stearate, and alkanoate such as acetate, HOOC—(CH₂)ₙ—COOH where n is 0-4, and like salts.

Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. Certain compounds of the invention can form pharmaceutically acceptable salts with various amino acids. Suitable base salts include, but are not limited to, aluminum, calcium, lithium, magnesium, potassium, sodium, zinc, and diethanolamine salts.

When referring to small molecule it should be also noted that in case the molecule is obtained (present) as an acid addition salt, the free base can be obtained by basifying a solution of the acid salt. Alternatively, in case the compound is a free base, an addition salt, a pharmaceutically acceptable addition salt, may be obtained by dissolving the free base in a suitable organic solvent and treating the solution with an acid, in accordance with conventional procedures for preparing acid addition salts from base compounds.

It should be appreciated that the compounds (small molecule) described herein may encompass also solvates thereof. As used herein, the term “solvate” refers to a complex of variable stoichiometry formed by an interaction of a compound (solute) and a solvent. Such solvents for the purpose of the invention may not interfere with the biological activity of the solute. Examples of suitable solvents include pharmaceutically acceptable solvates, such as hydrates, including monohydrates and hemi-hydrates, for example but are not limited to, water, methanol and ethanol.

The present disclosure also relates to prodrug derivatives of the compounds of the invention. The term “prodrug” refers to pharmacologically precursors of a drug that may be converted into a therapeutically active compounds of the invention under physiological conditions in vivo or in vitro, for example, when they undergo solvolysis, or enzymatic degradation in blood, or in cells. In some embodiments, the “prodrugs” described herein include any compound that becomes a compound described herein when administered to a patient, e.g., upon metabolic processing of the prodrug. Examples of prodrugs include derivatives of functional groups, such as a carboxylic acid group such as but are not limited to, carboxylic acid esters such as alkyl esters, hydroxyalkyl esters, arylalkyl esters, and aryloxyalkyl esters.

As such, in accordance with the present disclosure, small molecule compounds encompass at least one of enantiomers thereof, racemic mixtures thereof, salts thereof (being pharmaceutically acceptable or unacceptable), internal salts thereof, hydrates thereof, polymorphs thereof, prodrugs thereof and mixtures of any one form thereof. Any of these enantiomers, racemic mixtures, salts (being pharmaceutically acceptable or unacceptable), internal salts, hydrates, polymorphs, prodrugs and mixtures apply to any one of the small molecule described herein.

The present invention thus also contemplates derivatives of any of the inhibitors of the invention. The term “derivative” as used herein refers to a chemically modified small molecule compound derived from a parent compound of the invention that differs from the parent compound by one or more elements (such as atoms, bonds etc), substituents, chemical groups and/or functional groups. The derivative may have the same or similar biological properties/activities as the parent compound, as defined herein. For those versed in the art, a derivative thereof can be obtained during compound optimization and may involve at least one modification as detailed above of any of the parent compound/s that will be described in more detail herein after.

In some embodiments, the at least one STAT3 inhibiting moiety may be at least one small molecule capable of inhibiting STAT activity. In some further embodiments, the at least one small molecule may be any one of STAT3 Inhibitor V, 6-Nitrobenzo[b]thiophene 1,1-dioxide (Stattic), (1E,6E)-1,7-Bis(4-hydroxy-3-methoxyphenyl)-1,6-heptadiene-3,5-dione (curcumin), N-(1′,2-Dihydroxy-1,2′-binaphthalen-4′-yl)-4-methoxybenzenesulfonamide (C188-9), N-Hexyl-2-(1-naphthalenyl)-5-[[4-(phosphonooxy)phenyl]methyl]-4-oxazolecarboxamide (S3I-M2001), 8-hydroxy-3-methyl-3,4-dihydrotetraphene-1,7,12(2H)-trione (STA-21), 2-Hydroxy-4-[[2-[[(4-methylphenyl)sulfonyl]oxy]acetyl]amino]benzoic acid (S3I-201), Cepharanthine; Cucurbitacin I; Cucumis sativus L; Niclosamide; Cryptotanshinone; SD 1008; Stat3 Inhibitor III, WP1066; Nifuroxazide; Stat3 Inhibitor VI, S3I-201; STA-21; Kahweol; STAT3 Inhibitor IX, Cpd188; STAT3 Inhibitor VI, S3I-201; STAT3 Inhibitor VII Ethyl-1-(4-cyano-2,3,5,6-tetrafluorophenyl)-6,7,8-trifluoro-4-oxo-1,4-dihydroquinoline-3-carboxylate; STAT3 Inhibitor VIII, 5,15-DPP, STAT3 Inhibitor X, HJB; STAT3 Inhibitor XII, SPI; STAT3 Inhibitor XI, STX-0119; STAT3 Inhibitor XIV, LLL12; FLLL32; FLLL62 and the like. The present disclosure also encompasses any derivatives, analogs or any combinations thereof or any vehicle, matrix, nano- or micro-particle comprising the same.

In some embodiments, the at least one STAT3 inhibiting moiety is STAT3 Inhibitor V, 6-Nitrobenzo[b]thiophene 1, 1-dioxide (Stattic) or any derivative thereof. More specifically, Stattic™ (by Enzo) is a small molecule STAT3-specific inhibitor which blocks activation, dimerization and nuclear translocation via binding to the STAT3 SH2 domain.

In some embodiments, the composition of matter comprises at least one STAT3 inhibiting moiety associated with at least one TPP. In some further embodiments, the composition of matter comprises a small molecule or a derivative thereof associated with TPP.

In some specific embodiments, the composition of matter comprises STAT3 Inhibitor V, 6-Nitrobenzo[b]thiophene 1,1-dioxide (Stattic) associated with TPP. It should be noted that said mitochondrial-targeted STAT3 inhibitor is referred to herein as MitoS.

Association between 6-Nitrobenzo[b]thiophene 1,1-dioxide and TPP may be done using any suitable synthetic method known in the art.

In some other embodiments, the at least one STAT3 inhibiting moiety may be a member of curcuminoid. A curcuminoid as used herein refer to a linear diarylheptanoid, with molecules such as curcumin or derivatives thereof. In some embodiments, the at least one STAT3 inhibiting moiety is curcumin or diferuloylmethane (C₂₁H₂₀O₆), also denoted by the IUPAC name as (1E,6E)-1,7-Bis(4-hydroxy-3-methoxyphenyl)-1,6-heptadiene-3,5-dione. In some further embodiments, the at least one STAT3 inhibiting moiety may be demethoxycurcumin or 4-hydroxycinnamoyl(feroyl) methane (C₂₀H₁₈O₅) or curcumin II, BHCFM, also denoted by the IUPAC name (1E,6E)-1,6-Heptadiene-3,5-dione, 1-(4-hydroxy-3-methoxyphenyl)-7-(4-hydroxyphenyl). In some further embodiments, the at least one STAT3 inhibiting moiety may be bisdemethoxycurcumin [bis-ane] (C₁₉H₁₆O₄) or Bis(4-hydroxycinnamoyl)methane, BHCMT or curcumin III also denoted by the IUPAC name as (1E,6E)-1,7-bis(4-hydroxyphenyl) hepta-1,6-diene-3,5-dione. When referring to curcumin, it should be noted as having two aryl rings containing ortho-methoxy phenolic OH⁻ groups that are symmetrically linked to a β-diketone moiety. The occurrence of intramolecular hydrogen atoms transfer at the β-diketone chain of curcumin leads to the existence of keto and enol tautomeric conformations in equilibrium that can exist in several cis and trans forms. The amount of keto-enol-enolate of the heptadienone moiety in equilibrium may affect the physicochemical properties and anti-oxidant activities of curcumin. In acidic and neutral conditions (i.e. pH 3-7), the major constituents present are curcumin molecules in bis-keto form where curcumin acts as a potent proton donor. This is attributable to the presence of a highly activated carbon atom in the heptadienone linkage between the two-methoxy phenol rings of bis-keto form of curcumin. However, in situations (i.e. pH>8) where the enolate form of the heptadienone chains predominates, curcumin acts instead as an electron donor. The presence of enolate in solution is found to be important in the radical-scavenging ability of curcumin. Curcumin has three ionisable protons contributed by the enolic proton (approximate pKa of 8.5) and two phenolic OH⁻ groups (pKa of 10-10.5).

In some other embodiments, the composition of matter comprises curcumin or a derivative thereof associated with at least two TPP.

As shown in Examples 10-12, the inventors used curcumin-based mitochondrial-targeted STAT3 inhibitors. Thus, in some embodiments, the composition of matter comprises (1E,6E)-1,7-Bis(4-hydroxy-3-methoxyphenyl)-1,6-heptadiene-3,5-dione or any derivative/s, salt/s or ester/s thereof associated with TPP. The association between (1E,6E)-1,7-Bis(4-hydroxy-3-methoxyphenyl)-1,6-heptadiene-3,5-dione may be with two TPP moieties. It should be noted that said compound is referred to herein and in the following Examples as mito-cur-1 (or Mitocur-1).

In some other embodiments, the composition of matter comprises (1E,6E)-1,7-bis(4-hydroxyphenyl)hepta-1,6-diene-3,5-dione associated with TPP. The association between (1E,6E)-1,7-bis(4-hydroxyphenyl)hepta-1,6-diene-3,5-dione may be with two TPP moieties. It should be noted that said compound is referred to herein and in the following Examples as mito-cur-3 (or Mitocur-3).

In certain embodiments, the STAT3 inhibiting moiety may be encapsulated in a nano- or micro-particle, any matrix or any carrier.

More specifically, in accordance with some embodiments, the at least one STAT3 inhibitory moiety may be held within a carrier. The carrier in accordance with the present disclosure may be such that is capable of holding in an intra cavity the at least one STAT3 inhibitory moiety.

In some embodiments, the carrier in accordance with the present disclosure is characterized as comprising at least one mitochondrial targeting moiety on the surface.

The carrier may be any one of nanoparticle, a microparticle, or an organized collection of lipids. The term “nanoparticle” is used herein to denote any microscopic particle smaller than about 100 nm in diameter. In some other embodiments, the carrier is an organized collection of lipids.

In accordance with the present disclosure, the carrier comprising the at least one mitochondrial targeting moiety may be considered as a mitochondrial delivery vehicle, such as a lipid raft, mitochondrially targeted nanoparticle, or mitochondriotropic liposome.

The at least one mitochondrial targeting moiety is present in at least 10%, at least 20%, at least 30%, at least 40%, at least 50%, at least 60%, at least 70%, at least 80%, at least 90% and at least 99% or more, of the surface of the carrier.

In some embodiments, the at least one mitochondrial targeting moiety is connected (conjugated) to the carrier surface via chemical or physical bonding as described herein below. The association of the at least one mitochondrial targeting moiety with the carrier may be direct or may be via a linker. Non-limiting examples for conjugation of TPP with the liposome are disclosed for example in WO 2996094203, that is incorporated herein by reference in its entirety According to some embodiments, the at least one mitochondrial targeting moiety may be conjugated to a polymer. In one embodiment, the polymer is polyethylene glycol (PEG).

The targeted carrier may comprise organized collection of lipids that may form a lipid vesicle. The lipids are referred to herein collectively by the term “structure forming lipids”. As such, a structure forming lipid denotes a lipid that can form a vesicle, e.g. a liposome.

The structure forming lipids may be defined by their phase transition temperature. As used herein the term “phase transition temperature” refers to a temperature required to induce a change in the lipid physical state from the ordered gel phase (solid), where the hydrocarbon chains are fully extended and closely packed, to the disordered liquid crystalline phase, where the hydrocarbon chains are randomly oriented and fluid.

In some embodiments, the structure forming lipids are defined by those having a phase transition temperature from solid phase to liquid phase between about 20° C. to 60° C., at times, below 60° C., or below 50° C. or below 40° C. When more than one structure forming lipid is used, the total lipids have together a phase transition temperature from solid phase to liquid phase within the recited range or below the recited upper limit There are various lipids known in the art to have a phase transition temperature below 60° C.

In some embodiments, the at least one STAT3 inhibitory moiety is held by a plurality of lipid organized structures, referred to herein as lipid particles. The lipid nanoparticles (either nano or micro sized) may include, without being limited thereto, vesicles, micelles, liposomes and the like. In some embodiments, the at least one STAT3 inhibitory moiety is encapsulated within the intraliposomal core of liposomes. In some other embodiments, the at least one STAT3 inhibitory moiety is carried by lipid micelles.

The at least one STAT3 inhibitory moiety may be loaded into the liposomes by any technique known in the art for loading active agent into liposomes, these include, inter alia, active loading, passive loading.

When referring to the structure forming lipids it is to be understood to mean any biocompatible lipid that can assemble into an organized collection of lipids (organized structure). In some embodiments, the lipid may be natural, semi-synthetic or fully synthetic lipid, as well as electrically neutral, negatively or positively charged lipid.

In some embodiments, the lipid is a naturally occurring phospholipid. Examples of lipids forming glycerophospholipids include, without being limited thereto, glycerophospholipid, phosphatidylglycerols (PG) including dimyristoyl phosphatidylglycerol (DMPG); phosphatidylcholine (PC), including egg yolk phosphatidylcholine, dimyristoyl phosphatidylcholine (DMPC), 1-palmitoyl-2-oleoylphosphatidyl choline (POPC), hydrogenated soy phosphatidylcholine (HSPC), distearoylphosphatidylcholine (DSPC); phosphatidic acid (PA), phosphatidylinositol (PI), phosphatidylserine (PS).

Examples of cationic lipids may include, for example, 1,2-dimyristoyl-3-trimethylammonium propane (DMTAP) 1,2-dioleyloxy-3-(trimethylamino) propane (DOTAP); N-[1-(2,3,-ditetradecyloxy)propyl]-N,N-dimethyl-N-hydroxyethylammonium bromide (DMRIE); N-[1-(2,3,-dioleyloxy)propyl]-N,N-dimethyl-N-hydroxy ethyl-ammonium bromide (DORIE); N-[1-(2,3-dioleyloxy)propyl]-N,N,N-trimethylammonium chloride (DOTMA); 3β[N—(N′,N′-dimethylaminoethane)carbamoly] cholesterol (DC-Chol); and dimethyl-dioctadecylammonium (DDAB), N-[2-[[2,5-bis[3-aminopropyl)amino]-1-oxopentyl]amino]ethyl]-N,N-dimethyl-2,3-bis[(1-oxo-9-octadecenyl)oxy]-1-propanaminium (DOSPA), and ceramide carbamoyl spermine (CCS), or the neutral lipid dioleoylphosphatidyl ethanolamine (DOPE) derivatized with polylysine to form a cationic lipopolymer.

The lipids may be combined with other lipid compatible substances, such as, sterols, lipopolymers etc. A lipopolymer is a lipid modified by inclusion in its polar headgroup a hydrophilic polymer. The polymer headgroup of a lipopolymer is preferably water-soluble. In some embodiments, the hydrophilic polymer has a molecular weight equal or above 750 Da. There are numerous polymers which may be attached to lipids to form such lipopolymers, such as, without being limited thereto, polyethylene glycol (PEG), polysialic acid, polylactic (also termed polylactide), polyglycolic acid (also termed polyglycolide), apolylactic-polyglycolic acid, polyvinyl alcohol, polyvinylpyrrolidone, polymethoxazoline, polyethyloxazoline, polyhydroxyethyloxazoline, polyhydroxypropyloxazoline, polyaspartamide, polyhydroxypropyl methacrylamide, polymethacrylamide, polydimethylacrylamide, polyvinylmethylether, polyhydroxyethyl acrylate, derivatized celluloses such as hydroxymethylcellulose or hydroxyethylcellulose. The polymers may be employed as homopolymers or as block or random copolymers. The lipids derivatized into lipopolymers may be neutral, negatively charged, as well as positively charged. The most commonly used and commercially available lipids derivatized into lipopolymers are those based on phosphatidyl ethanolamine (PE), usually, distearoylphosphatidylethanolamine (DSPE).

In some embodiments, the structure forming lipids are combined with other lipids, such as a sterol. Sterols and in particular cholesterol are known to have an effect on the properties of the lipids organized structure (lipid assembly), and may be used for stabilization, for affecting surface charge, membrane fluidity.

In some embodiments, a sterol, e.g. cholesterol is employed in order to control fluidity of the lipid structure. The greater the ratio sterol:lipids (the structure forming lipids), the more rigid the lipid structure is.

Liposomes are often distinguished according to their number of lamellae and size. The liposomes employed in the context of the present disclosure may be multilamellar vesicles (MLVs), multivesicular vesicles (MVVs), small unilamellar vesicles (SUVs), large unilamellar vesicles (LUVs) or large multivesicular vesicles (LMVV). In some specific and non-limiting embodiments, the liposomes are as described in WO 2996094203 and US 2008/0095834 that are incorporated herein by reference in their entireties.

The at least one STAT3 inhibitory moiety is associated with the carrier. The term “association” may be used interchangeably with the term “entrapped”, “attachment”, “linked”, “embedded”, “absorbed” and the like, and contemplates any manner by which the at least one STAT3 inhibitory moiety is held. This may include for example, physical or chemical attachment to the carrier. Chemical attachment may be via a linker, such as polyethylene glycol. The association provides capturing of the at least one STAT3 inhibitory moiety by the carrier such that the release of the at least one STAT3 inhibitory moiety may be controllable.

In more specific embodiments, the mitochondrial targeting agent can also be a mitochondrial delivery vehicle, such as a lipid raft, mitochondrial-targeted nanoparticle, or mitochondriotropic liposome. In such cases, one or more mitochondrial-targeted STAT3 inhibitors can be associated with, encapsulated within, dispersed in or on, or covalently attached to the mitochondrial delivery vehicle.

In preferred embodiments, the STAT3 inhibitors are encapsulated, non-covalently associated with, or dispersed in the mitochondrial delivery vehicle. In these embodiments, a suitable STAT3 inhibitor can be incorporated without any covalent modification. Representative inhibitors include, but are not limited to any one of STAT3 Inhibitor V, 6-Nitrobenzo[b]thiophene 1,1-dioxide (Stattic); (1E,6E)-1,7-Bis(4-hydroxy-3-methoxyphenyl)-1,6-heptadiene-3,5-dione (curcumin); STAT3 Inhibitor XIII, C188-9.

In certain embodiments, STAT3 inhibitors may be encapsulated, coupled to, or otherwise associated with mitochondriotropic liposomes. Mitochondriotrophic liposomes are cationic liposomes that can be used to deliver an encapsulated agent to the mitochondria of a cell. Mitochondriotropic liposomes are known in the art. See, for example, U.S. Patent Application Publication No. US 2008/0095834 to Weissig, et al, which is incorporated herein by reference in its entirety. Mitochondriotropic liposomes are liposomes which contain a hydrophobized amphiphilic delocalized cation, such as a triphenylphosphonium or a quinolinium moiety, incorporated into or conjugate to the lipid membrane of the liposome. As a result, the liposomes can be used to deliver compounds incorporated within them to the mitochondria.

In other embodiments, STAT3 inhibitors may be encapsulated within, dispersed in, associated with, or conjugated to a nanoparticle functionalized with one or more mitochondrial targeting agents. For example, the nanoparticle may contain one or be functionalized with one or more lipophilic cations or polypeptide targeting agents.

The nanoparticles may be formed from one or more polymers, copolymers, or polymer blends. In some embodiments, the one or more polymers, copolymers, or polymer blends are biodegradable. Examples of suitable polymers include, but are not limited to, polyhydroxyacids such as poly(lactic acid), poly(glycolic acid), and poly(lactic acid-co-glycolic acids); polycaprolactones; poly(orthoesters); polyanhydrides; poly(phosphazenes); poly(hydroxyalkanoates); poly(lactide-co-caprolactones); polycarbonates such as tyrosine polycarbonates; polyamides (including synthetic and natural polyamides), polypeptides, and poly(amino acids); polyesteramides; polyesters; poly(dioxanones); poly(alkylene alkylates); hydrophobic polyethers; polyurethanes; polyetheresters; polyacetals; polycyanoacrylates; polyacrylates; polymethylmethacrylates; polysiloxanes; poly(oxyethylene)/poly(oxypropylene) copolymers; polyketals; polyphosphates; polyhydroxyvalerates; polyalkylene oxalates; polyalkylene succinates; poly(maleic acids), poly(alkylene glycols) such as polyethylene glycol (PEG), poly(propylene glycol) (PPG), and copolymers of ethylene glycol and propylene glycol, poly(oxyethylated polyol), poly(olefmic alcohol), polyvinylpyrrolidone), poly(hydroxy alkylmethacrylamide), poly(hydroxyalkylmethacrylate), poly(saccharides), poly(vinyl alcohol), as well as blends and copolymers thereof. Techniques for preparing suitable polymeric nanoparticles are known in the art, and include solvent evaporation, hot melt particle formation, solvent removal, spray drying, phase inversion, coacervation, and low temperature casting. In some cases, the mitochondrial targeting agents are polypeptides that are covalently linked to the surface of the nanoparticle after particle formulation. In other cases, the mitochondrial targeting agents are lipophilic cations that are covalently bound to the particle surface. In some cases, a cationic polymer is incorporated into the particle to target the particle to the mitochondria.

STAT3 inhibitors can also be targeted to the mitochondria using lipid rafts or other synthetic vesicle compositions. The lipid raft compositions can include cholesterol, and one or more lipids selected from the group consisting of sphingomylein, gangliosides, phosphatidylethanolamine, phosphatidylserine, phosphatidylcholine, phosphatidylinositol, and a mitochondrial targeting agent. In certain embodiments, a polypeptide targeting agent may be inserted into the lipid raft to target the raft to the mitochondria. The lipid rafts can be prepared and loaded with one or more STAT3 inhibitors using methods known in the art.

Mitochondrialy targeted STAT3 inhibitors may optionally contain a linker which connects the STAT3 inhibitor to one or more mitochondrial targeting agents. The linker can be inert, or the linker can have biological activity. The linker must be at minimum bivalent; however, in some embodiments, the linker can be bound to more than one active agent, in which case, the linker is polyvalent.

The linker can be composed of any assembly of atoms, including oligomeric and polymeric chains, which functions to connect one or more of the mitochondrial targeting agents described above to a STAT3 inhibitor. In some cases, the linker may be an oligomeric and polymeric chain, such as an oligo- or polyethylene glycol chain, or an oligo- or poly(amino acid) chain. Peptide linkers include peptides that can be cleaved once the compound enters the mitochondria. For example, in some cases, the peptide linker is a mitochondrial localization signal as discussed in detail above. In other cases the linker is a non-polymeric organic functional group, such as an alkyl group or an alkylaryl group. In certain embodiments, the linker may be hydrophilic to facilitate passage of the creatine compound across biological membranes.

In many cases, the linker is a linear chain; however, in some embodiments, the linker/s may contain one or more branch points. In the case of branched linker, the terminus of each branch point can be functionalized with a mitochondrial targeting agent. In one such embodiment, a dendritic linker is used, with the STAT3 inhibitor being bound to the focal point of the dendrimer, and multiple mitochondrial targeting agents being bound to the ends of the dendritic branches.

In some embodiments, the linker includes one or more cleavable subunits, such as a disulfide group, a hydrazone group, or a peptide group which can be cleaved by proteolytic enzymes within a cell. In alternative embodiments, the linker contains one or more hydrolysable subunits, such as an ester group. The linker can also contain one or more covalent or non-covalent functional groups to facilitate the assembly and/or separation of the STAT3 inhibitor from the attached mitochondrial targeting agent, including, but not limited to one or more metal complexes, such as polyhistidine-nickel chelate complexes, one or more heteroaromatic rings (such as triazole rings formed by the cycloaddition of an alkyne and an azide), one or more hydrogen bond donor-acceptor pairs, one or more biomolecule/bioconjugate pairs (such as biotin-avidin or biotin-streptavidin pair), as well as combinations thereof.

One or more ends of the linking group may include a functional group used to facilitate attachment of a STAT3 inhibitor and a mitochondrial targeting agent. The functional group may be an atom or group of atoms that contains at least one atom that is neither carbon nor hydrogen. In some embodiments, the functional group may be a halo functional group, such as a fluoro, chloro, bromo, or iodo group; an oxygen-containing functional group, such as a hydroxyl, carbonyl, aldehyde, acetal, hemiacetal, hemiketal, ester, orthoester, carboxylic acid, carboxylate, or ether group; a nitrogen-containing functional group, such as an amide, amine, imine, azide, cyanate, nitrate, nitrile, nitrite, or pyridyl group; phosphorus containing functional groups, such as a phosphate or phosphono group; or a sulfur-containing functional group, such as a sulfide, sulfonyl, sulfonamido, sulfino, sulfo, sulfmyl, sulfhydryl, carbonothioyl, or disulfide group.

In some embodiments, the functional group is a carboxylic acid, a chemical moiety which can be derived from a carboxylic acid by one or more chemical reactions (such as a condensation reaction to form an ester, amide, or thioester, or a reduction reaction to form an aldehyde or alcohol), an analog of a carboxylic acid in which one or more of the atoms is replaced by a sulfur atom, or an analog of a chemical moiety which could be derived from a carboxylic acid by one or more chemical reactions in which one or more of the atoms is replaced by a sulfur atom. In certain embodiments, the functional group is a secondary amide, tertiary amide, secondary carbamate, tertiary carbamate, urea, carbinol, ether, carboxylic acid, or ester.

STAT3 inhibitors functionalized with one or more mitochondrial targeting agents can be synthesized by reacting a suitable STAT3 inhibitor with either a mitochondrial targeting agent or with a linking group. In some embodiments, the STAT3 inhibitor and the mitochondrial targeting agent are covalently connected by linker. In such instances, the mitochondrial targeting agent may be prepared by first coupling the mitochondrial targeting agent to the linking group, then reacting the linking group with the STAT3 inhibitor. Alternatively, these compounds can be prepared by first reacting the linking group with the STAT3 inhibitor, and then coupling the linking group to the mitochondrial targeting agent.

The appropriate route for synthesis of a given mitochondrial targeted STAT3 inhibitor can be selected in view of the linking group desired, the mitochondrial targeting agent desired, the structure of the STAT3 inhibitor, as well as the structure of the compound as a whole as it relates to compatibility of functional groups, protecting group strategies, and the presence of labile bonds. Mitochondrialy targeted STAT3 inhibitors may be prepared by covalent modification of commercially available STAT3 inhibitors, including, any one of STAT3 Inhibitor V, 6-Nitrobenzo[b]thiophene 1,1-dioxide (Stattic); (1E,6E)-1,7-Bis(4-hydroxy-3-methoxyphenyl)-1,6-heptadiene-3,5-dione (curcumin); STAT3 Inhibitor XIII, C188-9, or any known STAT3 inhibitor.

According with some other embodiments, the composition of matter comprises liposomes encapsulating in their intraliposomal core at least one 6-Nitrobenzo[b]thiophene 1,1-dioxide (static) and having at least one TPP associated onto the outer liposomal surface. In certain embodiments, said mitochondrial-targeted STAT3 inhibitor is referred to herein as LipoS or Lipo-stattic.

In yet another embodiment, the STAT3 inhibiting moiety may comprise an amino acid based STAT3 inhibitor. Non limiting examples include peptides, polypeptides and antibodies. “Amino acid/s” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. “Polypeptide”, “peptide” and “protein” are used interchangeably herein to refer to a polymer of amino acid residues. The terms apply to naturally occurring amino acid polymers, as well as, amino acid polymers in which one or more amino acid residue is an artificial chemical mimetic of a corresponding naturally occurring amino acid.

More specifically, a STAT3 inhibitor used for the composition of matter according to the invention may be any one of STAT3 Inhibitor Peptide Ac-PpYLKTK-OH, as denoted by SEQ ID NO. 10, STAT3 Inhibitor I; STAT3 Inhibitor Peptide, Cell-Permeable PYLKTK-mts, having the sequence NH2-PYLKTKAAVLLPVLLAAP-CO2H as denoted by SEQ ID NO. 11; STAT3 Inhibitor Peptide II, ErbB2-Selective, Cell-Permeable H-YGRKKRRQR-G-FCDGFYACYKDV-PpYL-OH, Cyclic, P3-AHNP-STAT3BP, as denoted by SEQ ID NO. 13. Protein inhibitor of activated STAT family; Suppressor of cytokine signaling (SOCS) family; Rac1 small GTPase; Grim19 and P53.

As shown in Example 6, the inventors used PIAS3 targeted to the mitochondria to inhibit activation of MC. Thus, in some specific embodiments the STAT3 inhibiting moiety used in the composition of matter according to the invention may be at least one molecule of PIAS3 or any fragments, analogs and derivatives thereof. PIAS (protein inhibitor of activated STAT (signal transducer and activator of transcription) family of transcriptional modulators. The protein functions as a SUMO (small ubiquitin-like modifier)-E3 ligase which catalyzes the covalent attachment of a SUMO protein to specific target substrates. PIAS3 indirectly binds to several transcription factors and either blocks or enhances their activity.

It should be noted that in certain embodiments, PIAS3 as used herein may refer to the human PIAS3 as disclosed in AAH30556.2. In more specific embodiments said PIAS3 comprises the amino acid sequence as denoted by SEQ ID NO. 14. In further embodiments, said PIAS3 is encoded by the nucleic acid sequence as denoted by GenBank Accession number BC030556.1. It should be appreciated that the invention further encompasses the mouse PIAS3, specifically, Mus musculus PIAS3 as disclosed in AAH23128.1. In more specific embodiments said Mus musculus PIAS3 comprises the amino acid sequence as denoted by SEQ ID NO. 15. In further embodiments, said Mus musculus PIAS3 is encoded by the nucleic acid sequence as denoted by GenBank Accession number AK077551.1.

In more specific embodiments, at least one STAT3 inhibiting moiety of the composition of matter according to the invention may be at least one PIAS3 or any fragments or peptides thereof. It should be noted that in certain embodiments, the PIAS3 may be attached to or associated with at least one mitochondrial targeting peptide. In more specific embodiments, at least one PIAS3 molecule or any fragments and peptides thereof may be attached to at least one MTS that comprises Met Ser Val Leu Thr Pro Leu Leu Leu Arg Gly Leu Thr Gly Ser Ala Arg Arg Leu Pro Val Pro Arg Ala Lys Ile His Ser Leu, as denoted by SEQ ID NO. 8.

In more specific embodiments, the composition of matter according to the invention may comprise the mitochondrial-targeted PIAS3 referred to herein as PIAS3-mito or PIAS3-MTS. In some specific embodiments, said PIAS3-mito may comprise the amino acid sequence as denoted by SEQ ID NO. 16 or any homologs, variants or derivatives thereof. In yet some embodiments, the invention relates to a variant having a myc-tag, as denoted by SEQ ID NO. 18.

It should be noted that the invention provides the PIAS3-MTS (or PIAS3-mito) molecule of the invention, specifically or SEQ ID NO. 16 or 18, but also encompasses any homologs, variants, fragments and derivatives thereof. A “variant” of such molecule is meant to refer a naturally occurring molecule substantially similar to either the entire molecule or a fragment thereof. The term “derivatives” as used herein mean a molecule comprising the amino acid sequence of the PIAS3-mito molecule, as denoted by any one of SEQ ID NO. 16 or 18, with any insertions, deletions, substitutions and modifications that do not interfere with the ability of said molecule to inhibit the mitochondrial activity of STATS and thereby inhibit mast cell activation. A derivative should maintain a minimal homology to said amino acid sequence, e.g. even less than 30%, specifically, about 60% identity, preferably 65%, 70%, 75%, 80%, 85%, 90%, 91%, 92%, 93%, 94%, 95%, 96%, 97%, 98%, 99%, or higher identity over a specified region or over the entire molecule. It should be appreciated that the terms “insertions” and “deletions” are meant any addition or reduction, respectively, of amino acid residues to the PIAS3-mito used by the invention, between 1 to 50 amino acid residues, preferably, between 1 to 20 amino acid residues and most preferably, between 1 to 10 amino acid residues. More particularly, insertions or deletions, respectively, may include addition or reduction of 1, 2, 3, 4, 5, 6, 7, 8, 9 or 10 amino acid residues to the PIAS3-mito molecule of SEQ ID NO. 16 or 18.

In yet further embodiments, it should be noted that the invention also encompasses the nucleic acid sequence encoding said PIAS3-mito, as well as any expression vector comprising the same and any host cell transfected by said vector. In non limiting examples the nucleic acid sequence encoding the PIAS3-mito of the invention may comprise the sequence as denoted by SEQ ID NO. 17. In yet some embodiments, the invention relates to a variant having a sequence encoding myc-tag, as denoted by SEQ ID NO. 19. It should be noted that the invention further encompasses any derivatives, variants and homologs of the nucleic acid sequences disclosed herein.

It should be appreciated that the invention provides the PIAS3-MTS inhibitor of the invention in an isolated or purified form. The term “isolated”, “purified” or “substantially purified”, when applied to a nucleic acid or protein, such as the PIAS3-MTS molecules, denotes that the nucleic acid or protein is essentially free of other cellular components with which it is associated in the natural state. It is preferably in a homogeneous state, although it can be in either a dry or aqueous solution. Purity and homogeneity are typically determined using analytical chemistry techniques such as polyacrylamide gel electrophoresis or high performance liquid chromatography. A protein which is the predominant species present in a preparation is substantially purified.

In further embodiments, the STAT3 inhibitor may be nucleic acid based inhibiting moiety. The terms “Nucleic acid/s” and “polynucleotide” are used interchangeably herein to refer to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring. Examples of such analogs include, without limitation, phosphorothioates, phosphoramidates, methyl phosphonates, chiral-methyl phosphonates, 2-O-methyl ribonucleotides, peptide-nucleic acids (PNAs). As appreciate by one of skill in the art, the complement of a nucleic acid sequence can readily be determined from the sequence of the other strand. Thus, any particular nucleic acid sequence set forth herein also discloses the complementary strand.

Non limiting examples for nucleic acid based inhibiting moiety include siRNA, anti-sense, miRNA and the like. Examples for such inhibitors include miR-130b; MicroRNA-124; microRNA-1234; MiRNA-20; MiRNA-106a; miR-17 cluster family members (miR-17-5p, miR-17-3p, miR-18a, miR-19a miR-20a, miR-19b and miR92-1); miR-20a; miR-337-3p; miR-125b; miR-106b-25; miR-106a-363: miR-20b.

It must be appreciated that the composition of matter according to the invention, as well as the compositions and methods described herein after, may comprise any of the STAT3 inhibiting moieties described herein or any combination thereof and may be linked or associated to at least one of any of the mitochondrial targeting moieties discussed herein or to any combination thereof.

As shown by the following examples, the inventors clearly show that mitochondrial-targeted STAT3 inhibiting moiety inhibits MC activation.

Thus, a further aspect of the invention relates to a pharmaceutical composition comprising a pharmaceutically acceptable carrier and, as an active ingredient, at least one STAT3 inhibiting moiety or any vehicle, matrix, nano- or micro-particle comprising the same, associated with at least one mitochondrial targeting moiety, or any composition of matter comprising the same as described by the invention. In more specific embodiments, the active ingredient may be present in an amount effective for inhibiting and/or decreasing mast cell (MC) degranulation, while retaining STATS nuclear function's.

Mast cells (referred to herein as MS), also known as a mastocytes or a labrocytes are derived from the myeloid stem cells and contain many granules rich in histamine and heparin, very similar to basophil granulocytes. Mast cells comprise a normal component of the connective tissue that plays an important role in immediate (type I) hypersensitivity and inflammatory reactions by secreting a large variety of chemical mediators from storage sites in their granules upon stimulation. Mast cells, and their circulating counterparts the basophiles, possess surface receptors known as FcεRI that are specific for IgE heavy chains.

The event that initiates immediate hypersensitivity is the binding of antigen to the antigen-binding site in IgE on the mast cell or basophil surface. Both cell types are activated by cross-linking of FcεRI molecules, which is thought to occur by binding multivalent antigens to the attached IgE molecules.

Therefore, mast cells activation or degranulation, as used herein, involve release of preformed granules and the secretion of eicosanoids, cytokines and chemokines. Mast cells may also be activated by mechanisms other than cross linking FcεRI, such as in response to mononuclear phagocyte-derived chemocytokines, to T cell-derived cytokines and to complement-derived anaphylatoxins.

In certain embodiments, the present invention relates to mast cell activation caused by cross-linking of FcεRI. However, it should be appreciated that the invention may be also applicable in inhibiting MC activation or degranulation caused by any of the above.

More specifically, when antigen binds to IgE molecules attached to the surface of mast cells, a variety of mediators are released which give rise to increased vascular permeation, vasodilatation, bronchial, visceral smooth muscle contraction, and local inflammation.

Mediators released from mast cells may be divided into two broad classes, pre-formed or secretory granule associated mediators and nonpreformed or newly synthesized mediators. The pre-formed mediators include biogenic amines, most notably histamine. The pre-formed mediators also comprise granule macromolecules such as proteoglycans, most notably heparin and chondroitin sulfate E; chemotactic factors such as eosinophil and neutrophil chemotactic factors of anaphylaxis; and enzymes such as proteases, tryptase, chymase, cathepsin G-like enzyme, elastase, carboxypeptidase A and acid hydrolases. The nonpreformed mediators include products of arachidonic acid, prostaglandin D2, leukotrienes C4 and B4 and platelet activating factor. Another class of mediators, the cytokines are produced by mast cells upon IgE-mediated activation, or by other cells, including recruited Th2 lymphocytes. The cytokines are predominantly responsible for the late phase reaction which begins two to four hours after elicitation of many immediate hypersensitivity reactions. One cytokine, tumor necrosis factor alpha, may exist in the mast cells as preformed stores, or may represent a newly synthesized product released over a period of hours.

It should be noted that the composition of the invention leads to inhibition or decrease in mast cell activation or degranulation. Thus, the term “decrease in mast cell activation” refers to decrease, attenuation, reduction in cell degranulation and mediator secretion and release. In some particular embodiments, the composition of the invention leads to decrease of secretion or release of one or more of the following: serine proteases such as tryptase, histamine (2-5 pg/cell), serotonin, proteoglycans, mainly heparin (active as anticoagulant), Granzyme B, VEGF; newly formed lipid mediators (eicosanoids), for example, thromboxane, prostaglandin D2, leukotriene C4, platelet-activating factor, cytokines, Eosinophil chemotactic factor, IL-6, IL-4, IL-8 and TNF-α.

The terms “inhibition”, “decrease”, “moderation” or “attenuation” as referred to herein, relate to the retardation, restraining or reduction of a process, specifically the activation of mast cells, and/or release of mediator/s, by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%.

With regards to the above, it is to be understood that, where provided, percentage values such as, for example, 10%, 50%, 120%, 500%, etc., are interchangeable with “fold change” values, i.e., 0.1, 0.5, 1.2, 5, etc., respectively.

It should be appreciated that the definition of the term “inhibition” as disclosed herein is applicable for any embodiment or aspect of the invention.

It should be emphasized that the decrease in mast cell activation should be via a mechanism that does not involve mast cell death due to necrosis or apoptosis.

As noted above, the composition of the invention comprises active ingredient/s, specifically the mitochondrial-targeted STAT3 inhibitor/s in an amount sufficient for decreasing activation of MC, provided that nuclear functions of STAT3 are not affected. It should be noted that STAT3 nuclear functions include activities as a transcription factor, for example in inducing the expression of Bcl-xl, c-myc, Mcl-1. Therefore, as also shown by the Examples, the composition of the invention does not affect the expression of STAT3 induced genes.

In certain embodiments, the active ingredient present in the pharmaceutical composition of the invention in an amount effective for, sufficient, appropriate, required for, enabling, inhibiting and/or decreasing phosphorylation of Ser727 of STAT3. In more specific embodiments, the amount is effective for inhibiting and/or decreasing phosphorylation of Ser727 of STATS by Erk1/2. In more specific embodiments, Erk1/2 as used herein refers to extracellular-signal-regulated kinases (ERKs) or classical MAP kinases that are widely expressed protein kinase intracellular signalling molecules involved in functions including the regulation of meiosis, mitosis, and postmitotic functions in differentiated cells. It should be further noted that “extracellular-signal-regulated kinases”, is sometimes used as a synonym for mitogen-activated protein kinase (MAPK), but has more recently been adopted for a specific subset of the mammalian MAPK family.

In some embodiments, the active ingredient present in the pharmaceutical composition of the invention in an amount effective for reducing mitochondrial oxidative phosphorylation. In more specific embodiments, the term Oxidative phosphorylation (or OXPHOS in short) as used herein is the metabolic pathway in which the mitochondria in cells use their structure, enzymes, and energy released by the oxidation of nutrients to reform ATP.

In further embodiments, the active ingredient is present in the pharmaceutical composition of the invention in an amount effective for inhibiting and/or decreasing elevation in ATP levels and/or oxygen consumption, caused by activation of MC. In more specific embodiments, the active ingredient is present in an amount effective for reducing ATP levels and/or oxygen consumption. Still further embodiments relate to the composition of the invention wherein the active ingredients are present in an amount effective for inhibiting and/or decreasing cytochrome c reductase (complex 3) activity. The term cytochrome c reductase, as used herein, may be also referred to as oxidoreductase, cytochrome bc₁ complex, complex III or complex 3, is the third complex in the electron transport chain, playing a critical role in biochemical generation of ATP (oxidative phosphorylation). Complex III is a multisubunit transmembrane protein.

Other embodiments of the invention relate to the composition of the invention wherein the active ingredients are present in an amount effective for reduction in TNFalpha secretion and/or beta-hexosaminidase release. In further embodiments, the composition of the invention comprises active ingredients in an amount effective for reduction of the release of at least one of serine proteases such as tryptase, histamine (2-5 pg/cell), serotonin, proteoglycans, mainly heparin (active as anticoagulant), Granzyme B, VEGF; newly formed lipid mediators (eicosanoids), for example, thromboxane, prostaglandin D2, leukotriene C4, platelet-activating factor, cytokines, Eosinophil chemotactic factor, IL-6, IL-4 and IL-8 and TNF-α.

In yet further embodiments, the active ingredient may be present in the composition of the invention in an amount effective for inhibiting or decreasing apoptosis. In more specific embodiments, the compositions of the invention comprise the active ingredient in an amount effective to avoid apoptosis. In other embodiments, said amount is not sufficient to induce apoptosis. In some embodiments, the amount is effective to avoid, inhibit or decrease cleavage of caspase 3. In other embodiments, said amount is not sufficient to induce necrosis or any other cell death mechanism. It yet some other embodiment, the amount is effective for retaining viability of the cells.

Still further embodiments relate to active ingredient in an amount effective for inhibiting or decreasing MC activation (degranulation) within a time period of between about 1 second to about 72 hrs, about 1, 2, 3, 4, 5, 6, 7, 8 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 seconds, about 1, 2, 3, 4, 5, 6, 7, 8 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 minutes, about 1, 2, 3, 4, 5, 6, 7, 8 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71 or 72 hrs.

In some embodiments, the at least one mitochondrial targeting moiety of the composition of the invention may be at least one of a lipohilic cation and/or at least one mitochondrial targeting sequence. In some embodiments, the mitochondrial targeting moiety used for the composition of the invention may be a mitochondrial targeting peptide, specifically, the MTS peptides of SEQ ID NO. 8, as disclosed above.

In yet another embodiment, the mitochondrial targeting moiety used for the composition of the invention may be a lipophilic cation. In more specific embodiments, the lipophilic cation may be a phosphonium cation, an arsonium cation, an ammonium cation, flupritine, MKT-077, a pyridinium ceramide, a quinolium, a liposomal cation, a sorbitol guanidine, a cyclic guanidine or a rhodamine.

In further embodiments, the lipophilic cation of the composition of the invention may be Triphenylphosphonium (TPP) and the at least one STAT3 inhibiting moiety may be at least one of a small organic molecule based moiety, a nucleic acid based moiety, an amino acid based moiety and any combinations thereof.

It yet another embodiment, the STAT3 inhibiting moiety used for the composition of the invention may be a STAT3 inhibitor that inhibits phosphorylation of Ser727 of STAT3.

Still further embodiments relate to the composition of the invention comprising as at least one STAT3 inhibiting moiety any one of STAT3 Inhibitor V, 6-Nitrobenzo[b]thiophene 1,1-dioxide (Stattic); (1E,6E)-1,7-Bis(4-hydroxy-3-methoxyphenyl)-1,6-heptadiene-3,5-dione (curcumin); STAT3 Inhibitor XIII, C188-9; Protein Inhibitor Of Activated STAT, 3 (PIAS3), any derivatives, analogs or any combinations thereof or any vehicle, matrix, nano- or micro-particle comprising the same.

In yet some other embodiments, the mitochondrial-targeted STAT3 inhibitor used for the composition of the invention may further comprise at least one mast cell (MC) targeting moiety.

In some embodiments, the composition of the invention may comprise 6-Nitrobenzo[b]thiophene 1,1-dioxide associated or conjugated with at least one TPP moiety.

In other embodiments, the composition of the invention may comprise (1E,6E)-1,7-Bis(4-hydroxy-3-methoxyphenyl)-1,6-heptadiene-3,5-dione or any derivatives thereof associated with TPP. In more specific embodiments, the composition may comprise (1E,6E)-1,7-Bis(4-hydroxy-3-methoxyphenyl)-1,6-heptadiene-3,5-dione or any derivative/s, salt/s or ester/s thereof associated with TPP. The association between (1E,6E)-1,7-Bis(4-hydroxy-3-methoxyphenyl)-1,6-heptadiene-3,5-dione may be with two TPP moieties. It should be noted that said compound is referred to herein and in the following Examples as mito-cur-1 (or Mitocur-1).

In some other embodiments, the composition may comprise (1E,6E)-1,7-bis(4-hydroxyphenyl) hepta-1,6-diene-3,5-dione associated with TPP. The association between (1E,6E)-1,7-bis(4-hydroxyphenyl) hepta-1,6-diene-3,5-dione may be with two TPP moieties. It should be noted that said compound is referred to herein and in the following Examples as mito-cur-3 (or Mitocur-3).

In certain embodiments, the STAT3 inhibiting moiety may be encapsulated in a nano- or micro-particle. In more specific embodiments, the vehicle, matrix, nano- or micro-particle comprises a STAT3 inhibiting moiety and at least one mitochondrial targeting moiety.

In some embodiments, the composition of the invention may comprise liposomes encapsulating in their intraliposomal core at least one STAT3 Inhibitor V, 6-Nitrobenzo[b]thiophene 1,1-dioxide (Stattic) and having at least one TPP associated onto the outer liposomal surface. In some specific embodiments, the composition of the invention may comprise at least one stattic molecule encapsulated in a liposome. In more specific embodiments, such mitochondrial-targeted STAT3 inhibitor is referred to herein as Lipo-stattic or LipoS.

In certain embodiments, the composition of the invention may comprise at least one PIAS3 or any fragments or peptides thereof, said PIAS3 is being attached to at least one mitochondrial targeting peptide. In some specific embodiments, the PIAS3 may be attached or fused to at least one MTS. In some specific embodiments, said MTS may comprise the amino acid sequence as denoted by SEQ ID NO. 8. In yet further embodiments, the PIAS3 may comprise the amino acid sequence as denoted by SEQ ID NO. 16 (referred to herein as PIAS3-mito).

In certain embodiments, the pharmaceutical composition of the invention comprises an active ingredient in an amount effective for inhibiting and/or decreasing MC activation in a subject in need thereof.

The composition of the invention may be used for inhibiting and/or decreasing MC activation and associated disorders that include allergic reactions and specifically, respiratory allergic conditions such as asthma.

Therefore, according to specific embodiments, the composition of the invention may be adapted for pulmonary delivery. In more specific embodiments, such pulmonary delivery may be affected using nasal or oral administration, or any combination thereof.

The term “pharmaceutical composition” in the context of the invention means that the composition is of a grade and purity suitable for therapeutic administration to human subjects and is present together with at least one of carrier/s, diluent/s, excipient/s and/or additive/s that are pharmaceutically acceptable. The pharmaceutical composition may be suitable for any mode of administration whether oral or parenteral, by injection or by topical administration by inhalation, intranasal spray or intraocular drops.

Thus, some embodiments consider the composition's according to the invention, particularly for treating respiratory diseases such as asthma. According to one embodiment, such combined composition may be particularly adapted for pulmonary delivery by oral or nasal inhalation. More specifically, pulmonary delivery may require the use of liquid nebulizers, aerosol-based metered dose inhalers (MDI's), or dry powder dispersion devices. Still further, it should not be overlooked that the composition of the invention, particularly when used for treating allergic-skin disorders such as eczema, dermatitis, may be an acceptable topically applied composition as will be described in more detail herein after. Alternatively, the administration may be systemic such as by sublingual, rectal, vaginal, buccal, parenteral, intravenous, intramuscular, subcutaneous modes transdermal, inrtaperitoneal or intranasal modes of administration. However, oral, transmucosal, intestinal or parenteral delivery, including intramuscular, subcutaneous and intramedullary injections as well as rectal, intrathecal, direct intraventricular, intravenous, intraocular injections or any other medically acceptable methods of administration can be considered as well.

The compositions of the invention may comprise carriers suitable for pulmonary delivery that may involve nasal and/or oral administration. In specific embodiments, such carrier may be any one of spray, mist, patch, foam, alcoholic foam, oily foam, aqueous foam, bandage, membrane, gel, cream, emulsion, oily solution, aqueous solution, hydroethanolic solution, hydro-alcoholic-glycolic solution, mixture of alcohol and glycols, microemulsion, double emulsions, nanoemulsion, nanoparticles, microparticles, microcapsules, lipid particles, lipospheres, liposomes, lipid vesicles, solid lipid nanoparticles, liquid crystals, eutectic mixtures, eutectic crystsls, cubosomes, hexazomes, micelosomes, liposomal systems, vesicular systems, nanocubes, ethosomes, hydroethanolic systems, mixtures of alcohols and glycols, aqueous mixtures of alcohols and glycols, buffer solutions, polymer based delivery systems, hydrophilic or lipophilic suppository bases, chitosan and derivatives bases. For nasal administration, suitable carriers are preferably water-soluble and include water, propylene glycol and other pharmaceutically acceptable alcohols, xanthan gum, locust bean gum, galactose, other saccharides, oligosaccharides and/or polysaccharides, starch, starch fragments, dextrins, British gum and mixtures thereof. For buccal administration, suitable carriers are water-soluble carrier materials, for example (poly)saccharides like hydrolysed dextran, dextrin, mannitol, and alginates, or mixtures thereof, or mixtures thereof with other carrier materials like polyvinylalcohol, polyvinylpyrrolidine and water-soluble cellulose derivatives, like hydroxypropyl cellulose. In specific embodiments, the buccal carrier material may be gelatin, especially partially hydrolysed gelatin.

Pharmaceutical formulations adapted for nasal administration wherein the carrier is a solid include a coarse powder having a particle size for example in the range 20 to 500 microns which is administered in the manner in which snuff is taken, i.e. by rapid inhalation through the nasal passage from a container of the powder held close up to the nose. Suitable formulations wherein the carrier is a liquid, for administration as a nasal spray or as nasal drops, include aqueous or oil solutions of the active ingredient. Suitable formulations wherein a semisolid carriers such as gel, cream or ointment may be also used for nasal administration according to the invention.

As indicated herein above, the mitochondrial-targeted STATS inhibitor, specifically, stattic-TPP (MitoS), Lipo-stattic (LipoS), Curcumin-TPP (Mitocur-1, and Mitocur-3), or any salt, base, ester or amide thereof, or PIAS3-mito, used by the invention can be administered via an administration device suitable for nasal administration. As used herein, an administration device is any pharmaceutically acceptable device adapted to deliver a composition of the invention to a subject's nose. A nasal administration device can be a metered administration device (metered volume, metered dose, or metered-weight) or a continuous (or substantially continuous) aerosol-producing device. Suitable nasal administration devices also include devices that can be adapted or modified for nasal administration. In some embodiments, the nasally administered dose can be absorbed into the bloodstream of a subject.

A metered nasal administration device delivers a fixed (metered) volume or amount (dose) of a nasal composition upon each actuation. Exemplary metered dose devices for nasal administration include, by way of example and without limitation, an atomizer, sprayer, dropper, squeeze tube, squeeze-type spray bottle, pipette, ampule, nasal cannula, metered dose device, nasal spray inhaler, breath actuated bi-directional delivery device, pump spray, pre-compression metered dose spray pump, monospray pump, bispray pump, and pressurized metered dose device. The administration device can be a single-dose disposable device, single-dose reusable device, multi-dose disposable device or multi-dose reusable device.

The mitochondrial-targeted STAT3 inhibitor or specifically, stattic-TPP, Lipo-stattic, Curcumin-TPP, PIAS3-mito or compositions thereof according to the invention can be used with any known metered administration device. In some embodiments, the device is a pump nasal spray or a squeeze bottle.

A continuous aerosol-producing device delivers a mist or aerosol comprising droplet of a nasal composition dispersed in a continuous gas phase (such as air). A nebulizer, pulsating aerosol nebulizer, and a nasal continuous positive air pressure device are exemplary of such a device. Suitable nebulizers include, by way of example and without limitation, an air driven jet nebulizer, ultrasonic nebulizer, capillary nebulizer, electromagnetic nebulizer, pulsating membrane nebulizer, pulsating plate (disc) nebulizer, pulsating/vibrating mesh nebulizer, vibrating plate nebulizer, a nebulizer comprising a vibration generator and an aqueous chamber, a nebulizer comprising a nozzle array, and nebulizers that extrude a liquid formulation through a self-contained nozzle array.

In some embodiments, the nasal device is a nebulizer for nasal administration. The size of the reservoir varies from one type of nebulizer to another. The volume of the liquid formulation can be adjusted as needed to provide the required volume for loading into the reservoir of a particular type or brand of nebulizer. The volume can be adjusted by adding additional liquid carrier. In general, the reservoir volume of a nebulizer is about 10 μl to 100 mL. Low volume nebulizers, such as ultrasonic and vibrating mesh/vibrating plate/vibrating cone/vibrating membrane nebulizers, pre-filled reservoir strips inclusive of delivery nozzle typically have a reservoir volume of 10 μl to 6 mL or 10 μl to 5 mL. The low volume nebulizers provide the advantage of shorter administration times as compared to large volume nebulizers.

The parameters used to effect nebulization via an electronic nebulizer, such as flow rate, mesh membrane size, aerosol inhalation chamber size, mask size and materials, inlet and outlet valves, outflow tube, internal channel plurality of air outputs communicating with the internal chamber, vibration generator and power source may be varied in accordance with the principles of the present invention to maximize their use with different types of aqueous mitochondrial-targeted STAT3 inhibitor or stattic-TPP, Lipo-stattic, Curcumin-TPP or PIAS3-mito compositions. In some embodiments, substantially all of a dose (weight or volume) is delivered in less than 1.5 minutes or continuously delivered over 1.5 to 60 minutes.

The administration devices can be equipped with different types of baffles, valves, tubes, channels, reservoirs, mixing chambers, vortex chamber, particle dispersion chamber, nasal adapter, vibrating pulse and/or sound wave generator.

Nebulizers that nebulize liquid formulations containing no propellant are suitable for nasal delivery of stattic-TPP, Lipo-stattic, Curcumin-TPP provided herein. Any of these and other known nebulizers can be used to deliver the formulation of the invention including but not limited to the following: nebulizers available from Pari GmbH (Starnberg, Germany), DeVilbiss Healthcare (Heston, Middlesex, UK), Healthdyne, Vital Signs, Baxter, Allied Health Care, Invacare, Hudson, Omron, Bremed, AirSep, Luminscope, Medisana, Siemens, Aerogen, Mountain Medical, Aerosol Medical Ltd. (Colchester, Essex, UK), AFP Medical (Rugby, Warwickshire, UK), Bard Ltd. (Sunderland, UK), Carri-Med. Ltd. (Dorking, UK), Plaem Nuiva (Brescia, Italy), Henleys Medical Supplies (London, UK), Intersurgical (Berkshire, UK), Lifecare Hospital Supplies (Leies, UK), Medic-Aid Ltd. (West Sussex, UK), Medix Ltd. (Essex, UK), Sinclair Medical Ltd. (Surrey, UK), and many other companies.

Nebulizers for use herein include, but are not limited to, jet nebulizers (optionally sold with compressors), ultrasonic nebulizers, vibrating membrane, vibrating mesh nebulizers, vibrating plate nebulizers, vibrating cone nebulizer, and others. Exemplary jet nebulizers for use herein include Pari LC plus/ProNeb, Pari LC plus/ProNeb Turbo, Pari LC Plus/Dura Neb 1000 & 2000 Pari LC plus/Walkhaler, Pari LC plus/Pari Master, Pari LC star, Omron CompAir XL Portable Nebulizer System (NE-C18 and JetAir Disposable nebulizer), Omron compare Elite Compressor Nebulizer System (NE-C21 and Elite Air Reusable Nebulizer, Pari LC Plus or Pari LC Star nebulizer with Proneb Ultra compressor, Pulomo-aide, Pulmo-aide LT, Pulmo-aide traveler, Invacare Passport, Inspiration Healthdyne 626, Pulmo-Neb Traverler, DeVilbiss 646, Whisper Jet, Acorn II, Misty-Neb, Allied aerosol, Schuco Home Care, Lexan Plasic Pocet Neb, SideStream Hand Held Neb, Mobil Mist, Up-Draft, Up-Draft II, T Up-Draft, ISO-NEB, Ava-Neb, Micro Mist, and PulmoMate. Exemplary ultrasonic nebulizers for use herein include MicroAir, UltraAir, Siemens Ultra Nebulizer 145, CompAir, Pulmosonic, Scout, 5003 Ultrasonic Neb, 5110 Ultrasonic Neb, 5004 Desk Ultrasonic Nebulizer, Mystique Ultrasonic, Lumiscope's Ultrasonic Nebulizer, Medisana Ultrasonic Nebulizer, Microstat Ultrasonic Nebulizer, and Mabismist Hand Held Ultrasonic Nebulizer. Other nebulizers for use herein include 5000 Electromagnetic Neb, 5001 Electromagnetic Neb 5002 Rotary Piston Neb, Lumineb I Piston Nebulizer 5500, Aeroneb Portable Nebulizer System, Aerodose® Inhaler, and AeroEclipse Breath Actuated Nebulizer.

The volume or amount of composition administered can vary according to the intended delivery target and administration device used. The amount of active agent in a dose or unit dose can vary according to the intended delivery target and administration device used.

It should be recognized that in addition to the systems described above, any additional nasal delivery modes or systems facilitating nasal delivery of mitochondrial-targeted STAT3 inhibitors for treating and preventing conditions associates with activation of MC, specifically, allergic reactions, is encompassed by the present invention.

It should be noted that the invention further contemplates the use of at least one mitochondrial-targeted STAT3 inhibitors, specifically, stattic-TPP, Lipo-stattic, Curcumin-TPP or PIAS3-mito for combined nasal and oral or buccal or sublingual administration. In such particular embodiments, the stattic-TPP, Lipo-stattic, Curcumin-TPP or PIAS3-mito used by the invention may be administered first using the oral route, for a period of about 1 day to 1 month, followed by repetitive nasal administrations of stattic-TPP, Lipo-stattic, Curcumin-TPP or PIAS3-mito. The combined mode of administration is described in more detail hereinafter.

Furthermore, the STAT3 inhibitor or any composition thereof may be administered as a single daily dose or multiple daily doses, preferably, every 1 to 7 days. It is specifically contemplated that administration may be carried out once, twice, thrice, four times, five times or six times daily, or may be performed once daily, once every 2 days, once every 3 days, once every 4 days, once every 5 days, once every 6 days, once every week, two weeks, three weeks, four weeks or even a month. The treatment may last up to a day, two days, three days, four days, five days, six days, a week, two weeks, three weeks, four weeks, a month, two months three months or even more. Specifically, administration will last from one day to one month. Most specifically, administration will last from one day to 7 days.

According to certain embodiments, a mitochondrialy-targeted STAT3 inhibitor nasal administration system, specifically a stattic-TPP, Lipo-stattic, Curcumin-TPP or PIAS3-mito nasal administration system is provided by the present invention. The nasal administration system of the invention allows multiple daily administrations, ease of administration and rapid delivery. In order to be efficiently absorbed by the subject, and to preserve the active ingredient, a carrier for the active ingredient may be necessary.

Thus, particular embodiments according to the invention contemplate the use wherein the at least one STAT3 inhibitor in the composition may be for example, stattic-TPP, Lipo-stattic, Curcumin-TPP, and wherein said mitochondrialy-targeted STAT3 inhibitor is administered intra-nasally, each intranasal application comprising for example, stattic-TPP, Lipo-stattic, Curcumin-TPP or PIAS3-mito or its salts incorporated in pharmaceutically acceptable carrier.

It is understood that certain embodiments of the invention encompass the use of at least one mitochondrialy-targeted STAT3 inhibitor, specifically, stattic-TPP (MitoS), Lipo-stattic (LipoS), Curcumin-TPP (Mitocur-1, and Mitocur-3), or any salt, base, ester or amide thereof or any mixture thereof, or PIAS3-mito, for nasal delivery, at a dose of between about 0.01 mg to 1000 mg. Specifically, between about 0.1 mg to about 900 mg, about 0.1 mg to about 800 mg, about 0.1 mg to about 700 mg, about 0.1 mg to about 600 mg, about 0.1 mg to about 500 mg, about 0.1 mg to about 400 mg, about 0.1 mg to about 300 mg, about 0.1 mg to about 200 mg, about 0.1 mg to about 100 mg, specifically about 0.1 mg to about 450 mg, about 0.1 mg to about 400 mg, about 0.1 mg to about 350 mg or about 0.1 mg to about 300 mg, about 0.1 mg to about 250 mg or about 0.1 mg to about 200 mg, most specifically, 0.1 mg to about 500 mg.

Other specific embodiments consider the nasal administration of the mitochondrialy-targeted STAT3 inhibitors, specifically stattic-TPP, Lipo-stattic, Curcumin-TPP or any salts or esters thereof, or PIAS3-mito, in doses of between about 0.1 mg to about 500 mg each administration once to ten times daily. More specifically, the stattic-TPP, Lipo-stattic, Curcumin-TPP or PIAS3-mito used by the invention may be in a dose of between about 0.1 to about 100 mg, specifically 0.1, 1, 10, 100, 150, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490 and 500 mg. According to certain embodiments, such dose may be administered once to ten times daily. Alternatively, administration may include a single daily dose of about 200 to about 500 mg. More specifically, about 0.1, 1, 10, 100, 150, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490 and 500 mg as a single dose

Single or multiple administrations of the compositions of the invention are administered depending on the dosage and frequency as required and tolerated by the patient. In any event, the composition should provide a sufficient quantity of the mitochondrial-targeted STAT3 inhibitor/s of the invention to effectively treat the patient. Preferably, the dosage is administered once but may be applied periodically until either a therapeutic result is achieved or until side effects warrant discontinuation of therapy. Generally, the dose is sufficient to treat or ameliorate symptoms or signs of disease without producing unacceptable toxicity to the patient.

Specific embodiments contemplate skin inflammatory conditions, specifically, eczema, and dermatitis, therefore, treatment by topical administration of the affected skin areas of an ointment, cream, suspensions, paste, lotions, powders, solutions, oils, encapsulated gel, liposomes containing the mitochondrial-targeted STAT3 inhibitor/s, any nano-particles containing the mitochondrial-targeted STAT3 inhibitor/s of the invention, or sprayable aerosol or vapors containing a combination of these inhibitors, are also encompassed by the invention. Conventional pharmaceutical carriers, aqueous, powder or oily bases, thickeners and the like may be necessary or desirable. The term “topically applied” or “topically administered” means that the ointment, cream, emollient, balm, lotion, solution, salve, unguent, or any other pharmaceutical form is applied to some or all of that portion of the skin of the patient skin that is, or has been, affected by, or shows, or has shown, one or more symptoms of dermatitis or eczema, or any other allergic disorders involving the skin.

It should be noted that in certain embodiments, a topical application of the mitochondrial-targeted STAT3 inhibitor/s by the method of the invention particularly in treating allergic-skin, any transdermal delivery may be used. As used herein, the term “transdermal” refers to delivery, administration or application of a drug by means of direct contact with tissue, such as skin or mucosa. Such delivery, administration or application is also known as percutaneous, dermal, transmucosal and buccal.

Therapeutic compositions for transdermal administration, or “dermal compositions” are compositions which contain one or more drugs solubilized therein, specifically, any of the mitochondrial-targeted STAT3 inhibitor/s or combinations thereof according to the invention. The composition is applied to a dermal area, for dermal administration or topical application of the drugs. Such a dermal composition may comprise a polymer matrix with the one or more drugs contained therein. The polymer matrix may be a pressure-sensitive adhesive for direct attachment to a user's (e.g., a patient's) skin. Alternatively, the polymer matrix may be non-adhesive and may be provided with separate adhesion means (such as a separate adhesive layer) for adhering the composition to the user's skin.

As used herein, “matrix” is defined as a polymer composition which incorporates a therapeutically effective amount of the drug therein. The matrix may be monolithic and comprise a pressure-sensitive adhesive, or it may use separate attachment means for adhering or holding to the user's skin, such as a separate adhesive layer. A dermal drug delivery system comprising a matrix may optionally include additional drug supply means for continuously replenishing the drug supply in the matrix. As used herein, a polymer is an “adhesive” if it has the properties of an adhesive per se, or if it functions as an adhesive by the addition of tackifiers, plasticizers, cross-linking agents or other additives.

Thus, the invention also contemplates the use according to the invention, wherein the at least one pharmaceutically acceptable carrier is adapted for transdermal administration, and the carrier may further comprise at least one agent for enhancing penetration through the skin. The term “skin” as used herein refers to the outer covering of a mammal body, comprising the epidermis and the dermis. More specifically, “skin” as used herein means the air-contacting part of the human body, to a depth of about 7 mm from the air interface; as such, it also includes the nails.

According to certain embodiments, an agent for enhancing penetration through the skin used by the invention may be used. Such agent may include any one of terpens, unsaturated acids, oleic acid, azone derivatives, surfactants, cetomacrogol, short chain alcohols, glycols, sulphoxides, alkyl sulphoxides, urea, sunscreen molecules, sunscreens in ethanolic solutions, short chain alcohols, glycols, or any combination thereof, vesicular carriers such are niosomes, flexible vesicles, transfersomes, ethosomes.

As used herein, the term “permeation enhancement” refers to an increase in the permeability of skin to a therapeutic agent, specifically mitochondrial-targeted STAT3 inhibitor/s or specifically, stattic-TPP (MitoS), Lipo-stattic (LipoS), Curcumin-TPP (Mitocur-1, and Mitocur-3), or any salt, base, ester or amide thereof or PIAS3-mito in the presence of a permeation enhancer as compared to permeability of skin to the same therapeutic agent in the absence of a permeation enhancer. An increase in the permeation may range between about 10-99.9%, about 20 to 80%, about 30 to 70%, about 40 to 60% and about 50% or more. More specifically, about 10, 20, 30, 40, 50, 60, 70, 80, 90, 95 and 99.9%. In other embodiments an increase in permeation enhancement often may be much more than 100%, it could range from many times to two orders of magnitude or even more. Moreover, with regards to the above, it is to be understood that, where provided, percentage values such as, for example, 10%, 50%, 120%, 500%, 1000%, 2000% etc., are interchangeable with “fold change” values, i.e., 0.1, 0.5, 1.2, 5, etc., respectively.

According to certain embodiments, the drug, specifically, at least one mitochondrial-targeted STAT3 inhibitor/s of the invention, may be administered transdermally for the treatment of disorders associated with activation of MC, specifically, allergic conditions by means such as iontophoresis, phonophoresis, microneedles, jet pressure, microporation, radioporation, heat, stripping the stratum corneum, using radiowaves, or a combination of any of the above.

In certain embodiments, transdermal delivery involves the use of a transdermal patch which adheres to the skin. A transdermal patch has several components like liners, adherents, drug reservoirs, drug release membrane etc. which play a vital role in the release of the drug via skin. Various types of patches along with various methods of applications have been discovered to deliver the drug from the transdermal patch.

The application of the transdermal patch and the flow of the active drug constituent from the patch to the circulatory system via skin occur through various methods described herein, involving active or passive delivery.

Mitochondrialy targeted STAT3 inhibitors can also be formulated for oral delivery. Oral solid dosage forms are known to those skilled in the art. Solid dosage forms include tablets, capsules, pills, troches or lozenges, cachets, pellets, powders, or granules or incorporation of the material into particulate preparations of polymeric compounds such as polylactic acid, polyglycolic acid, etc. or into liposomes. Such compositions may influence the physical state, stability, rate of in vivo release, and rate of in vivo clearance of the present proteins and derivatives.

Depending on the clinical context and on the route of administration, the compositions prepared according to the invention can inhibit IgE- and optionally also IgG-induced mast cell activation. They can hence be used to prevent, alleviate and/or treat any inflammatory manifestation implying mast cell activation by antibodies in the presence of antigens. In particular, when the IgE-induced activation is inhibited, these compositions can be used for preventing, alleviating and/or treating an allergy or allergic manifestations. The allergies considered herein are caused by IgE antibodies which bind to mast cells and, when recognizing specific antigens, trigger their activation Importantly, these compositions can be used to prevent, treat or alleviate allergic manifestations (e.g., asthma, rhinitis or hay fever, allergic eczema, anaphylactic shock etc.), even in subjects who have already been sensitized to an antigen, and who have already been diagnosed as allergic to this antigen. For example, a person who has suffered for many years from hay fever can prevent the reappearance of the symptoms by taking compositions prepared according to the invention. A huge number of antigens can cause allergies, which can manifest themselves in a great variety of clinical symptoms. Non-limitative examples of antigens frequently at the origin of allergies are environmental allergens such as mite, cockroach antigens, birch pollen, grass pollen, animal hair dander antigens, bee venom, or food allergens such as milks, peanut, shrimp, soya, eggs, cereal products, fruits, etc. Depending on the context and the individual, the clinical symptoms can be local (which is the case, for example, in allergic rhinitis, conjunctivitis or otitis), regional (e.g., asthma, dermatitis, gastroenterological problems and Quincke's oedema), or general (e.g., anaphylactic shock). Some pathologies are sometimes abusively defined as allergies, although they do not depend on the above-recalled mechanism. This is the case, for example, of delayed-type hypersensitivity reactions.

Thus, in a further aspect, the invention provides a method for inhibiting, reducing or decreasing MC activation, the method comprising contacting mast cells with an effective amount of at last one STAT3 inhibiting moiety or any vehicle, matrix, nano- or micro-particle comprising the same, associated with at least one mitochondrial targeting moiety, or with any composition comprising the same.

In certain embodiments, the invention provides methods for decreasing mast cell activation in a subject in need thereof. More specifically, the method comprises administering the subject an effective amount of at last one STAT3 inhibiting moiety or any vehicle, matrix, nano- or micro-particle comprising the same, associated with at least one mitochondrial targeting moiety, or with any composition comprising the same.

Still further, in some embodiments the at least one mitochondrial targeting moiety may be at least one of a lipohilic cation and/or at least one mitochondrial targeting sequence, and said at least one STAT3 inhibiting moiety is at least one of a small molecule based moiety, a nucleic acid based moiety, an amino acid based moiety and any combinations thereof.

In some embodiments, the at least one STAT3 inhibiting moiety used by the method of the invention may be any one of STAT3 Inhibitor V, 6-Nitrobenzo[b]thiophene 1,1-dioxide (Stattic); (1E,6E)-1,7-Bis(4-hydroxy-3-methoxyphenyl)-1,6-heptadiene-3,5-dione (curcumin); STAT3 Inhibitor XIII, C188-9; Protein Inhibitor Of Activated STAT, 3 (PIAS3), any derivatives, analogs or any combinations thereof or any vehicle, matrix, nano- or micro-particle comprising the same.

In some embodiments, the at least one STAT3 inhibiting moiety used by the method of the invention may be 6-Nitrobenzo[b]thiophene 1,1-dioxide associated with TPP. In certain embodiments, such mitochondrial targeted STAT3 inhibitor is referred to herein as MitoS.

In other embodiments, the at least one STAT3 inhibiting moiety used by the method of the invention may be (1E,6E)-1,7-Bis(4-hydroxy-3-methoxyphenyl)-1,6-heptadiene-3,5-dione or derivatives thereof associated with TPP. In some specific embodiments, the mitochondrial targeted STAT3 inhibitor used by the method of the invention may be at least one 1E,6E)-1,7-Bis(4-hydroxy-3-methoxyphenyl)-1,6-heptadiene-3,5-dione or any derivative/s, salt/s or ester/s thereof associated with TPP. The association between (1E,6E)-1,7-Bis(4-hydroxy-3-methoxyphenyl)-1,6-heptadiene-3,5-dione may be with two TPP moieties. It should be noted that said compound is referred to herein and in the following Examples as mito-cur-1 (or Mitocur-1).

In some other embodiments, the mitochondrial targeted STAT3 inhibitor used by the method of the invention may be (1E,6E)-1,7-bis(4-hydroxyphenyl) hepta-1,6-diene-3,5-dione associated with TPP. The association between (1E,6E)-1,7-bis(4-hydroxyphenyl) hepta-1,6-diene-3,5-dione may be with two TPP moieties. It should be noted that said compound is referred to herein and in the following Examples as mito-cur-3 (or Mitocur-3).

In certain embodiments, the STAT3 inhibiting moiety may be encapsulated in a nano- or micro-particle. In other embodiments, the at least one STAT3 inhibiting moiety used by the method of the invention may be vehicle, matrix, nano- or micro-particle that comprise a STAT3 inhibiting moiety and at least one mitochondrial targeting moiety.

In some embodiments, the method of the invention may use liposomes encapsulating in their intraliposomal core at least one 6-Nitrobenzo[b]thiophene 1,1-dioxide and having at least one TPP associated onto the outer liposomal surface. It should be noted that said inhibitor is referred to herein as LipoS.

In yet another embodiment, the method of the invention may use at least one PIAS3 molecule or any fragments and peptides thereof, that may be preferably, attached to at least one MTS that comprises Met Ser Val Leu Thr Pro Leu Leu Leu Arg Gly Leu Thr Gly Ser Ala Arg Arg Leu Pro Val Pro Arg Ala Lys Ile His Ser Leu, as denoted by SEQ ID NO. 8.

This invention thus provides anti-allergy agents and, more particularly, relates to novel compounds, formulations and methods for the prophylaxis and treatment of allergy and pulmonary disorders. The invention particularly relates to compositions and methods that are efficacious for the treatment of mast cell mediated inflammatory disorders.

Thus, in a further aspect, the invention provides a method for treating a subject suffering from a medical condition associated with or induced by mast cell activation. In more specific embodiments, the method of the invention may comprise administering to the subject a therapeutically effective amount of at last one STATS inhibiting moiety or any vehicle, matrix, nano- or micro-particle comprising the same, associated with at least one mitochondrial targeting moiety, or with any composition comprising the same according to the invention.

In certain embodiments, the methods of the invention are particularly applicable for treating a disorder characterized by undesirable release of mediator from immunologically stimulated mast cells.

Mediators released from human mast cells are central to the pathophysiology of allergy, asthma and anaphylaxis. In particular, mast cells and their release of histamine and other mediators play an important role in the symptomatology of asthma and other human diseases. During the early phase of human lung hypersensitivity reactions upon exposure to antigen (i.e., pollens, cats, etc.), mast cells release and are the major source of histamine, and newly synthesized lipid products of arachidonic acid metabolism: prostaglandin D2 and leukotriene C4. These mediators produce immediate breathlessness, which subsides in one hour but returns within 2-4 hours (the “late phase” response).

More specifically, as used herein, the phrase “a disease induced by activation of MCs” refers to a disease in which onset or progression of pathology is attributed at least in part to increase in activation of MCs. Non-limiting examples of diseases induced by activation of MCs include allergy, asthma, allergic rhinitis, allergic conjunctivitis, atopic dermatitis and atopic eczema, allergic disorders and responses to various allergens, systemic anaphylaxis, anaphylactic response to contrast media, anaphylactic response to muscle relaxants, systemic mastocytosis, mast cell activation syndrome (MAS), morphea/urticaria pigmentosa, mast cell leukemia, atherosclerosis, graft rejection, multiple sclerosis, fibrotic lung diseases, neurofibromatosis, keloids, scleroderma, acute gout, ocular cicatricial pemphigoid, inflammatory disease of the gut such as inflammatory bowel disease, colitis, interstitial cystitis, irritable bowel syndrome, and celiac disease and Crohn's disease, peritoneal adhesions, chronic graft versus host disease (GVHD), extrinsic bronchial asthma, nasal polyposis, Wegener's granulomatosis, intrinsic bronchial asthma, interstitial and other pulmonary diseases, hypersensitivity pneumonitis, allergic bronchopulmonary aspergillosis, sarcoidosis, idiopathic pulmonary fibrosis, toxocariasis, filariasis, schistosomiasis, trichinosis, neoplastic and myeloproliferative diseases, T cell lymphomas, Hodgkin's disease, psoriasis, diabetes mellitus, systemic lupus erythematosus (SLE), Sjogren's syndrome, rheumatoid arthritis, rheumatoid osteoarthritis, polymyositis, autoimmune thyroid disease, autoimmune gastritis and pernicious anemia, and autoimmune hepatitis.

In certain embodiments, the methods of the invention may be applicable for medical condition associated with or induced by mast cell activation. Such condition may be for example, any one of allergic reaction, asthma, eczema, dermatitis, allergic rhinitis allergic conjunctivitis, anaphylaxis, mastocytosis and mast cell tumors.

In some embodiments, the invention provides methods for treating, preventing, ameliorating and inhibiting asthma. Asthma is a common chronic inflammatory disease of the airways characterized by variable and recurring symptoms, airflow obstruction, and bronchospasm. Symptoms include wheezing, coughing, chest tightness, and shortness of breath. Clinically, asthma is recognized by airway hyperactivity and reversible airways obstruction. Pathological derangements at the tissue level include constriction of airway smooth muscle, increased vascular permeability resulting in edema of airways, outpouring of mucus from goblet cells and mucus glands, parasympathetic nervous system activation, denudationofairway epithelial lining cells, and influx of inflammatory cells. Underlying these tissue effects are direct effects of potent mediators secreted following physical, inflammatory, or immunological activation and degranulation. The early phase of the asthmatic reaction is mediated by histamine and other mast cell mediators that induce rapid effects on target organs, particularly smooth muscle. The pathophysiologic sequence of asthma may be initiated by mast cell activation in response to allergen binding to IgE.

Asthma is clinically classified according to the frequency of symptoms, forced expiratory volume in one second (FEV1), and peak expiratory flow rate. Asthma may also be classified as atopic (extrinsic) or non-atopic (intrinsic), based on whether symptoms are precipitated by allergens (atopic) or not (non-atopic).

Asthma is controlled by environmental and genetic factors. These factors influence how severe asthma is and how well it responds to medication. The interaction is complex and not fully understood.

Prevention of the development of asthma is different from prevention of asthma episodes. Aggressive treatment of mild allergy with immunotherapy has been shown to reduce the likelihood of asthma development. In controlling symptoms, the first step is establishing a plan of action to prevent episodes of asthma by avoiding triggers and allergens, regularly testing for lung function, and using preventive medications.

It should be appreciated that the compositions and methods of the invention may be applicable for treating asthma as quick-relief medications used to treat acute symptoms and as long-term control medications used to prevent further exacerbation.

It should be noted that medications for asthma and other reparatory-associated disorders are typically provided as metered-dose inhalers (MDIs) in combination with an asthma spacer or as a dry powder inhaler. The spacer is a plastic cylinder that mixes the medication with air, making it easier to receive a full dose of the drug. A nebulizer may also be used. Appropriate inhalers applicable for the methods of the present invention are described in detail herein before.

Some embodiments of the invention consider a treatment of a subject suffering from respiratory diseases, specifically asthma, with the mitochondrial-targeted STATS inhibitor/s of the invention, wherein the treatment results in the reduction of infiltration of inflammatory cells to the lungs, as judged by integer-graded histological sections evaluation, by about 5% to about 99.9%, specifically, about 5% to about 10%, about 10% to about 15%, about 15% to about 20%, about 20% to about 25%, about 25% to about 30%, about 35% to about 40%, about 40% to about 45%, about 45% to about 50%, about 50% to about 55%, about 55% to about 60%, about 65% to about 70%, about 75% to about 80%, about 80% to about 85%, about 85% to about 90%, about 90% to about 95% or about 95% to about 99.9%.

In other certain embodiments, the treatment results in the reduction of structural damage to the airway epithelium and goblet cell metaplasia and hyperplasia, as judged by integer-graded histological sections evaluation, by about 5% to about 99.9%, specifically, about 5% to about 10%, about 10% to about 15%, about 15% to about 20%, about 20% to about 25%, about 25% to about 30%, about 35% to about 40%, about 40% to about 45%, about 45% to about 50%, about 50% to about 55%, about 55% to about 60%, about 65% to about 70%, about 75% to about 80%, about 80% to about 85%, about 85% to about 90%, about 90% to about 95% or about 95% to about 99.9%.

In yet further embodiments, the treatment results in the reduction of mucus overproduction, as judged by integer-graded histological sections evaluation, by about 5% to about 99.9%, specifically, about 5% to about 10%, about 10% to about 15%, about 15% to about 20%, about 20% to about 25%, about 25% to about 30%, about 35% to about 40%, about 40% to about 45%, about 45% to about 50%, about 50% to about 55%, about 55% to about 60%, about 65% to about 70%, about 75% to about 80%, about 80% to about 85%, about 85% to about 90%, about 90% to about 95% or about 95% to about 99.9%.

In certain embodiments, wherein the treatment results in the reduction of scarring of the airways, by, by about 5% to about 99.9%, specifically, about 5% to about 10%, about 10% to about 15%, about 15% to about 20%, about 20% to about 25%, about 25% to about 30%, about 35% to about 40%, about 40% to about 45%, about 45% to about 50%, about 50% to about 55%, about 55% to about 60%, about 65% to about 70%, about 75% to about 80%, about 80% to about 85%, about 85% to about 90%, about 90% to about 95% or about 95% to about 99.9%.

In certain embodiments, the methods of the invention may be applicable for treating allergic rhinitis. Allergic rhinitis is a condition which results from the activation of mucosal mast cells beneath the nasal epithelium by allergens such as pollens. Allergic rhinitis is characterized by intense itching and sneezing, local oedema, nasal discharge and irritation of the nose as a result of histamine release. When caused by pollens of any plants, it is called pollinosis, and, if specifically caused by grass pollens, it is known as hay fever.

In still further embodiments, the method/s and composition/s provided by the invention may be used for treating allergic conjunctivitis. Allergic conjunctivitis is inflammation of the conjunctiva (the membrane covering the white part of the eye) due to allergy. Although allergens differ among patients, the most common cause is hay fever. Symptoms consist of redness (mainly due to vasodilation of the peripheral small blood vessels), oedema (swelling) of the conjunctiva, itching, and increased lacrimation (production of tears). If this is combined with rhinitis, the condition is termed allergic rhinoconjunctivitis. The symptoms are due to release of histamine and other active substances by mast cells, which stimulate dilation of blood vessels, irritate nerve endings, and increase secretion of tears.

As indicated above, the present invention contemplates methods for the treatment of different allergy-related respiratory diseases. In addition to asthma, such respiratory diseases may include any other acute allergy manifestations in airways, chronic rhinosinusitis (CRS), allergic rhinitis, COPD, nasal polyposis (NP), vasomotor rhinitis, airways hyper-responsiveness, or allergic sinusitis. The invention therefore provides methods and compositions for preventing, treating, ameliorating or inhibiting any of the respiratory diseases described above.

Thus, in certain embodiments, the invention provides methods and compositions for treating sinusitis. Sinusitis is inflammation of the paranasal sinuses, which may be due to infection, allergy or autoimmune issues.

Chronic sinusitis, by definition, lasts longer than three months and can be caused by many different diseases that share chronic inflammation of the sinuses as a common symptom. Chronic sinusitis cases are subdivided into cases with polyps and cases without polyps. When polyps are present, the condition is called chronic hyperplastic sinusitis; however, the causes are poorly understood and may include allergy, environmental factors such as dust or pollution, bacterial infection, or fungus (either allergic, infective, or reactive). Non-allergic factors, such as vasomotor rhinitis, can also cause chronic sinus problems.

Still further, the method/s and composition's of the invention may be used for treating Nasal polyps. Nasal polyps are polypoidal masses arising mainly from the mucous membranes of the nose and paranasal sinuses. They are overgrowths of the mucosa that frequently accompany allergic rhinitis. They are freely moveable and non-tender. Nasal polyps are usually classified into antrochoanal polyps and ethmoidal polyps. Antrochoanal polyps arise from the maxillary sinuses and are the much less common, ethmoidal polyps arise from the ethmoidal sinuses. Antrochoanal polyps are usually single and unilateral whereas ethmoidal polyps are multiple and bilateral.

Airway hyperresponsiveness (or other combinations with bronchial or hyperreactivity) is a state characterized by easily triggered bronchospasm (contraction of the bronchioles or small airways), and can be assessed with a bronchial challenge test. This most often uses products like metacholine or histamine These chemicals trigger bronchospasm in normal individuals as well, but people with bronchial hyperresponsiveness have a lower threshold. Bronchial hyperresponsiveness is a hallmark of asthma but also occurs frequently in people suffering from chronic obstructive pulmonary disease (COPD). Still further embodiments of the invention provide methods for treating hypersensitivity. Hypersensitivity reactions are the result of immune responses acting inappropriately and can be provoked by many antigens. They are produced by a combination of inflammatory mediators released by several cell types, resulting in acute inflammatory reaction with symptoms such as asthma or rhinitis. Airway inflammation is central to the pathogenesis of asthma and involves the recruitment and activation of mast cells, eosinophils, neutrophils, and lymphocytes into lung tissue and bronchoalveolar space.

Still further, the method of the invention may be suitable for allergic hypersensitivity. The inflammatory response characteristic of allergic or hypersensitivity reactions can be elicited by extrinsic antigens such as pollen, dust, food, dust-mites and chemicals in the environment.

There are four main classes of hypersensitivity reactions, which are distinguished by the type of immune cells and antibodies involved and the pathologies produced. In the most common IgE dependent allergic reactions, the inflammatory response involves mast cell degranulation, or emptying of the granules, triggered by allergen interaction with IgE molecules on the mast cell surface.

Inhaled allergens initiate respiratory allergies such as allergic rhinitis, hay fever and asthma, while ingested allergens may cause food allergies. Injected allergens, such as antibiotics and insect venoms, may cause life-threatening anaphylactic reactions.

Still further, it should be noted that the methods of the invention may be applicable for the treatment of anaphylaxis. Anaphylaxis is a serious allergic reaction that is rapid in onset and may cause death. It typically causes a number of symptoms including an itchy rash, throat swelling, and low blood pressure. Common causes include insect bites and stings, foods, and medications. More specifically, in anaphylaxis, body-wide degranulation of mast cells leads to vasodilation and, if severe, symptoms of life-threatening shock.

It should be also noted that the compositions and methods of the invention may be particularly useful in treating the underlying pathological changes in the airways associated with any of the diseases caused by MC activation, for example, basement membrane thickening, cell hypertrophy and hyperplasia, inflammatory cell influx, and other tissue remodeling.

Small quantities of mediators released from the cytoplasmic granules of activated mast cells on the mucosal surfaces of the respiratory system causes the symptoms associated with allergic rhinitis for example itchy watery eyes, runny nose, and sneezing. Many of the symptoms associated with asthma are directly associated with the effects of mediators released from the mast cell as well as indirectly through other cells that are recruited to the lung by these mediators. Larger amounts of these mediators contract the respiratory smooth muscles, limit breathing, and may cause an anaphylactic reaction which can be fatal. Release of these mediators in the skin causes edema, erythema, and wheal formation, i.e., urticaria. Urticaria is a skin condition characterized by the appearance of intensely itching wheals or welts with elevated centers and a surrounding area of erythema (redness). Wheals are usually distributed over the trunk and extremities of the body, but they may occur on any epithelial or mucosal surface. Urticaria may be acute, lasting six weeks or less, or chronic. A related skin condition, angiodema, with similar, but non-pruritic, sores, affects deeper levels of skin tissues. Other allergic skin disorders associated with activation of MCs will be disclosed herein after. It should be appreciated that the invention provides methods and compositions applicable for said disorders as well.

More specifically, certain embodiments of the invention relates to method of treating dermatitis. The term “dermatitis” refers to inflammation of the skin, in general. The different kinds usually have in common an allergic reaction to specific allergens. The term may be used to refer to eczema, which is also known as dermatitis eczema or eczematous dermatitis. A diagnosis of eczema often implies atopic dermatitis (childhood eczema), but without proper context, it means nothing more than a “rash”, i.e. a transient skin inflammation. In some embodiments, “dermatitis” and eczema are synonyms, while in other embodiments “dermatitis” implies an acute condition and “eczema” a chronic one, however, it should be noted that the two conditions are often classified together.

More specifically, dermatitis is characterized by itchy, erythematous, vesicular, weeping, and crusting patches. As noted above, the term eczema is also commonly used to describe atopic dermatitis or atopic eczema. The term eczema is broadly applied to a range of persistent skin conditions. These include dryness and recurring skin rashes that are characterized by one or more of these symptoms: redness, skin swelling, itching and dryness, crusting, flaking, blistering, cracking, oozing, or bleeding. Areas of temporary skin discoloration may appear and are sometimes due to healed injuries. Scratching open a healing lesion may result in scarring and may enlarge the rash.

Allergic-related skin disorder or allergic-skin conditions that may be treated by the compositions and methods of the invention may include also insect bites and stings. Insect bites occur when an insect is agitated and seeks to defend itself through its natural defense mechanisms, or when an insect seeks to feed off the bitten person. Insects inject formic acid, which can cause an immediate skin reaction often resulting in redness and swelling in the injured area. The sting from fire ants, bees, wasps and hornets are usually painful, and may stimulate a dangerous allergic reaction called anaphylaxis for at-risk patients, and some wasps can also have a powerful bite along with a sting. Bites from mosquitoes, fleas, and mites are more likely to cause itching than pain. The skin reaction to insect bites and stings usually lasts for up to a few days. However, in some cases the local reaction can last for up to two years. The reaction to a sting is of three types. The normal reaction involves the area around the bite with redness, itchiness, and pain. A large local reaction occurs when the area of swelling is greater than five cm. Systemic reactions are when symptoms occur in areas besides that of the bites.

In some embodiments, the invention provides a method for the treatment of mast cell tumor or mast cell cancer. As used herein the terms “cancer” and “mast cell related cancer” refer to a cancerous disease which characterized by the presence of too many MCs in the body for onset and/or progression. Cancerous MCs may be associated with phenotypes such uncontrolled proliferation, loss of specialized functions, immortality, significant metastatic potential, significant increase in antiapoptotic activity, rapid growth and proliferation rate, and certain characteristic morphology and cellular markers. In some circumstances, cancerous MCs will be in the form of a tumor, such cells may exist locally within an animal or circulate in the blood stream as independent cells. It will be appreciated that the term cancer as used herein encompasses all types of MC related cancers, at any stage and in any form. Non-limiting examples of MC related cancers can be any solid or non-solid MC cancer and/or cancer metastasis, including, but not limited to systemic mastocytosis (SM), mastocytoma, mast cell sarcoma (MCS) and mast cell activation Syndrome (MACS).

As used herein, the term “systemic mastocytosis (SM)” encompasses the five categories of SM defined by the World Health Organization according to their location and aggressiveness: cutaneous mastocytosis (CM), indolent SM (ISM), SM with associated clonal hematologic non-MC disease, aggressive SM (ASM), and MC leukemia (MCL). The prognosis of patients with ASM and MCL is poor due to an aggressive nature of the cells and their tendency to detach from the main tumor. Many of these tumors carry mutations in the tyrosine kinase receptor: c-Kit (stem cell factor receptor) that renders it constitutively activated leading to uncontrolled growth of the malignant MCs.

It should be further appreciated that the methods and compositions of the invention may be applicable for treating any of the allergic disorders disclosed above, for example asthma or dermatitis, as well as any associated conditions. It is understood that the interchangeably used terms “associated” and “related”, when referring to pathologies herein, mean diseases, disorders, conditions, or any pathologies which at least one of: share causalities, co-exist at a higher than coincidental frequency, or where at least one disease, disorder condition or pathology causes the second disease, disorder, condition or pathology.

It should be noted that in certain embodiments, the at least one mitochondrial targeting moiety used for the methods of the invention may be at least one of a lipohilic cation and/or at least one mitochondrial targeting sequence. In more specific embodiments, the at least one STAT3 inhibiting moiety used for the methods of the invention may be at least one of a small molecule based moiety, a nucleic acid based moiety, an amino acid based moiety and any combinations thereof.

In some embodiments, the at least one STAT3 inhibiting moiety used by the method of the invention may be any one of STAT3 Inhibitor V, 6-Nitrobenzo[b]thiophene 1,1-dioxide (Stattic); (1E,6E)-1,7-Bis(4-hydroxy-3-methoxyphenyl)-1,6-heptadiene-3,5-dione (curcumin); STAT3 Inhibitor XIII, C188-9; Protein Inhibitor Of Activated STAT, 3 (PIAS3), any derivatives, analogs or any combinations thereof or any vehicle, matrix, nano- or micro-particle comprising the same.

In some embodiments, the at least one STAT3 inhibiting moiety used by the method of the invention may be 6-Nitrobenzo[b]thiophene 1, 1-dioxide associated with TPP. It should be appreciated that such mitochondrial-targeted STAT3 inhibitor is referred to herein as MitoS. In other embodiments, the at least one STAT3 inhibiting moiety used by the method of the invention may be (1E,6E)-1,7-Bis(4-hydroxy-3-methoxyphenyl)-1,6-heptadiene-3,5-dione or derivatives thereof associated with TPP. In more specific embodiments, the methods of the invention may use (1E,6E)-1,7-Bis(4-hydroxy-3-methoxyphenyl)-1,6-heptadiene-3,5-dione or any derivative/s, salt/s or ester/s thereof associated with TPP. The association between (1E,6E)-1,7-Bis(4-hydroxy-3-methoxyphenyl)-1,6-heptadiene-3,5-dione may be with two TPP moieties. It should be noted that said compound is referred to herein and in the following Examples as mito-cur-1 (or Mitocur-1).

In some other embodiments, the methods of the invention may use at least one (1E,6E)-1,7-bis (4-hydroxyphenyl) hepta-1,6-diene-3,5-dione associated with TPP. The association between (1E,6E)-1,7-bis(4-hydroxyphenyl) hepta-1,6-diene-3,5-dione may be with two TPP moieties. It should be noted that said compound is referred to herein and in the following Examples as mito-cur-3 (or Mitocur-3).

In certain embodiments, the STAT3 inhibiting moiety may be encapsulated in a nano- or micro-particle. In other embodiments, the at least one STAT3 inhibiting moiety used by the method of the invention may be vehicle, matrix, nano- or micro-particle that comprise a STAT3 inhibiting moiety and at least one mitochondrial targeting moiety.

In some specific embodiments, the method of the invention may use liposomes encapsulating in their intraliposomal core at least one 6-Nitrobenzo[b]thiophene 1,1-dioxide and having at least one TPP associated onto the outer liposomal surface. Such compound is referred to herein as LipoS.

In further embodiments, the method of the invention may use at least one PIAS3 molecule or any fragments and peptides thereof that may be attached to at least one MTS that comprises Met Ser Val Leu Thr Pro Leu Leu Leu Arg Gly Leu Thr Gly Ser Ala Arg Arg Leu Pro Val Pro Arg Ala Lys Ile His Ser Leu, as denoted by SEQ ID NO. 8. In some specific embodiments said PIAS3-MTS may be referred to as PIAS3-mito, more specifically, said PIAS3-mito may comprise the amino acid sequence as denoted by SEQ ID NO. 16, or by SEQ ID NO. 18, or any variant or derivative thereof.

As noted above, the invention provides a method of treating, inhibiting, preventing or ameliorating MC activation related conditions.

The term “treatment” in accordance with disorders associated with activation of MC, specifically, allergic conditions may refer to one or more of the following: elimination of allergic reactions, reducing or decreasing the intensity or frequency of disorders associated with activation of MC, specifically, allergic conditions incidence. The treatment may be undertaken when a disorders associated with activation of MC, specifically, allergic conditions incidence is beginning or may be a continuous administration, for example by administration every 1 to 14 days, to prevent or decrease occurrence of allergic reaction in an individual prone to allergic reactions. Thus, the term “treatment” is also meant as prophylactic or ameliorating treatment.

The term “reduction” or “reduce”, as referred to herein, relates to a decrease in value, amount, or rate of the intensity or frequency of allergic reactions in the treated subject. A decrease or a reduction as referred to herein, relate to a reduction or lessening of a quantity or intensity of allergic reactions by any one of about 1% to 99.9%. More specifically, about 1% to 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%.

The term “inhibition” as referred to herein, relates to the retardation, retraining or reduction of the intensity or frequency of disorders associated with activation of MC, specifically, allergic conditions by about 1% to 99.9%. More specifically, by any one of about 1% to 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%.

The term “prophylaxis” refers to prevention or reduction the risk of occurrence of the biological or medical event, specifically, the occurrence or re occurrence of disorders associated with activation of MC, specifically, allergic conditions, that is sought to be prevented in a tissue, a system, animal or human by a researcher, veterinarian, medical doctor or other clinician, and the term “prophylactically effective amount” is intended to mean that amount of a pharmaceutical composition that will achieve this goal. Thus, in particular embodiments, the methods of the invention are particularly effective in the prophylaxis, i.e., prevention of conditions associated with activation of MC. Thus, subjects administered with said compositions are less likely to experience skin temperature rises or elevation in blood levels of mediators such as histamine, associated with allergic reactions, and said skin temperature rises or elevation in blood levels of mediators such as histamine are also less likely to re-occur in a subject who has already experienced them in the past.

The term “amelioration” as referred to herein, relates to a decrease in the deviation of the body temperature or reduced elevation in blood levels of mediators such as histamine of a subject suffering from allergic condition, from the normal physiological temperature range for that subject or normal level of said mediators. More specifically, “amelioration” relates to an improvement in a subject's allergic reaction intensity. For instance, as a non-limiting example, the symptoms of a subject suffering from disorders associated with activation of MC, specifically allergic conditions, are ameliorated by the blood histamine levels or body-temperature reducing treatment with the composition of the invention or according to the method of the invention.

The method of the invention involves administration of therapeutically effective amount of the mitochondrial-targeted STATS inhibitor/s of the invention or specifically, stattic-TPP, Lipo-stattic, Curcumin-TPP or PIAS3-mito. The term “effective amount” as used herein is that determined by such considerations as are known to the man of skill in the art. The amount must be sufficient to prevent or ameliorate tissue damage caused by the MC activation related disorders treated, specifically, any one of allergic reaction, asthma, eczema, dermatitis, allergic rhinitis allergic conjunctivitis, anaphylaxis, mastocytosis and mast cell tumors. Dosing is dependent on the severity of the symptoms and on the responsiveness of the subject to the active drug. Medically trained professionals can easily determine the optimum dosage, dosing methodology and repetition rates. In any case, the attending physician, taking into consideration the age, sex, weight and state of the disease of the subject to be treated, will determine the dose.

More specifically, the compositions containing the mitochondrial-targeted STAT3 inhibitor/s of the present invention, or any combination, mixture or cocktail thereof can be administered for prophylactic and/or therapeutic treatments. In therapeutic application, compositions are administered to a patient already affected by disorder associated with activation of MC, specifically, allergic conditions (e.g., allergic reaction, asthma, eczema, dermatitis, allergic rhinitis allergic conjunctivitis, anaphylaxis, mastocytosis and mast cell tumors) in an amount sufficient to cure or at least partially arrest the condition and its complications. An amount adequate to accomplish this is defined as a “therapeutically effective dose.” Amounts effective for this use will depend upon the severity of the condition and the general state of the patient's own immune system, but generally range from about 0.01 to about 100 mg/Kg, specifically, about 0.01 to about 1000, 900, 800, 700, 600, 500, 400, 300, 200 and 100 mg, more specifically, about 250-450 mg of the mitochondrial-targeted STAT3 inhibitor/s of the invention per dose, or with dosages of from 0.1 to 500 mg/kg, more specifically, 500, 400, 300, 200, 100, 95, 90, 85, 80, 75, 70, 65, 60, 55, 50, 45, 40, 35, 30, 25, 20, 15 and 10, 9, 8, 7, 6, 5, 4, 3, 2, 1 mg per Kg of body weight. Specifically, about 0.01 to about 100 mg per Kg of body weight being more commonly used. Single or multiple administrations on a daily, weekly or monthly schedule can be carried out with dose levels and pattern being selected by the treating physician. More specific embodiments relate to the use of typically 2-3 doses per week, containing 40-80 mg per Kg body weight, but not more than a daily dose of 500 mg/Kg body weight. The invention further provides a method for preventing or reducing the risk of developing disorders associated with activation of MC, specifically, allergic conditions such as allergic-skin disorders and allergic respiratory disorders, specifically, asthma, eczema, dermatitis, allergic rhinitis allergic conjunctivitis, anaphylaxis, mastocytosis and mast cell tumors. Such method comprises the administration of a prophylactically effective amount of the mitochondrial-targeted STAT3 inhibitor/s, or combination of more than one mitochondrial-targeted STAT3 inhibitor/s according to the invention, or pharmaceutical compositions thereof, to a person at risk of developing an allergic-related disorder such as allergic-skin disorders, allergic-respiratory disorders and associated disorders, specifically, asthma, eczema, dermatitis, allergic rhinitis allergic conjunctivitis, anaphylaxis, mastocytosis and mast cell tumors.

The term “prophylactically effective amount” is intended to mean that amount of a pharmaceutical combined composition that will prevent or reduce the risk of occurrence or recurrence of the biological or medical event that is sought to be prevented in a tissue, a system, animal or human by a researcher, veterinarian, medical doctor or other clinician. In prophylactic applications, compositions containing the mitochondrial-targeted STAT3 inhibitor/s of the invention or any combination, mixture or cocktail thereof are administered to a patient who is at risk of developing the disease state to enhance the patient's resistance. Such an amount is defined to be a “prophylactically effective dose”. In this use, the precise amounts again depend upon the patient's state of health and general level of immunity, but generally range from 0.1 to 1000 mg per dose, specifically, 900, 800, 700, 600, 500, 400, 300, 200 and 100 mg, more specifically, about 250-450 mg of the mitochondrial-targeted STAT3 inhibitor/s of the invention per dose, or with dosages of from 0.1 to 500 mg/kg, more specifically, 500, 400, 300, 200, 100, 95, 90, 85, 80, 75, 70, 65, 60, 55, 50, 45, 40, 35, 30, 25, 20, 15 and 10, 9, 8, 7, 6, 5, 4, 3, 2, 1 mg per Kg of body weight, especially 0.1 to 100 mg per Kg of body weight per dose, specifically, 10, 20, 30, 40, 50, 60, 70, 80, 90 and 100 mg per Kg of body weight per dose.

Additionally, the administration of the mitochondrial-targeted STAT3 inhibitor/s of the invention, or pharmaceutical compositions thereof, according to the invention, may be periodic, for example, the periodic administration may be effected twice daily, three time daily, or at least one daily for at least about three days to three months. The advantages of lower doses are evident to those of skill in the art. These include, inter alia, a lower risk of side effects, especially in long-term use, and a lower risk of the patients becoming desensitized to the treatment.

In another embodiment, treatment using the mitochondrial-targeted STAT3 inhibitor/s of the invention, or pharmaceutical compositions thereof, may be effected following at least 1, 2, 3, 4, 5, 6, 7, 8, 9, 10, 14, 30, 60, 90 days of treatment, and proceeding on to treatment for life.

It should be noted that the treatment of different conditions may indicate the use of different doses or different time periods; these will be evident to the skilled medical practitioner.

As used herein, “disease”, “disorder”, “condition” and the like, as they relate to a subject's health, are used interchangeably and have meanings ascribed to each and all of such terms.

The present invention relates to the treatment of subjects, or patients, in need thereof. By “patient” or “subject in need” it is meant any organism who may be affected by the above-mentioned conditions, and to whom the treatment and diagnosis methods herein described is desired, including humans, domestic and non-domestic mammals such as canine and feline subjects, bovine, simian, equine and murine subjects, rodents, domestic birds, aquaculture, fish and exotic aquarium fish. It should be appreciated that the treated subject may be also any reptile or zoo animal. More specifically, the composition/s and method/s of the invention are intended for mammals. By “mammalian subject” is meant any mammal for which the proposed therapy is desired, including human, equine, canine, and feline subjects, most specifically humans. It should be noted that specifically in cases of non-human subjects, the method of the invention may be performed using administration via injection, drinking water, feed, spraying, oral gavage and directly into the digestive tract of subjects in need thereof. It should be noted that administering of the mitochondrial-targeted STAT3 inhibitor/s, specifically, stattic-TPP (MitoS), Lipo-stattic (LipoS), Curcumin-TPP (Mitocur-1, and Mitocur-3), or any salt, base, ester or amide thereof or PIAS3-MTS, according to the invention to the patient includes both self-administration and administration to the patient by another person. Specifically, said subject is a human subject suffering from an allergic condition.

It is understood that the method of the invention involves administration by any one of nasal, transdermal, pulmonary, oral, buccal or sublingual administration, or any combinations thereof, specifically a combination of nasal and oral routes. However, it should be appreciated that the mitochondrial-targeted STAT3 inhibitor/s used by the method of the invention, specifically, stattic-TPP, Lipo-stattic, Curcumin-TPP, may be administered by injection (subcutaneously, intraperitoneally, intramuscularly, intravenously), rectally, vaginally, intraocular, sprayed at armpit and any combination thereof.

In many instances, therapies employing two or more administration methods are required to adequately address different medical conditions and/or effects of a certain disorder under treatment. Thus, at least one mitochondrial-targeted STAT3 inhibitor/s or specifically, stattic-TPP, Lipo-stattic, Curcumin-TPP, or any salts, esters or base thereof or any mixture thereof, may be administered by the method of the invention using a combination of at least two administration methods. Combining these at least two administration methods safely and effectively improves overall beneficial effect on the disorders addressed by this invention.

Thus, it is understood that according to some embodiments of the method of the invention, the method further comprises an oral administration of mitochondrial-targeted STAT3 inhibitor/s or specifically, stattic-TPP, Lipo-stattic, Curcumin-TPP, before, simultaneously with, after or any combination thereof, the intranasal or pulmonary administration of the mitochondrial-targeted STAT3 inhibitor/s.

More specifically, the term “administration” when relating to treatment of respiratory disorders is preferably pulmonary delivery by oral inhalation, such as by using liquid nebulizers, aerosol-based metered dose inhalers (MDIs), or dry powder dispersion devices, or by intraperitoneal injection. Alternatively, the administration may be any one of sublingual, buccal, parenteral, intravenous, intramuscular, subcutaneous, intramedullary, or transdermal.

Specifically, asthma may be treated by pulmonary administration of the mitochondrial-targeted STATS inhibitor/s of the invention, e.g., by inhalation or insufflation of powders or aerosols, including by nebulizer; intratracheal, intranasal, epidermal and transdermal.

It should be noted that for the treatment of respiratory disorders, preferably asthma, pre-conditioning (initiating treatment shortly before potential exposure) may provide further benefit.

As indicated above, in addition to the intraperitoneal, intranasal and transdermal routes, the compositions used in the uses, methods and kits of the invention may be adapted for administration by any other appropriate route, for example by the parenteral, oral (including buccal or sublingual), rectal, topical (including buccal or sublingual) or vaginal route. Such formulations may be prepared by any method known in the art of pharmacy, for example by bringing into association the active ingredient with the carrier(s) or excipient(s).

Pharmaceutical formulations adapted for rectal administration may be presented as suppositories or enemas.

Pharmaceutical formulations adapted for vaginal administration may be presented as pessaries, tampons, creams, gels, pastes, foams or spray formulations.

Compositions and formulations for oral administration include powders or granules, suspensions or solutions in water or non-aqueous media, capsules, sachets, lozenges (including liquid-filled), chews, multi- and nano-particulates, gels, solid solution, liposome, films, ovules, sprays or tablets. Thickeners, flavoring agents, diluents, emulsifiers, dispersing aids or binders may be desirable.

Pharmaceutical compositions used to treat subjects in need thereof according to the invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general formulations are prepared by uniformly and intimately bringing into association the active ingredients with liquid carriers or finely divided solid carriers or both, and then, if necessary, shaping the product. The compositions may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers. The pharmaceutical compositions of the present invention also include, but are not limited to, emulsions and liposome-containing formulations.

It should be understood that in addition to the ingredients particularly mentioned above, the formulations may also include other agents conventional in the art having regard to the type of formulation in question, for example those suitable for oral administration may include flavoring agents.

The compounds of the invention may also be administered directly to the eye or ear, typically in the form of drops of a micronised suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose or methyl cellulose or a heteropolysaccharide polymer, for example, gelan gum, may be incorporated together with a preservative, such as benzalkonium chloride. Such formulations may also be delivered by iontophoresis.

Formulations for ocular and aural administration may be formulated to be immediate and/or modified release. Modified release includes delayed, sustained, pulsed, controlled, targeted, and programmed release.

Preferred unit dosage formulations are those containing a daily dose or sub-dose, as herein above recited, or an appropriate fraction thereof, of an active ingredient.

Disclosed and described, it is to be understood that this invention is not limited to the particular examples, process steps, and materials disclosed herein as such process steps and materials may vary somewhat. It is also to be understood that the terminology used herein is used for the purpose of describing particular embodiments only and not intended to be limiting since the scope of the present invention will be limited only by the appended claims and equivalents thereof.

Throughout this application various publications are referred to in parentheses. All of these publications, and publications referred to therein, are fully incorporated herein by reference. The list of references is given at the end of the description, immediately preceding the claims.

It must be noted that, as used in this specification and the appended claims, the singular forms “a”, “an” and “the” include plural referents unless the content clearly dictates otherwise.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The following Examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

### Experimental Procedures

### Mice

C3H 6-7 week male and female WT mice were purchased from Jackson Lab (Cambridge, Mass., USA), mice were fed a regular diet and with drinking water ad libitum. Experiments were approved by The Hebrew University Ethical Committee for Animal Experimentation.

Antibodies

The antibody anti-PIAS3 was purchased from Sigma-Aldrich Corp. (St. Louis, Mo., USA). Anti-STAT3, anti-phospho-STAT3 serine 727, anti-phospho-ERK and anti-cytochrome c antibodies were purchased from Santa Cruz Biotechnology (Santa Cruz, Calif.). Anti-caspase3, anti-ERK1/2 anti-pyruvate dehydrogenase al subunit were purchased from Cell Signaling Technology (Beverly, Mass., USA).

Chemical Inhibitor Treatment

Stattic, and U0126 were purchased from Sigma-Aldrich Corp. (St. Louis, Mo., USA) 5,15-DPP was purchased from Merck-Millipore (Billerica, Ma, USA). All inhibitors were dissolved in 100% DMSO (Sigma-Aldrich Corp.) according to the manufacturer's instructions.

Cell Culture

RBL-2H3 cells were maintained in RPMI 1640 medium as previously described1. Bone marrow was isolated from 5-6 week-old mice, and BMMC were cultured in RPMI medium supplemented with 20 ng/ml interleukin-3 (IL-3) and 20 ng/ml SCF as previously described2, 3. Cells were generally grown for a minimum of 4 weeks and used when >90% of the population expressed FcεRI. BMMC and RBL cells were sensitized first with anti-DNP IgE monoclonal antibody (SPE-7; Sigma-Aldrich Corp.) and then challenged with DNP-BSA (Sigma-Aldrich Corp.). IgE antibody was centrifuged (18,000 g, 5 min) before use to remove aggregates. Cell activation was verified by demonstrating an increased phosphorylation of extracellular signal-regulated kinase (ERK) following the Ag challenge. For glycolysis elimination cells were maintained in glucose-free RPMI supplemented with 10% dialyzed fetal calf serum and 5 mM galactose.

Cord Blood Derived Mast Cells' Culture and Activation

CBMC were prepared from umbilical cord blood (CB) of healthy donors according to a well described method4. Briefly, fresh CB diluted in Hank's solution was loaded on Ficoll-Hypaque (Amersham Pharmacia Biotech AB, Uppsala, Sweden) and centrifuged (350 g, 25 min) Mononuclear cells were washed twice and resuspended in MEM-α medium supplemented with heat-inactivated fetal bovine serum 10%, penicillin 100 U/ml, streptomycin 100 μg/ml, and ribonucleosides 10 μg/ml. All media and supplements were purchased from Biological Industries, Beit Haemek, Israel. CBMC growth media also contained recombinant human stem cell factor (SCF; 100 ng/ml; Peprotech, Rocky Hill, N.J.), human IL-6 (10 ng/ml; Peprotech), and PGE2 (0.3 μM; Sigma-Aldrich). Cells were maintained in a humidified incubator (37° C., 5% CO2) with media replaced on a weekly basis. Following a 6-8 week culture period, the CBMCs were examined for maturity by acidic toluidine blue staining (positive>90%), and for viability by Trypan blue exclusion (negative>95%), and used thereafter.

For activation experiments CBMC were sensitized by addition of human myeloma IgE (0.3 μg/ml; Calbiochem-Merck, Schwalbach, Germany) and human IL-4 (0.10 ng/ml; Peprotech) to cell cultures for 4-5 days in a humidified incubator (37° C., 5% CO2). CBMC, washed and resuspended in TG++ buffer (1×105/100 μl) were activated by incubation with rabbit α-human-IgE (5 μg/ml; Dako) for 30 min at 37° C. In inhibition experiments, 60 μM Stattic or DMSO alone (control) were added for 5 min, cells were centrifuged, resuspended in new TG++ buffer and activated as above. Supernatants and cell pellets were collected for assessment of β-hexosaminidase release, as described below.

Mitochondrial Isolation

RBL cells were washed once in cold PBS prior to homogenization. 9×106 cells were homogenized in buffer A (320 mm sucrose, 5 mm Tris, 2 mm EGTA, pH 7.4) with 10 strokes of a Teflon Dounce homogenizer. The procedure was performed at 4° C. The homogenate was centrifuged for 3 min at 2000 g and the supernatant kept. This was repeated twice, and the supernatants combined then centrifuged for 3 min at 2000 g. to remove the nuclei and cell debris. The supernatant was transferred to a clean tube and centrifuged for 10 min at 12,000 g to pellet mitochondria, washed twice resuspended in a small volume of buffer A and stored at −70° C. In order to prepare mitoplasts, the mitochondrial pellet was resuspended in buffer A with the addition of 0.02% (w/v) digitonin. The pellet was resuspended and washed twice in large volumes of digitonin-free buffer A.

Measurements of Enzymatic Activity

The enzymatic activities of respiratory chain complexes were measured at 37° C. by standard spectrophotometric methods as previously described5, 6. Briefly, complex II was measured as succinate dehydrogenase (SDH) based on the succinate-mediated phenazine methosulfate reduction of dichloroindophenol at 600 nm Complex 2+3 were measured as succinate cytochrome c reductase and after the reduction of oxidized cytochrome c at 550 nm. Complex 4 (cytochrome c oxidase) was measured by following the oxidation of reduced cytochrome c at 550 nm.

Citrate synthase (CS), an ubiquitous mitochondrial matrix enzyme, serving as a control, was measured in the presence of acetylCoA and oxaloacetate by monitoring the liberation of CoASH coupled to 5′,5′-dithiobis(2-nitrobenzoic) acid at 412 nm. Activities are presented as a ratio to CS. Protein concentration was determined by the Lowry method and calculated according to a bovine serum albumin (BSA) standard curve.

Expression Constructs

Mouse PIAS3 was constructed with a mitochondrial target sequence by cloning into the SalI and NotI sites of the pCMV/myc/mito vector (Invitrogen, Carlsbad Ca, USA).

siRNA

PIAS3 siGENOME SMARTpool siRNA oligomers for rat were purchased from Dharmacon (Thermo Fischer, Waltham, Mass.).

PIAS3 TriFECTa® RNAi Kit for mouse was purchased from IDT (Integrated DNA Technologies, Jerusalem, Israel).

β-Hexosaminidase Release Assay (Degranulation Assay)

1×104 RBL or 3×104 BMMC were immunologically activated as described above. After IgE incubation, the cells were washed three times with 5 mL of degranulation buffer (NaCl 130 mM, KCl 5 mM, Glucose 5.6 mM, MgCl2 1 mM, CaCl2 1.2 mM, Hepes pH 7.4 10 mM, BSA 0.1%) and resuspended in 200 μL of the same buffer with DNP-albumin for 30 min. The release of β-hexosaminidase was determined in triplicates in a 96-well plate. Aliquots (20 μL) of supernatants and cell lysates were incubated for 30 minutes with 100 μL of substrate solution (1.3 mg/mL p-nitrophenyl-b-D-2-acetamido-2-deoxyglucopyranozide in 0.1 M citrate [pH 4.51]). The reaction was stopped by the addition of 200 μL of 0.2 M glycine (pH 10.7). The plate OD was read in an enzyme-linked immunosorbent assay (ELISA) reader at a wavelength of 405 nm Percent release values for each experimental condition were calculated3.

ATP Determination

ATP levels were measured using the ATPLite luminescence-based assay according to the manufacturer's instructions (Perkin Elmer, Waltham, Mass., USA).

Oxygen Consumption

Oxygen consumption rate (OCR) was measured using an XF24 extracellular flux analyzer (Seahorse Biosciences, North Billeric, Mass., USA).

Gel Electrophoresis and Western Blots

RBL cells and mitochondrial pellets were lysed by the addition of lysis buffer (50 mM Tris-HCl pH 7.4, 1% Nonidet P-40, 0.25% Na-deoxycholate, 150 mM NaCl, 1 mM EDTA, 1 mM PMSF) supplemented with 15 μl protease inhibitor (Sigma-Aldrich). Cells and mitochondria were vortexed and incubated on ice for 15 min. The lysates were then centrifuged at 14,000 g for 15 min and pellets were discarded. The protein concentration of each sample was determined using Bradford Reagent (Sigma-Aldrich). Proteins were resolved by 10-15% SDS-PAGE under reducing conditions and transferred to polyvinylidene difluoride membranes (Millipore Billerica, Ma). The membranes were incubated in 5% fat-free milk and TBST (10 mM Tris-HCl, pH 7.4, 150 mM NaCl, 0.1% Tween 20) for 1 h and then in 5% BSA and TBST containing various dilutions of primary Abs for 18 h at 4° C. The membranes were washed three times with TBST for 5 mM before and after each incubation with secondary Ab. The proteins were detected with the appropriate secondary Ab (1 h; room temperature) coupled to HRP-conjugated goat anti-rabbit or anti-mouse Ab and visualized by chemiluminescence according to the manufacturer's instructions (Pierce).

Transfection

Amaxa Nucleofector technology (Amaxa, Cologne, Germany) was used to transfect cells. A total of 2×106 RBL or 1.5×106 BMMCs were transfected with 3 μg of the selected siRNA oligonucleotide or scrambled nonrelevant siRNA according to the manufacturer's protocol. For plasmid transfection 2 μg was used. Briefly, the cells were resuspended in 100 μl Ingenio solution (Mirus, Wis., USA), either DNA (plasmid) or RNA was added, and the mixture was transferred to an electroporation cuvette. Electroporation was performed using the T-20 program for RBL and T-17 for BMMC3, 7.

ImageStream Flow Cytometry

For ImageStream analysis (Amnis Corp, Seattle, Wash.), RBL cells were incubated with anti-DNP IgE monoclonal antibody and 1.5 μM MitoTracker Red CM-H2XRos (Molecular Probes, Eugene, Oreg. USA) for 90 min. Cells were washed three times in PBS and then challenged with DNP-BSA for the indicated times. Cells were washed three times in PBS, fixed in 10% paraformaldehyde, permeabilized with PBS 0.1% triton, and blocked in PBS 0.5% BSA. Anti-PIAS3 antibody and goat anti-rabbit IgG H&L (DyLight® 650; Abcam, Cambridge, Mass.) were used to stain PIAS3. Hoechst 33258 (Sigma-Aldrich) was used to stain nuclei. Fluorescence imaging was carried out with the ImageStream flow cytometer (Amnis Corp). Data analysis was performed with IDEAS software version 1.10, with compensation according to the software standards.

An ImageStream instrument automatically acquires up to six different spatially registered images (bright-field, dark-field, and four fluorescent images) per cell at rates on the order of thousands of objects per minute, using a digital charge-coupled device (CCD) camera. The digital imagery obtained is analyzed using the IDEAS statistical image analysis program, which provides tools for the objective numerical scoring and discrimination of cells based on the characteristics of their imagery. The ability to numerically score large numbers of automatically acquired images is ideally suited to the analysis of mitochondrial translocation within primary immune system cells.

Measurements of Secreted Mediators

1×106 BMMC were immunologically activated as described above. After IgE incubation, the cells were washed three times in PBS and incubated with DNP-albumin (Sigma-Aldrich) for 30 mM in complete growth medium. Supernatant was collected and Granzyme B and TNFα content were measured using ELISA Kits (eBioscience).

In Vivo Histamine Release Assay

WT C3H mice were sensitized with 3 μg murine IgE anti-DNP mAb SPE-7 by means of intravenous injection in the tail vein. Twenty-four hours later, mice were treated with 40 mg/kg of static dissolved in corn oil or corn oil alone for 2.5 hours, then mice were challenged with i.v. injection of DNP-BSA (250 μg per mouse). 100 μl of blood was collected immediately and added to an equal volume of pre-cold Alsever's Solution. Blood was centrifuged 2000 g for 20 mM and plasma supernatant was collected. Plasma histamine levels were determined by using competitive ELISA (IBL International Gmbh, Hamburg, Germany).

### Synthesis of curcumine-based mitochondrial targeted STAT inhibitors

### Mitocurcuminoid-1

### 4-(3-Chloropropoxy)-3-methoxybenzaldehyde

A mixture of vanillin (5 g, 30.2 mM), 1-bromo-3-chloropropane (10.36 ml, 61.1 mM), potassium carbonate (18.1 g, 122.6 mM) and PEG-400 (15 ml) was stirred at room temperature for 3 h. The mixture was diluted with crushed ice and acidified with diluted HCl. After stirring for 15 mM, hexane (100 ml) was added to the solution and stirred for 30 min. The precipitated solid was filtered, washed with hexane and dried (5.1 g, 68%), mp 46-48° C. ¹H NMR (400 MHz, CDCl₃): δ 9.86 (1H, s), 7.45 (1H, d, J=8.4, 2.0 Hz), 7.42 (1H, d, J=2.0 Hz), 7.01 (1H, d, J=8.4 Hz), 4.26 (2H, t, J=6.0 Hz), 3.92 (3H, s), 3.78 (2H, t, J=6.2 Hz), 2.33 (2H, quintet, J=6.1 Hz).

### (1E,6E)-1,7-Bis[4-(3-chloropropoxy)-3-methoxyphenyl]hepta-1,6-diene-3,5-dione

To a solution of boric oxide (1.05 g, 15.22 mM) in DMF (4 ml) was added acetyl acetone (1.35 ml, 13.13 mM) followed by tributyl borate (7.16 ml, 26.52 mM) at 65° C. and stirred for 15 min. To the above borate complex, 4(3-chloropropoxy)-3-methoxybenzaldehyde (6 g, 26.25 mM) was added and stirred for 5 min. A mixture of n-butylamine (0.25 ml) and acetic acid (0.75 ml) in DMF (4 ml) was added to the reaction mixture and heated to 95° C. for 4 h. After cooling to 15° C., acetic acid (20%, 150 ml) was added with stirring and again the reaction mixture was stirred at 70° C. for another 1 h. Then it was cooled to room temperature and extracted with chloroform. The combined chloroform layer was washed with brine and dried over sodium sulfate. The solution was filtered and evaporated the solvent. The residue was chromatographed over silica gel column using hexane-ethyl acetate (80:20) as eluent to give the product as yellow color solid (3.5 g, 26%), mp 132-134° C.

### 1,7-Bis{3-methoxy-4-[3-(triphenylphosphonium)propoxy]-phenyl}hepta-1,6-diene-3,5-dione dichloride

A mixture of 1,7-Bis[4-(3-chloropropoxy)-3-methoxyphenyl]hepta-1,6-diene-3,5-dione (350 mg, 0.67 mM), triphenyl phosphine (0.7 g, 2.68 mM), n-butanol (5 ml) and catalytic amount of potassium iodide was stirred at 100° C. for 4 h. The reaction mixture was attained to room temperature and the solution was slowly added with stirring to diethyl ether (75 ml) for 10 mM Few drops of chloroform was added to dissolve the residue in the R.B. and added this solution to the ether. After the addition, the solution was stirred for 30 min and filtered, washed with ether and dried to give the product. The crude product was chromatographed over silica gel column using chloroform-methanol (90:10) as eluents to give the product, which was dissolved in minimum amount of chloroform and slowly added to diethyl ether with stirring. The precipitated product was filtered, washed with ether and dried to give the product as an yellow color solid (450 mg, 65%), mp 130-140° C. ¹H NMR (400 MHz, DMSO-d₆): δ 2.04 (4H, m), 3.74 (4H, m), 3.86 (6H, s), 4.19 (4H, m), 6.19 (1H, s), 6.89 (2H, d, J=15.6 Hz), 7.01 (2H, d, J=8.0 Hz), 7.27 (2H, d, J=8.0 Hz), 7.39 (2H, br s), 7.61 (2H, d, J=15.6 Hz), 7.79-7.94 (30H, m); ¹³C NMR (100 MHz, DMSO-d₆): δ 183.2, 149.6, 149.4, 140.2, 134.9 (d, J=3 Hz), 133.6 (d, J=10 Hz), 130.3 (d, J=12 Hz), 128.3, 122.6, 122.5, 118.3 (d, J=86 Hz), 113.5, 111.1, 101.0, 67.7 (d, J=17 Hz), 55.9, 22.2, 17.8 (d, J=53 Hz); HPLC: 96.5%.

### Mitocurcuminoid-3

### 1,7-Bis[4-(3-chloropropoxy)phenyl]hepta-1,6-diene-3,5-dione

To a solution of boric oxide (2.01 g, 29.23 mM) in DMF (5.3 ml) was added acetyl acetone (2.5 ml, 25.1 mM) followed by tributyl borate (13.4 ml, 50.9 mM) at 65° C. and stirred for 15 min. To the above borate complex, 4(3-chloropropoxy)benzaldehyde (10 g, 50.4 mM) was added and stirred for 5 min. A mixture of n-butylamine (0.44 ml) and acetic acid (1.45 ml) in DMF (5.3 ml) was added to the reaction mixture and heated to 95° C. for 4 h. After cooling to 15° C., acetic acid (20%, 350 ml) was added with stirring and again the reaction mixture was stirred at 70° C. for another 1 h. Then it was cooled to 5-10° C., filtered the solids, washed with ice cold water and dried. The residue was chromatographed over silica gel column using hexane-ethyl acetate (80:20) as eluent to give the product as yellow color solid (7.5 g, 32%).

### 1,7-{4-[3-(triphenylphosphonium)propoxy]phenyl}hepta-1,6-diene-3,5-dione dichloride

A mixture of 1,7-Bis[4-(3-chloropropoxy)phenyl]hepta-1,6-diene-3,5-dione (3.0 g, 6.5 mM), triphenyl phosphine (6.81 g, 26 mM), n-butanol (25 ml) and catalytic amount of potassium iodide was stirred at 100° C. for 8 h. The reaction mixture was attained to room temperature and the solution was slowly added with stirring to diethyl ether (75 ml) for 10 min. Few drops of chloroform was added to dissolve the residue in the R.B. and added this solution to the ether. After the addition, the solution was stirred for 30 min and filtered, washed with ether and dried to give the product. The crude product was chromatographed over silica gel column using chloroform-methanol (90:10) as eluents to give the product, which was dissolved in minimum amount of chloroform and slowly added to diethyl ether with stirring. This process was repeated for 4 times. The precipitated product was filtered, washed with ether and dried to give the product as an yellow color solid (2.2 g, 34%), mp 145-160° C. ¹H NMR (400 MHz, DMSO-d₆): δ 2.03 (4H, m), 3.78 (4H, m), 4.21 (4H, m), 6.17 (1H, s), 6.82 (2H, d, J=15.6 Hz), 7.02 (4H, d, J=8.4 Hz), 7.62 (2H, d, J=15.6 Hz), 7.70 (4H, d, J=8.0 Hz), 7.79-7.92 (30H, m); ¹³C NMR (100 MHz, DMSO-d₆): δ 183.2, 159.8, 139.9, 134.9 (d, J=3 Hz), 133.6 (d, J=10 Hz), 130.3 (d, J=13 Hz), 130.1, 127.7, 122.1, 118.3 (d, J=86 Hz), 115.1, 101.2, 66.9 (d, J=17 Hz), 22.1, 17.9 (d, J=52 Hz); HPLC: 94.4%.

Synthesis of TPP Conjugated to Stattic

The first step involved a SN2 reaction of alkyl halides and thiols, for example reacting 4-bromo-benzenethiol and 2-bromo-1,1-diethoxy-ethane to form 1, bromo-4-(2,2-diethoxy-ethylsulfanyl)-benzene. The second step included polyphosphoric acid (PPA) for ring closure to form a brome substituted benzo[b]thiophene such as 5-bromo-benzo[b]thiophene.

In a successive steps, the TPP is conjugated via a reaction with phosphine such as triphenylphosphine to form a phosphonium salt. The benzo[b]thiophene moiety is transformed into benzo[b]thiophene, 1,1-dioxde.

Statistical Analysis

Either the one sample t test or the 2-tailed Student t test was performed when appropriate. The exact Mann Whitney one tail test was used for in vivo studies. Data are reported as means±SEMs.

### Example 1

### Mitochondrial ATP and Mast Cell Exocytosis

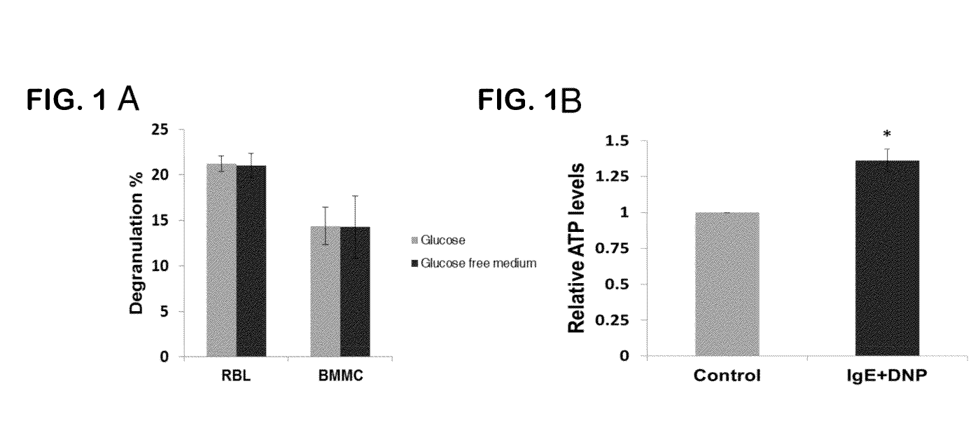

In order to determine whether mitochondrial ATP produced by OXPHOS is involved in mast cell degranulation, glycolysis was prevented and β-hexosaminidase release was determined Rat basophil leukemia (RBL) cells and bone marrow derived mast cells (BMMC) were cultured for 24 h in a glucose-free, OXPHOS dependent medium supplemented with dialyzed serum as an energy source or in complete medium used as control. These cells were activated by 2 h incubation with IgE followed by 30 min incubation with DNP-BSA. As shown in FIG. 1A, no significant change in degranulation was observed between these two groups indicating that the major part of the energy for degranulation is derived from mitochondrial ATP. The effect of immunological activation on mitochondrial ATP levels was determined in immunologically activated RBL cells grown in glucose-free medium (FIG. 1B). The ATP content was determined after 5 min incubation with DNP-BSA in order to determine its role in the initiation of the exocytosis process Immunological trigger caused a significant increase of nearly 40% in the level of ATP in the activated cells.

### Example 2

### Effect of STAT3 Inhibition on Mitochondrial ATP Production and Oxygen Consumption

STAT3 and its serine 727 phosphorylated form were previously reported to regulate mitochondrial ATP production in a variety of cells.¹²⁻¹⁴ Thus, next we determined the role if any played by mitochondrial STAT3 in mast cell function.

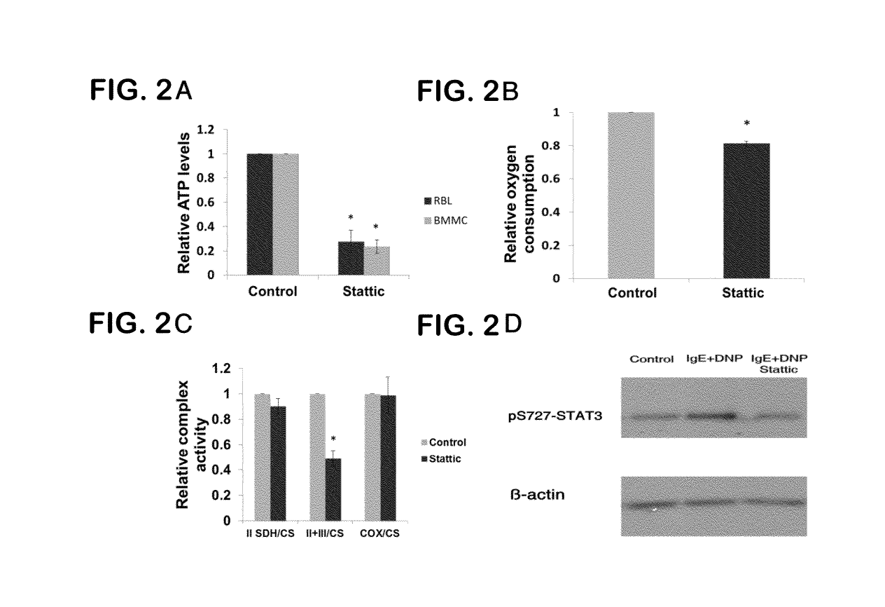

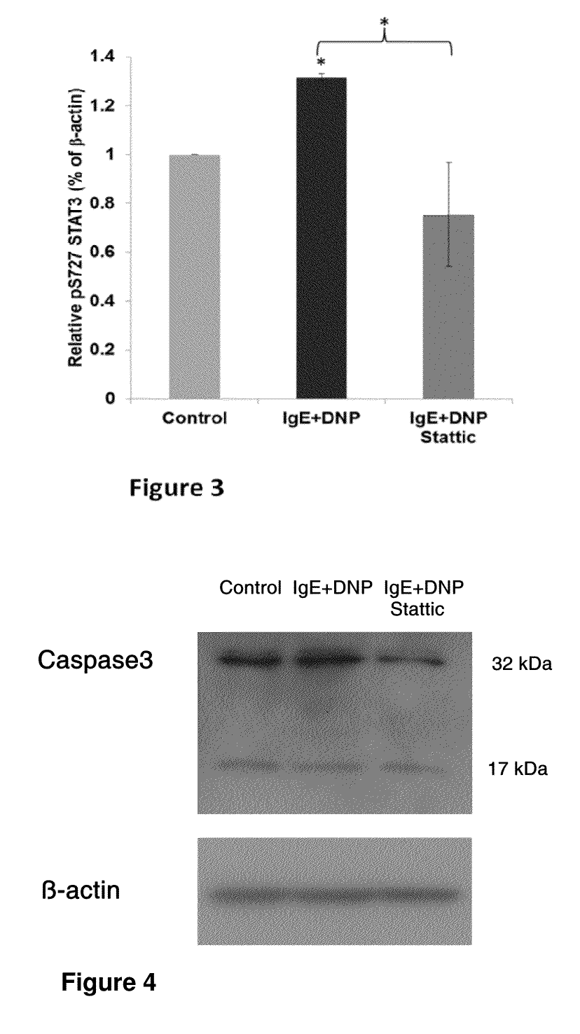

In order to inhibit STAT3 and distinguish between its effect on transcription and that on the modulation of mitochondrial activities, RBL and BMMC were incubated with the STAT3 inhibitor, Stattic 24 for 20 min (60 μM dissolved in 100% DMSO), a time frame which should not allow protein synthesis of STAT3 target genes. Control cells were treated with the same volume of DMSO alone. The effects of STAT3 inhibition on ATP levels, oxygen consumption and Electron Transfer Chain (ETC) activity (as indicators of OXPHOS activity) were determined. As shown in FIG. 2A-C, STAT3 inhibition resulted in a significant reduction in ATP levels (72.0±9.0%; p<0.05), oxygen consumption (18.6±8.3%; p<0.05) and complex 2+3 activity (50.8±6.1%, p<0.05) in RBL cells. No significant change in cytochrome c oxidase (COX, complex 4) and succinate dehydrogenase (SDH, complex 2) activities were observed (FIG. 2C). These results indicate a selective effect of STAT3 inhibition on cytochrome c reductase (complex 3) activity. Furthermore, as shown in FIG. 2A, a similar reduction in ATP levels (76.4±9.3%; p<0.05) was observed in BMMC treated with Stattic. Next it was determined whether these effects correlated with the inhibition of STAT3 serine 727 phosphorylation. The level of serine 727 phosphorylation in total cell lysate was determined in RBL cells treated with Stattic for 20 min and immunologically activated for 5 min. As shown in FIG. 2D a significant reduction in the phosphorylation levels of STAT3 was observed in these activated cells treated with Stattic. Densitometry quantification of this Western blot is shown in FIG. 3. As reduction in ATP content is also a marker for apoptosis, the inventors next determined whether the inhibitory effect of Stattic initiated apoptosis by determining the levels of cleaved caspase3. As shown in FIG. 4, no change in caspase3 levels was observed in cells treated with Stattic.

### Example 3

### The Role Played by STAT3 in Mast Cell Function

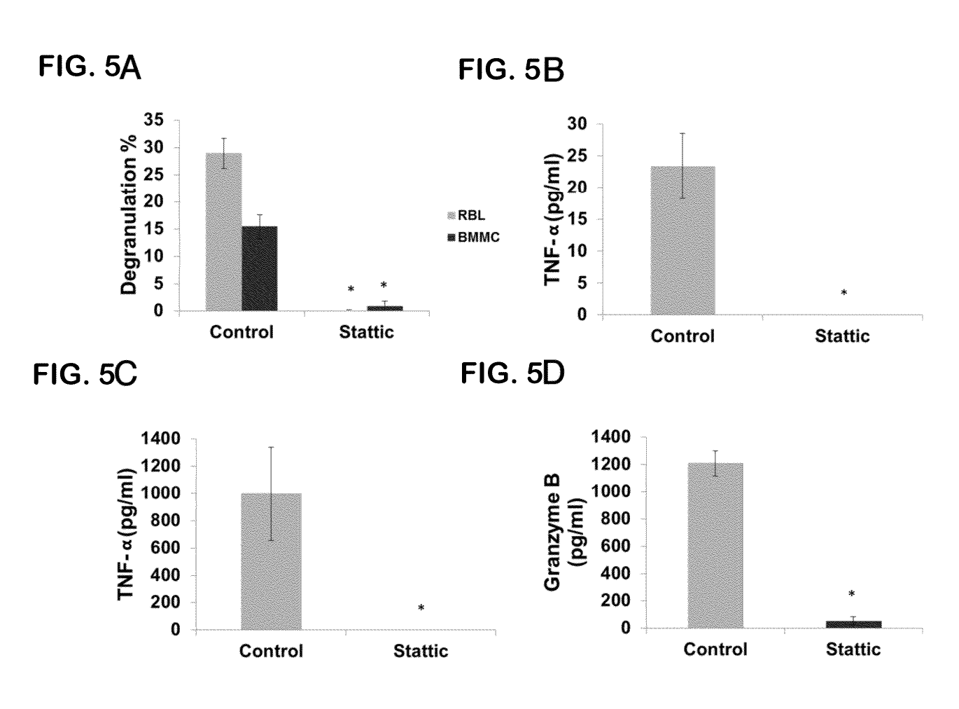

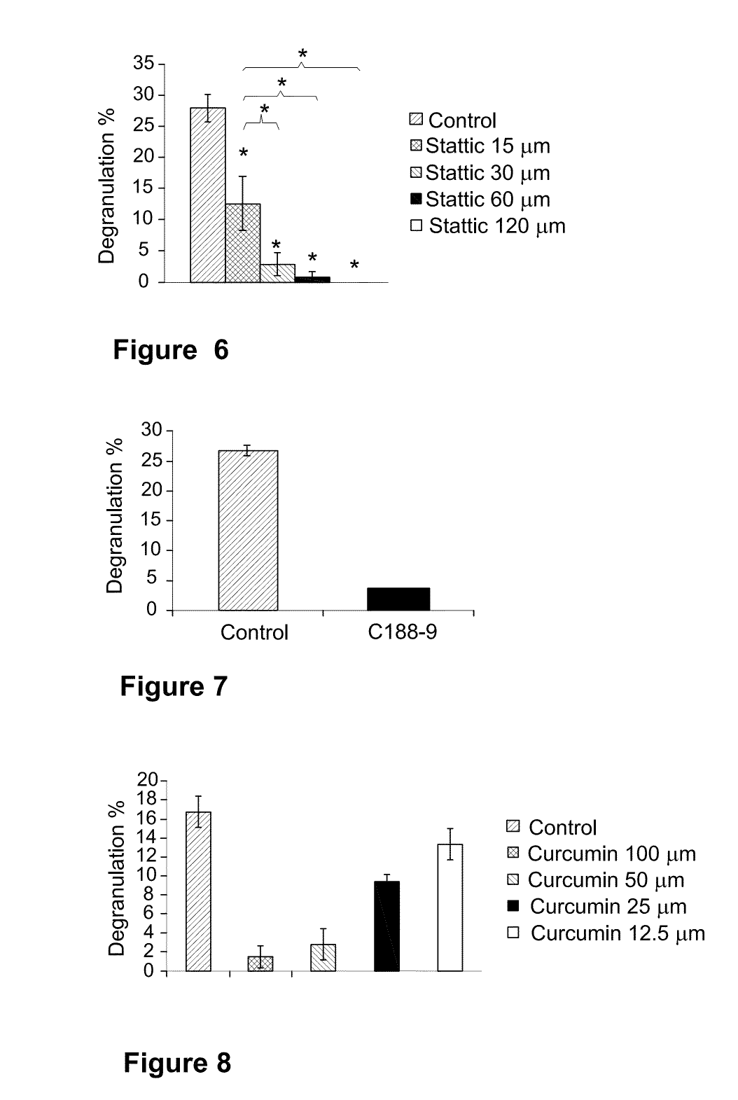

In order to further explore the effect of mitochondrial STAT3 on mast cell function, RBL cells were incubated for 20 min with 60 μM Stattic or DMSO alone (control) followed by determination of degranulation levels and TNF-α secretion. β-hexosaminidase release and TNF-α secretion in control cells were respectively 28.9±2.7% and 23.3±5.1 μg/ml (FIG. 5A-B). Incubation with Stattic completely abolished both mast cell 3-hexosaminidase release and TNF-α secretion (p<0.05). The degranulation was also totally abolished by 5 min incubation with Stattic (data not shown). This effect on IgE-Ag mediated β-hexosaminidase release of RBL cells was found to be concentration dependent (FIG. 6). Stattic treatment of immunologically activated BMMCs achieved similar results (FIGS. 5A&C). Granzyme B secretion was also greatly reduced (FIG. 5D). Further experiments with an additional STAT3 inhibitor C188-9 25 were carried out in order to confirm the selectivity of STAT3 inhibition in this system. As shown in FIG. 7, degranulation was also abolished in immunologically activated RBL cells treated with 250 μM C188-9 for 10 min. In addition 10 min incubation with curcumin an additional STAT3 inhibitor also completely abolished degranulation levels (FIG. 8).

### Example 4

### Mitochondrial STAT3 Serine 727 ERK Dependent Phosphorylation During Mast Cell Activation

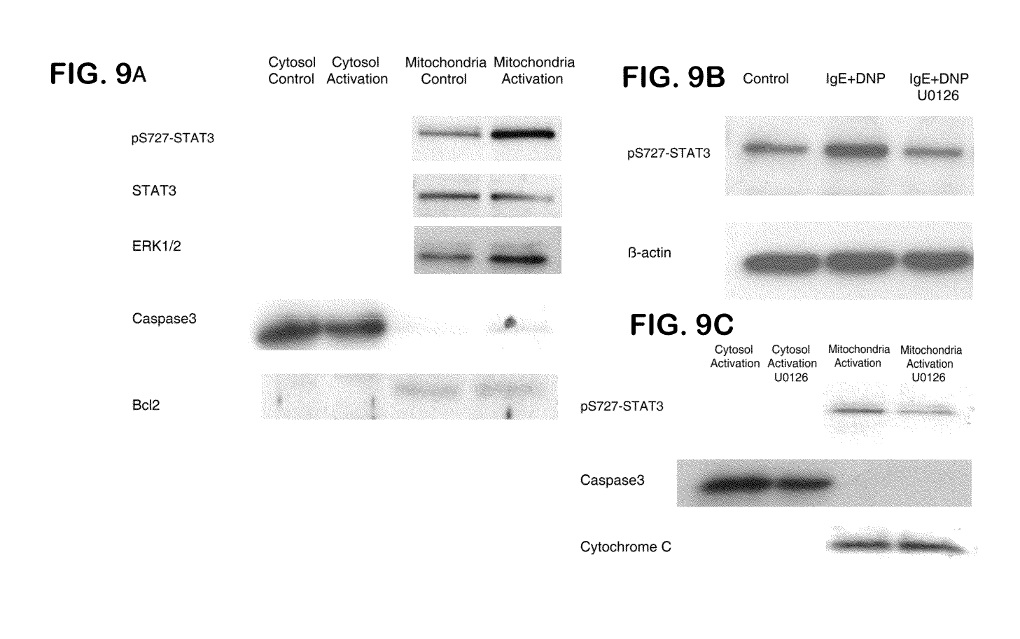

The inventors have previously reported that STAT3 undergoes phosphorylation on serine 727 during mast cell activation.¹⁵ However, the biological significance of this phosphorylation in mast cell activation was not fully understood. In order to determine if such phosphorylation occurred in the mitochondria following immunological activation in RBL cells, these cells were sensitized with IgE for 2 h and then triggered with DNP-BSA for 5 min, while quiescent cells were used as control. Mitochondria were then isolated from these cells and the levels of STAT3 serine 727 phosphorylation were determined by Western blot analysis. As shown in FIG. 9A, an enhancement in the level of mitochondrial STAT3 serine 727 phosphorylation was observed in the activated cells. To evaluate whether this phosphorylation resulted from STAT3 translocation into the mitochondria, the amount of STAT3 in the mitochondria was determined before and after cell activation. No change in total STAT3 levels was observed (FIG. 9A).

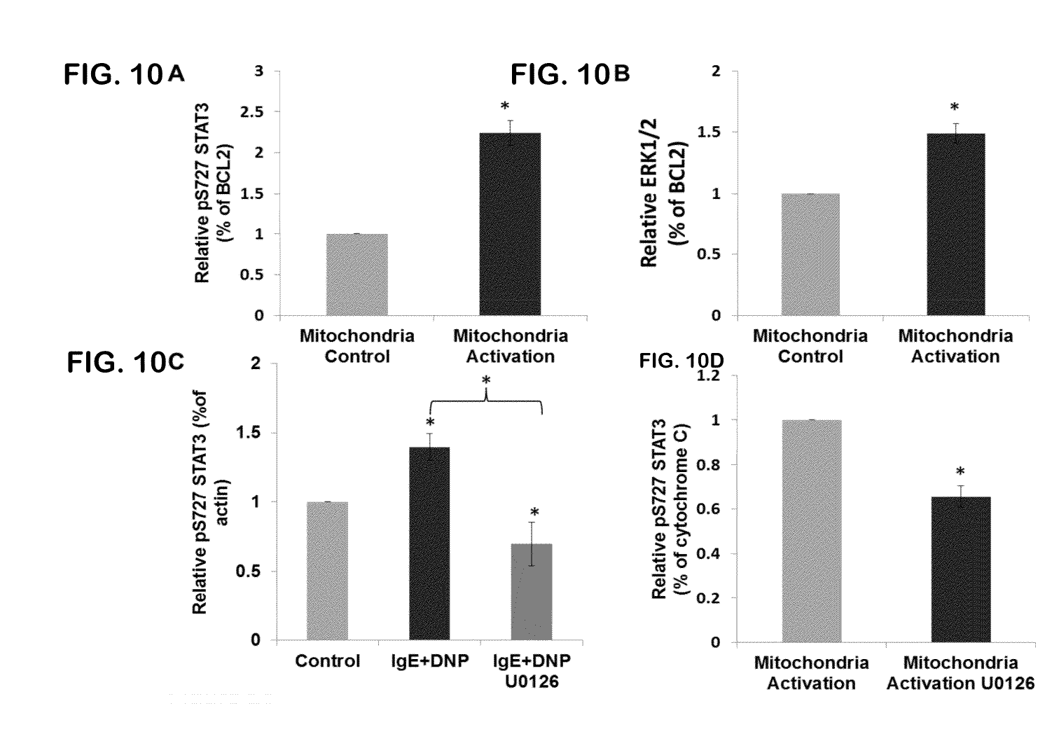

In order to determine whether ERK translocate to the mitochondria upon mast cell activation, the amount of ERK in the mitochondria was determined before and after 5 min of IgE-DNP activation in RBL cells. As shown in FIG. 9A, an enhancement in the level of mitochondrial ERK1/2 was observed in the activated cells. To further evaluate if mitochondrial ERK1/2 phosphorylates mitochondrial STAT3 on serine 727, cells were treated with the ERK pathway inhibitor, U0126. In a first stage RBL cells were either activated or not for 5 min in the presence of 40 μM U0126 dissolved in DMSO or DMSO alone (control) and the total cell level of phosphorylated STAT3 727 serine were determined by Western blot analysis. As shown in FIG. 9B a significant decrease in STAT3 727 serine phosphorylation was observed in the U0126 treated cells. In the second stage the immunologically activated RBL cells were fractionated into cytosol and mitochondria and the mitochondrial levels of phosphorylated ERK and STAT3 on serine 727 were determined by Western blot analysis. As shown in FIG. 9C, ERK1/2 inhibition was followed by a respective decrease in STAT3 727 serine phosphorylation. Densitometry quantification of the above Western blots is shown in FIG. 10.

### Example 5

### Inhibition of Mitochondrial ATP Production and Oxygen Consumption by U0126

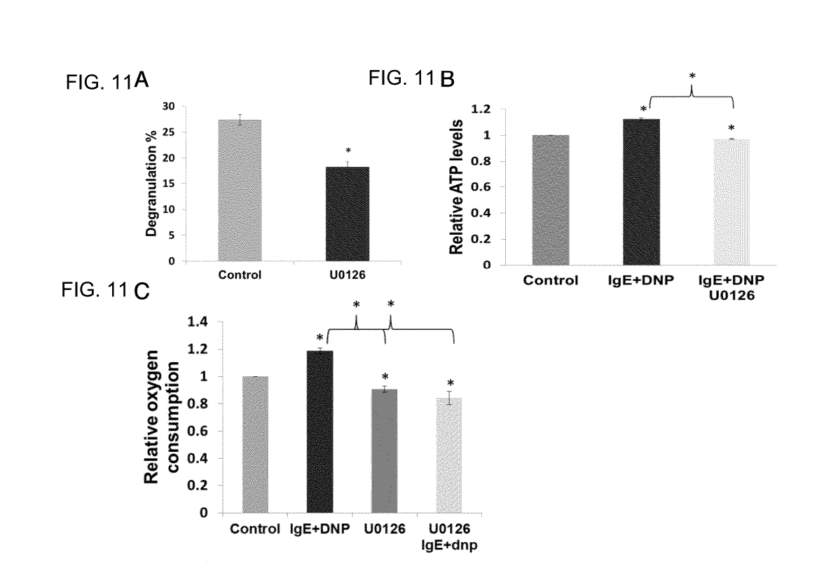

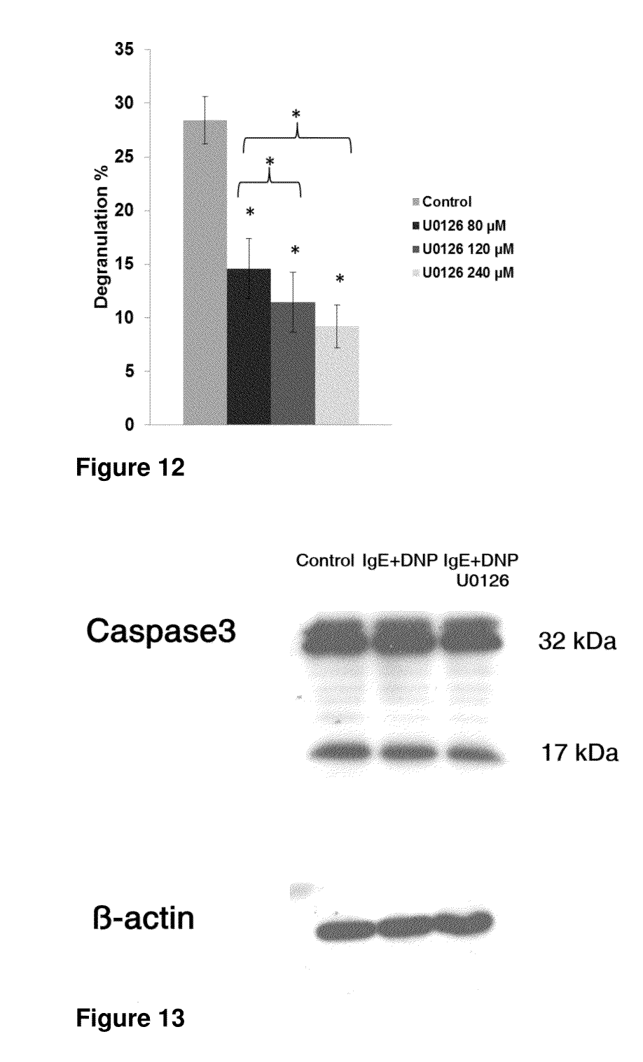

U0126 is a well-known inhibitor of mast cell degranulation.³⁶⁻⁴⁰ In order to determine whether part of the effect of U0126 on degranulation is mediated through inhibition of mitochondrial STAT3 serine 727 phosphorylation, the effect of ERK inhibition by U0126 on degranulation, ATP content and oxygen consumption in IgE-Ag stimulated RBL cells was next explored. As shown in FIG. 11A, 20 min incubation with 40 μM U0126 (dissolved in 100% DMSO) resulted in a 33.0±0.6% reduction in degranulation as compared to control (DMSO alone). Although higher concentrations of U0126 resulted in a more profound reduction in degranulation levels (FIG. 12), we used the minimum concentration that inhibited mast cell degranulation by more than 30% to insure specificity. The increase in ATP levels caused by immunological activation of RBL cells (12.2±1.0% increase, p<0.05) was reversed by 20 min pre-treatment with 40 μM U0126, resulting in a slight but significant decrease in ATP levels (3.0±0.4% decrease, p<0.05) (FIG. 11B). Furthermore, this inhibitor also caused a statistically significant reduction in oxygen consumption in both quiescent (9.4±2.4% decrease; p<0.05) and activated cells (15.8±4.9% decrease; p<0.05) (FIG. 11C). As shown in FIG. 13, the effect of U0126 was not due to apoptosis since no change in caspase 3 levels was observed in cells treated with U0126.

### Example 6

### The Role Played by PIAS3 in Mast Cell Function

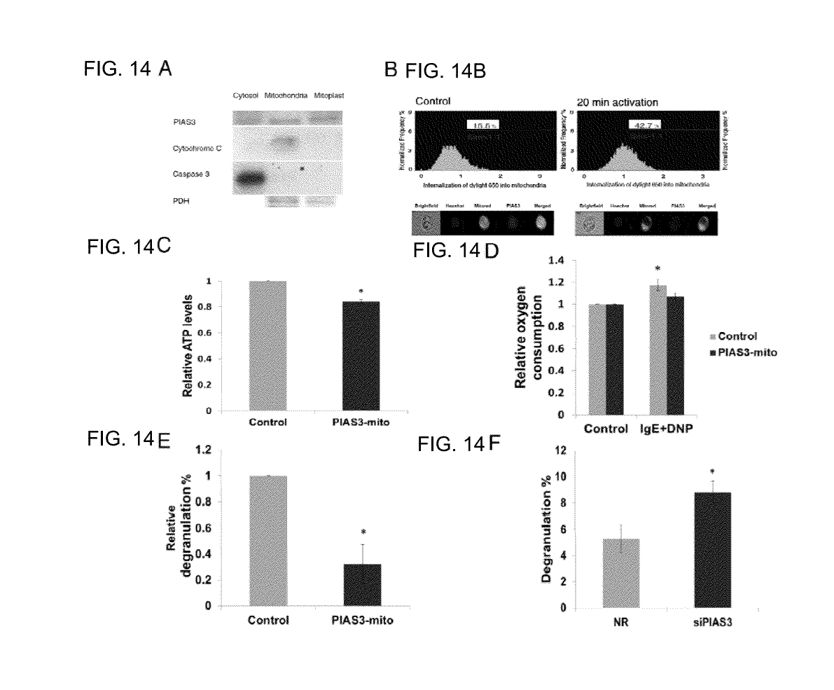

As mentioned previously, mast cell activation leads to the phosphorylation of STAT3 on the serine 727 moiety, followed by its association with PIAS3 which inhibits its transcriptional activity.^{9, 19} Since PIAS3 is known to be the main endogenous inhibitor of STAT3, the inventors next determined whether PIAS3 is localized in the mitochondria as well. Accordingly, RBL cells were fractionated into cytosol and mitochondria, and additionally, mitoplasts were prepared from the mitochondrial fraction by detergent treatment. PIAS3 content was determined by Western blot analysis. The purity of the cytosolic and mitochondrial fractions and of the mitoplasts was determined using cytochrome c antibody as an intermembrane space marker, caspase 3 antibody as a cytosol marker and pyruvate dehydrogenase antibody as a matrix marker. As shown in FIG. 14A, PIAS3 expression was detected in both the cytosolic and the mitochondrial fractions and in the mitoplasts. The presence of PIAS3 in the mitoplasts indicated that it is localized within the mitochondria and is not derived from either the contaminating cytoplasm or the mitochondrial intermembrane space. To further evaluate the expression of PIAS3 in the mitochondria following immunological activation, and to quantitatively assess whether PIAS3 is translocated to the mitochondria, ImageStream imaging flow cytometry was used. RBL cells were immunologically activated for 20 min and quiescent cells were used as control. Mitochondria were stained with mitotracker red; PIAS3 antibody was stained with dylight-650, and cell nuclei were stained with Hoechst. As shown in FIG. 14B, the localization of PIAS3 into the mitochondria increased nearly 3-fold following 20 min activation. Representative images of each treatment are shown.

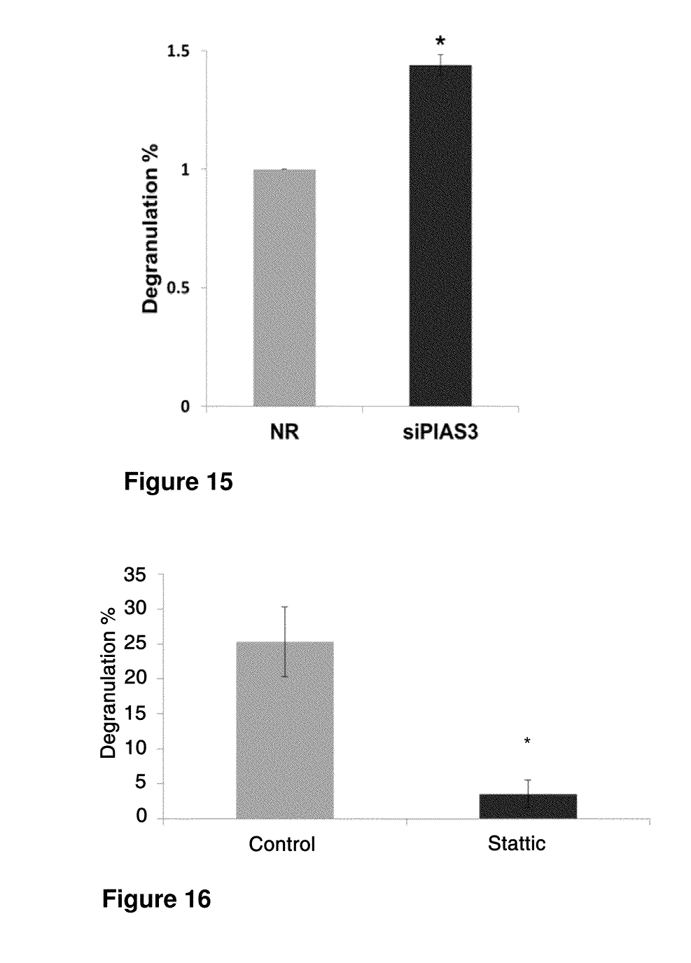

In order to determine the role played by mitochondrial PIAS3 in OXPHOS, RBL cells were transfected with a plasmid constructed with PIAS3 fused to a mitochondrial target sequence (PIAS3-mito) or empty plasmid (control). It should be noted that in some embodiments, the PIAS3-mito may comprise the amino acid sequence as denoted by SEQ ID NO. 16. The translocation of the recombinant PIAS3-mito into the mitochondria was confirmed by Western blot analysis. The transfected cells were immunologically activated for 5 min and ATP content and oxygen consumption were determined. As shown in FIG. 14C, transfection of PIAS3-mito resulted in a significant decrease in ATP levels of activated cells compared to control (15.9±1.6% decrease, p<0.05). Over expression of PIAS3 targeted to the mitochondria did not change the oxygen consumption in activated cells compared to activated control cells (empty plasmid) (FIG. 14D). In order to further explore the role played by mitochondrial PIAS3 in degranulation, RBL cells were transfected with PIAS3-mito or with the empty plasmid. As shown in FIG. 14E, transfection of PIAS3-mito resulted in a significant decrease in degranulation (67.5±15.0%, p<0.05). Complementary experiments were also performed to confirm that endogenous PIAS3 indeed regulates exocytosis in mast cells. BMMCs were transfected for 48 h with either siRNA against PIAS3 or with non-relevant siRNA as control. As shown in FIG. 14F, silencing of PIAS3 by siRNA led to a significant enhancement in degranulation levels of 66.4±8.6% (p<0.05). The silencing of PIAS3 with its specific siRNA was confirmed by Western blot analysis. Similar results were achieved using RBL cells as shown in FIG. 15.

### Example 7

### Mitochondrial STAT3 in Human Cord Blood-Derived Mast Cells

Next, the inventors determined whether in human mast cells mitochondrial STAT3 plays a similar role to that described above for murine mast cells. Human cord blood-derived mast cells (CBMC) were first sensitized with human myeloma IgE. Five min before activation with rabbit α-human-IgE, Stattic or DMSO alone were added and degranulation levels were determined after 30 min by measuring β-hexosaminidase release. As seen in FIG. 16, the degranulation seen in control cells (20.9±4.9) was completely abolished (3.5±1.9) by incubation with Stattic.

### Example 8

### In Vivo Experiments, STAT3's Role

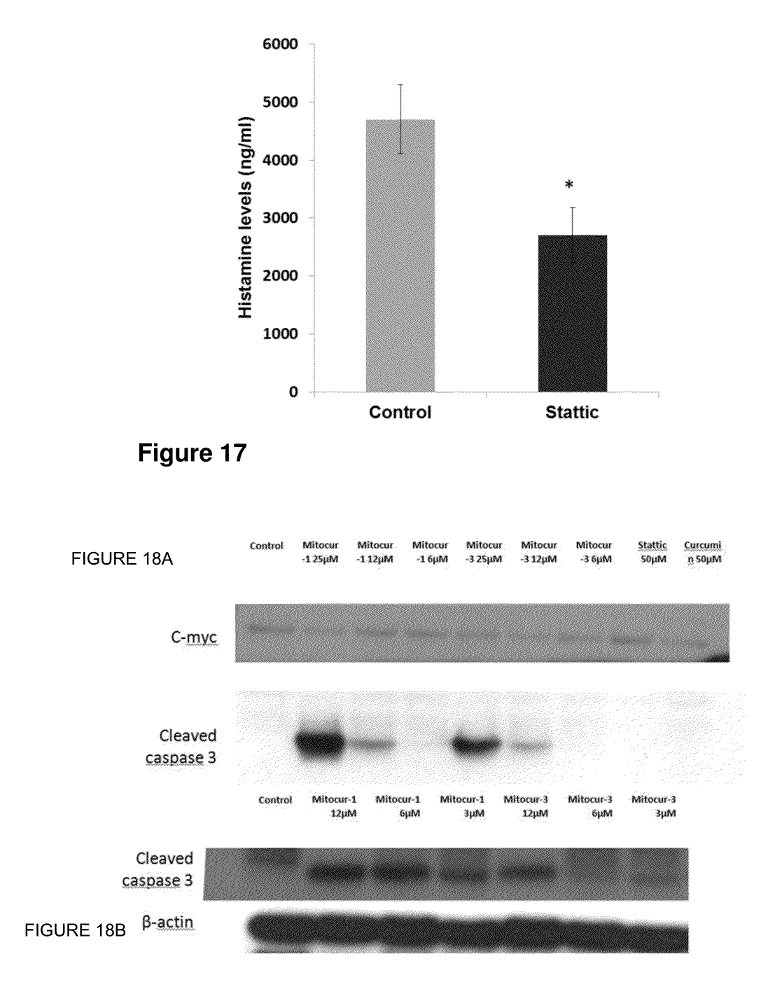

STAT3's role in vivo was investigated by evaluating the response to allergic stimulation in Stattic-treated mice. The passive systemic IgE-mediated anaphylaxis model was used. Mice were injected (i.v.) with IgE anti-DNP. Twenty four hours later they were injected (i.p.) with Stattic (80 mg/kg) or corn oil which was used as control. Three and a half hours later the mice were challenged with DNP-BSA for 1.5 minutes. Plasma was collected, and histamine levels were measured by competitive ELISA. Histamine secretion in Stattic-treated mice was 43% less than that in control mice (FIG. 17, p<0.05). Injection of a lower dose of Stattic (40 mg/kg) also resulted in a significant decrease (32.4%; p<0.05) in histamine secretion after two and a half hours (data not shown). These results demonstrate the critical role of STAT3 in mast cell-dependent allergic reactions.

### Example 9

### In Vivo Studies of TPP Conjugated to Stattic

DecylTPP is a lipophilic cation driven to the mitochondria by the mitochondrial membrane potential. Using custom synthesis of small molecules and fine chemicals TPP is conjugated to stattic to create a novel mitochondrial STAT3 inhibitor (MitoS), having the formula I:

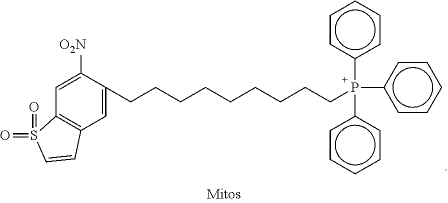

RBL cells are incubated with or without MitoS and STAT3 and transcriptional activity is determined on the RNA and protein levels. Furthermore the phosphorylation state of mitochondrial STAT3 is determined from isolated mitochondria following mast cell active. Cells are differentiated to mast cells (2-3 weeks); previous to immunogical activation (1-4 days) cells are incubated with or without MitoS and degranulation and secretion levels of cytokines IL-6, TNF-α, IL-4 and granzyme B are determined incubated with or without MitoS.

The correct solvent to inject MitoS is determined A range of concentrations of MitoS is injected at various time points after the passive systemic IgE-mediated anaphylaxis model will be used to measure the response. Blood is taken and histamine levels are determined by ELISA.

Dosage optimization is performed by finding the minimum dose still able to inhibit histamine release in the passive systemic IgE-mediated anaphylaxis assay. For systemic IgE-mediated anaphylaxis MitoS is injected. Afterwards mice are sensitized and rectal body temperature is recorded over 2 h every 10 min using a digital thermometer. To assess cutaneous anaphylaxis, mice are sensitized with injections of anti-DNP IgE into the ear pinna, challenged 24 h later with injections of DNP-HSA and ear thickness is measured over 6 h every 30 min using an engineer's micrometer.

For contact hypersensitivity investigation, following mitoS injection mice are sensitized epicutaneously on the shaved back with 0.5% acetone/olive oil (alternatively with 1% acetone/dibutylphtalate), challenged 6 days later on the ear with 0.2% DNFB (alternatively with 0.5% FITC) and ear swelling is monitored over 1 to 8 days using an engineer's micrometer.

MitoS is injected, BAL fluid is collected for determination of cytokines and chemokines by ELISA. Total cell counts is performed using a hemocytometer. For cytological examination, cytospin preparations are prepared using a Cytospin.

### Example 10

### Curcumine-Based Mitochondrial Targeted STAT Inhibitors

Mitocur-1, based on curcumin, which is a well known STAT3 inhibitor, conjugated to TPP has been next synthesized as described in Experimental procedures. The inventors further prepared curcumin analog, having a deletion of a methoxy substitution on the aryl groups, designated Mitocur-3, with this deletion.

The curcumin analogs of the invention, Mitocur-1 and Mitocur-3 are presented in Formula II (A) and Formula III (B), respectively.

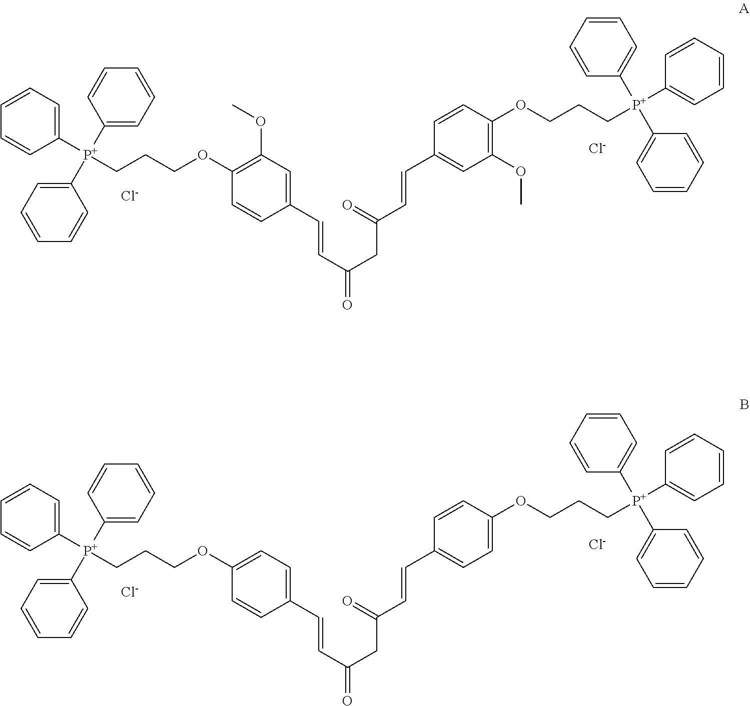 [SMILES: COc1cc(/C=C/C(=O)CC(=O)/C=C/c2ccc(OCCC[P+](c3ccccc3)(c3ccccc3)c3ccccc3)c(OC)c2)ccc1OCCC[P+](c1ccccc1)(c1ccccc1)c1ccccc1 ; [Cl-] ; [Cl-] ; O=C(/C=C/c1ccc(OCCC[P+](c2ccccc2)(c2ccccc2)c2ccccc2)cc1)CC(=O)/C=C/c1ccc(OCCC[P+](c2ccccc2)(c2ccccc2)c2ccccc2)cc1 ; [Cl-] ; [Cl-]]

### Example 11

### In Vitro Testing of Mitocur-1 and -3

To ensure that the mitochondrial targeted STAT3 inhibitors, Mitocur-1 and -3, do not affect STAT3's nuclear activity, RBL cells were incubated with various concentrations of Mitocur-1 or Mitocur-3 (Mitocur-1/3) for 3 h and levels of c-myc, a target gene of STAT3, were determined by Western blot analysis. The levels of cleaved caspase3 were measured as an indicator of apoptosis, which could occur as a result of inhibiting STAT3's target genes. As shown in FIG. 18A, no change in c-myc levels were observed using 6 μM of Mitocur-1/3 compared to control. In addition, no change in cleaved caspase levels were observed with 6 μM of Mitocur-1/3. Since higher concentrations reduced the levels of c-myc and enhanced the levels of cleaved caspase3, the inventors concluded that 6 μM Mitocur-1/3 or lower would be suitable for the present invention. As these molecules are intended to be used in a time frame greater than 3 h, RBL cells were incubated with the same concentrations of Mitocur-1/3 for 24 h and the levels of cleaved caspase3 were determined by Western blot analysis. As shown in FIG. 18B, a slight change in cleaved caspase3 levels was observed using 6 μM Mitocur-3 indicating a minor affect on STAT3 nuclear activity.

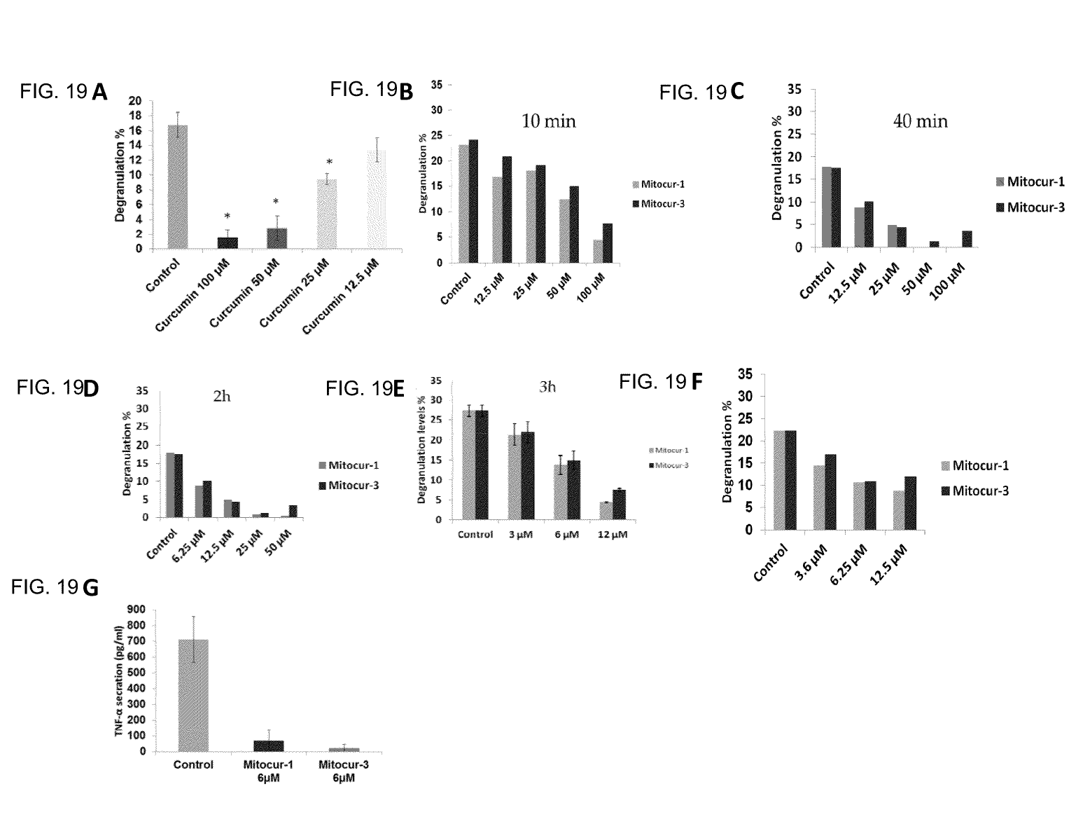

Before determining the inhibitors' effectiveness on mouse bone marrow derived mast cells (BMMC) and on cultured human mast cells (CBMS), the inventors determined whether curcumin alone could affect degranulation and calibrated the time and concentration dependence of Mitocur-1 and Mitocur-3 inhibition. RBL cells were incubated with curcumin (12.5, 25, 50 and 100 μM) for 10 min, a time frame which should not allow protein synthesis of STAT3 target genes, and β-hexosaminidase release was determined. As shown in FIG. 19A, curcumin significantly reduced degranulation. In order to determine the optimum time and concentration of Mitocur-1/3 for its effect on degranulation, RBL cells were incubated with the stated concentrations of Mitocur-1/3 for 10 min, 40 min, 2 h, 3 h and 24 h. As shown in FIG. 19B-19F, 6 μM of Mitocur-1/3, the concentration found not to affect STAT3 target genes or induce apoptosis, resulted in a maximum effect of over 50% reduction after 3 h incubation. In order to verify that this is the most suitable time and concentration for BMMC and CBMC studies, TNF-α secretion of RBL cells was determined as well. As shown in FIG. 19G, TNF-α secretion was completely abolished by Mitocur-1 and Mitocur-3.

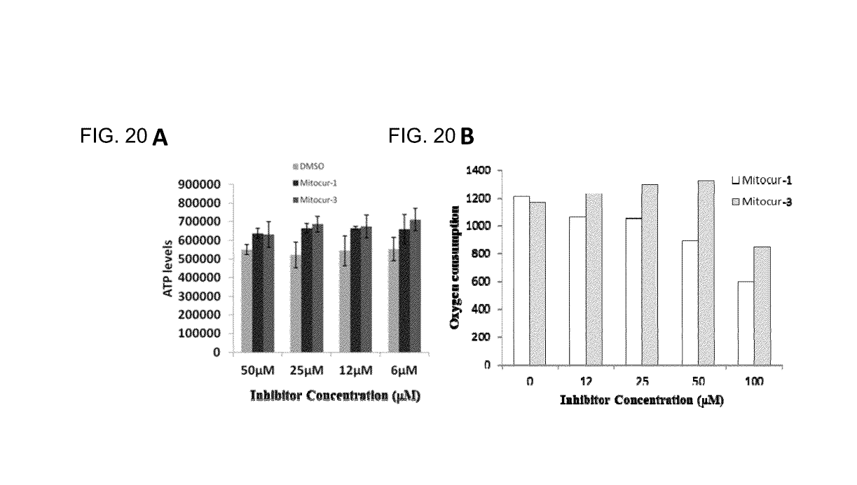

Since Mitocur-1 and 3 inhibit mitochondrial STAT3, it was important to find the concentrations of Mitocur-1 and 3 that would reduce degranulation levels with no effect, or only a mild effect, on ATP levels and oxygen consumption. The use of specific range of concentrations is important in reducing effects on other cells where mitochondrial STAT3 function is important. To assess the effect of Mitocur-1/3 on ATP levels and oxygen consumption, RBL cells were incubated with Mitocur-1/3 (6, 12, 25, 50 and 100 μM) for 3 h and ATP and oxygen consumption was measured. No significant reduction in ATP levels (FIG. 6A) or oxygen consumption (FIG. 20B) were observed when 6-50 μM of Mitocur-1/3. 100 μM Mitocur-1/3 reduced both oxygen consumption (FIG. 20B) and ATP levels (data not shown), due to STAT3 inhibition on mitochondrial function.

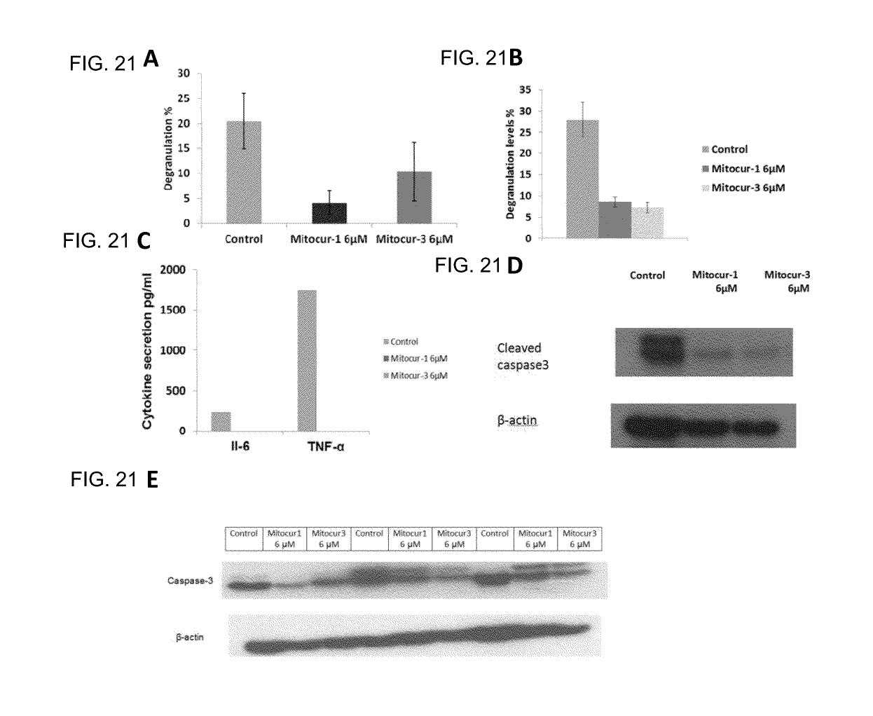

Therefore, the effect of Mitocur-1 and -3 on degranulation and cytokine secretion, represented by IL-6 and TNF-α was next evaluated, in BMMC using 6 μM of Mitocur-1/3 and 3 h or 24 h incubation. As shown in FIG. 21A, 21B, Mitocur-1 and -3 reduced the degranulation levels after 3 h and 24 h incubation and totally abolished cytokine secretion in immunologically activated BMMC (FIG. 21C). Granzyme-B secretion was also abolished by treatment with Mitocur-1/3 (data not shown). The levels of cleaved caspase3 were determined by Western blot analysis. As shown in FIG. 21D, no change in cleaved caspase3 levels were observed, indicating that the used mitochondrial-targeted STAT3 inhibitor has no effect on apoptosis.

### Example 12

### In Vivo Testing of Mitocur-1 and -3

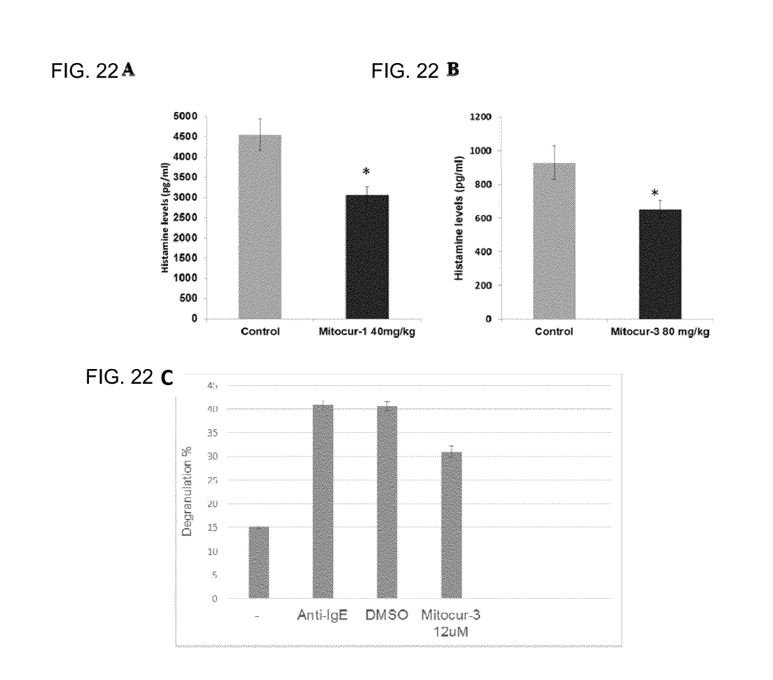

Mitocur-1/3 effectiveness in vivo was investigated by evaluating the response to allergic stimulation in Mitocur-1/3-treated mice. The passive systemic IgE-mediated anaphylaxis model was used. Mice were injected (i.v.) with IgE anti-DNP. Twenty four hours later they were injected (i.p.) with Mitocur-1 (80 mg/kg) and Mitocur-3 (40 mg/kg). Corn oil was used as control. Three and a half hours later the mice were challenged with DNP-BSA for 1.5 minutes. Plasma was collected, and histamine levels were measured by competitive ELISA. Histamine secretion in Mitocur-1-treated mice was 35% less than that in control mice (FIG. 22A, p<0.05). Injection of Mitocur-3 (40 mg/kg) also resulted in a significant decrease (32.4%; p<0.05) in histamine secretion (FIG. 22B).

Next the effect of Mitocur-3 on degranulation in human cord blood mast cells (CBMC) was determined after 3 h incubation with 12 μM of mitocur-3. As shown in FIG. 22C Mitocur-3 successfully reduced degranulation levels.

1. A composition of matter comprising at least one signal transducer and activator of transcription factor 3 (STAT3) inhibiting moiety or any vehicle, matrix, nano- or micro-particle comprising the same, associated with at least one mitochondrial targeting moiety.

2. The composition of matter according to claim 1, further comprising at least one mast cell (MC) targeting moiety.

3. The composition of matter according to claim 1, wherein said at least one mitochondrial targeting moiety is at least one of a lipohilic cation/s and/or at least one mitochondrial targeting sequence/s.

4. The composition of matter according to claim 3, wherein said lipophilic cation is Triphenylphosphonium (TPP).

5. The composition of matter according to claim 1, wherein said at least one STAT3 inhibiting moiety is at least one of a small molecule based moiety, a nucleic acid based moiety, an amino acid based moiety and any combinations thereof.

6. The composition of matter according to claim 5, wherein said at least one STAT3 inhibiting moiety is any one of STAT3 Inhibitor V, 6-Nitrobenzo[b]thiophene 1,1-dioxide (Stattic); (1E,6E)-1,7-Bis(4-hydroxy-3-methoxyphenyl)-1,6-heptadiene-3,5-dione (curcumin); STAT3 Inhibitor XIII, C188-9; Protein Inhibitor Of Activated STAT, 3 (PIAS3), any derivatives, analogs or any combinations thereof or any vehicle, matrix, nano- or micro-particle comprising the same.

7. The composition of matter according to claim 6, comprising 6-Nitrobenzo[b]thiophene 1,1-dioxide associated with TPP.

8. The composition of matter according to claim 6, comprising liposomes encapsulating in their intraliposomal core at least one 6-Nitrobenzo[b]thiophene 1,1-dioxide and having at least one TPP associated onto the outer liposomal surface.

9. The composition of matter according to claim 6, wherein said at least one STAT3 inhibiting moiety is at least one PIAS3 or any fragments or peptides thereof, said PIAS3 is attached to or associated with at least one mitochondrial targeting peptide.

10. A pharmaceutical composition comprising a pharmaceutically acceptable carrier and, as an active ingredient, at least one STAT3 inhibiting moiety or any vehicle, matrix, nano- or micro-particle comprising the same, associated with at least one mitochondrial targeting moiety, or any composition of matter comprising the same according to claim 1, said active ingredient is present in an amount effective for inhibiting and/or decreasing mast cell (MC) degranulation, while retaining STAT3 nuclear function's.

11. The pharmaceutical composition according to claim 10, wherein said at least one mitochondrial targeting moiety is at least one of a lipohilic cation and/or at least one mitochondrial targeting sequence.

12. The pharmaceutical composition according to claim 10, wherein said lipophilic cation is Triphenylphosphonium (TPP) and wherein said at least one STAT3 inhibiting moiety is at least one of a small organic molecule based moiety, a nucleic acid based moiety, an amino acid based moiety and any combinations thereof.

13. The pharmaceutical composition according to claim 12, wherein said at least one STAT3 inhibiting moiety is any one of STAT3 Inhibitor V, 6-Nitrobenzo[b]thiophene 1,1-dioxide (Stattic); (1E,6E)-1,7-Bis(4-hydroxy-3-methoxyphenyl)-1,6-heptadiene-3,5-dione (curcumin); STAT3 Inhibitor XIII, C188-9; Protein Inhibitor Of Activated STAT, 3 (PIAS3), any derivatives, analogs or any combinations thereof or any vehicle, matrix, nano- or micro-particle comprising the same.

14. The pharmaceutical composition according to claim 12, wherein said at least one STAT3 inhibiting moiety is any one of -Nitrobenzo[b]thiophene 1,1-dioxide associated with TPP, (1E,6E)-1,7-Bis(4-hydroxy-3-methoxyphenyl)-1,6-heptadiene-3,5-dione or derivatives thereof associated with TPP and 6-Nitrobenzo[b]thiophene 1,1-dioxide and having at least one TPP associated onto the outer liposomal surface.

15. The pharmaceutical composition according to claim 10, wherein said composition is adapted for pulmonary delivery.

16. A method for inhibiting, reducing or decreasing MC activation, the method comprising contacting mast cells with an effective amount of at last one STAT3 inhibiting moiety or any vehicle, matrix, nano- or micro-particle comprising the same, associated with at least one mitochondrial targeting moiety, or with any composition comprising the same.

17. The method according to claim 16, for decreasing mast cell activation in a subject in need thereof, the method comprising administering said subject an effective amount of at last one STAT3 inhibiting moiety or any vehicle, matrix, nano- or micro-particle comprising the same, associated with at least one mitochondrial targeting moiety, or with any composition comprising the same.

18. The method according to claim 16, wherein said at least one mitochondrial targeting moiety is at least one of a lipohilic cation and/or at least one mitochondrial targeting sequence, and said at least one STAT3 inhibiting moiety is at least one of a small molecule based moiety, a nucleic acid based moiety, an amino acid based moiety and any combinations thereof.

19. A method for treating a subject suffering from a medical condition associated with or induced by mast cell activation, the method comprising administering to said subject a therapeutically effective amount of at last one STAT3 inhibiting moiety or any vehicle, matrix, nano- or micro-particle comprising the same, associated with at least one mitochondrial targeting moiety, or with any composition comprising the same according to claim 10.

20. The method according to claim 19, wherein said medical condition associated with or induced by mast cell activation is any one of allergic reaction, asthma, eczema, dermatitis, allergic rhinitis allergic conjunctivitis, anaphylaxis, mastocytosis and mast cell tumors.

21. The method according to claim 20, wherein said at least one mitochondrial targeting moiety is at least one of a lipohilic cation and/or at least one mitochondrial targeting sequence, and said at least one STAT3 inhibiting moiety is at least one of a small molecule based moiety, a nucleic acid based moiety, an amino acid based moiety and any combinations thereof.


SEQUENCE LISTING (23 sequences)

SEQ ID NO: 1 - protein, 770 aa, Homo sapiens
MAQWNQLQQLDTRYLEQLHQLYSDSFPMELRQFLAPWIESQDWAYAASKESHATLVFHNLLGEIDQQYSRFLQESNVLYQHNLRRIKQFLQSRYLEKPMEIARIVARCLWEESRLLQTAATAAQQGGQANHPTAAVVTEKQQMLEQHLQDVRKRVQDLEQKMKVVENLQDDFDFNYKTLKSQGDMQDLNGNNQSVTRQKMQQLEQMLTALDQMRRSIVSELAGLLSAMEYVQKTLTDEELADWKRRQQIACIGGPPNICLDRLENWITSLAESQLQTRQQIKKLEELQQKVSYKGDPIVQHRPMLEERIVELFRNLMKSAFVVERQPCMPMHPDRPLVIKTGVQFTTKVRLLVKFPELNYQLKIKVCIDKDSGDVAALRGSRKFNILGTNTKVMNMEESNNGSLSAEFKHLTLREQRCGNGGRANCDASLIVTEELHLITFETEVYHQGLKIDLETHSLPVVVISNICQMPNAWASILWYNMLTNNPKNVNFFTKPPIGTWDQVAEVLSWQFSSTTKRGLSIEQLTTLAEKLLGPGVNYSGCQITWAKFCKENMAGKGFSFWVWLDNIIDLVKKYILALWNEGYIMGFISKERERAILSTKPPGTFLLRFSESSKEGGVTFTWVEKDISGKTQIQSVEPYTKQQLNNMSFAEIIMGYKIMDATNILVSPLVYLYPDIPKEEAFGKYCRPESQEHPEADPGSAAPYLKTKFICVTPTTCSNTIDLPMSPRTLDSLMQFGNNGEGAEPSAGGQFESLTFDMELTSECATSPM

SEQ ID NO: 2 - protein, 769 aa, Homo sapiens
MAQWNQLQQLDTRYLEQLHQLYSDSFPMELRQFLAPWIESQDWAYAASKESHATLVFHNLLGEIDQQYSRFLQESNVLYQHNLRRIKQFLQSRYLEKPMEIARIVARCLWEESRLLQTAATAAQQGGQANHPTAAVVTEKQQMLEQHLQDVRKRVQDLEQKMKVVENLQDDFDFNYKTLKSQGDMQDLNGNNQSVTRQKMQQLEQMLTALDQMRRSIVSELAGLLSAMEYVQKTLTDEELADWKRRQQIACIGGPPNICLDRLENWITSLAESQLQTRQQIKKLEELQQKVSYKGDPIVQHRPMLEERIVELFRNLMKSAFVVERQPCMPMHPDRPLVIKTGVQFTTKVRLLVKFPELNYQLKIKVCIDKDSGDVAALRGSRKFNILGTNTKVMNMEESNNGSLSAEFKHLTLREQRCGNGGRANCDASLIVTEELHLITFETEVYHQGLKIDLETHSLPVVVISNICQMPNAWASILWYNMLTNNPKNVNFFTKPPIGTWDQVAEVLSWQFSSTTKRGLSIEQLTTLAEKLLGPGVNYSGCQITWAKFCKENMAGKGFSFWVWLDNIIDLVKKYILALWNEGYIMGFISKERERAILSTKPPGTFLLRFSESSKEGGVTFTWVEKDISGKTQIQSVEPYTKQQLNNMSFAEIIMGYKIMDATNILVSPLVYLYPDIPKEEAFGKYCRPESQEHPEADPGAAPYLKTKFICVTPTTCSNTIDLPMSPRTLDSLMQFGNNGEGAEPSAGGQFESLTFDMELTSECATSPM

SEQ ID NO: 3 - protein, 722 aa, Homo sapiens
MAQWNQLQQLDTRYLEQLHQLYSDSFPMELRQFLAPWIESQDWAYAASKESHATLVFHNLLGEIDQQYSRFLQESNVLYQHNLRRIKQFLQSRYLEKPMEIARIVARCLWEESRLLQTAATAAQQGGQANHPTAAVVTEKQQMLEQHLQDVRKRVQDLEQKMKVVENLQDDFDFNYKTLKSQGDMQDLNGNNQSVTRQKMQQLEQMLTALDQMRRSIVSELAGLLSAMEYVQKTLTDEELADWKRRQQIACIGGPPNICLDRLENWITSLAESQLQTRQQIKKLEELQQKVSYKGDPIVQHRPMLEERIVELFRNLMKSAFVVERQPCMPMHPDRPLVIKTGVQFTTKVRLLVKFPELNYQLKIKVCIDKDSGDVAALRGSRKFNILGTNTKVMNMEESNNGSLSAEFKHLTLREQRCGNGGRANCDASLIVTEELHLITFETEVYHQGLKIDLETHSLPVVVISNICQMPNAWASILWYNMLTNNPKNVNFFTKPPIGTWDQVAEVLSWQFSSTTKRGLSIEQLTTLAEKLLGPGVNYSGCQITWAKFCKENMAGKGFSFWVWLDNIIDLVKKYILALWNEGYIMGFISKERERAILSTKPPGTFLLRFSESSKEGGVTFTWVEKDISGKTQIQSVEPYTKQQLNNMSFAEIIMGYKIMDATNILVSPLVYLYPDIPKEEAFGKYCRPESQEHPEADPGSAAPYLKTKFICVTPFIDAVWK

SEQ ID NO: 4 - protein, 770 aa, Mus musculus
MAQWNQLQQLDTRYLEQLHQLYSDSFPMELRQFLAPWIESQDWAYAASKESHATLVFHNLLGEIDQQYSRFLQESNVLYQHNLRRIKQFLQSRYLEKPMEIARIVARCLWEESRLLQTAATAAQQGGQANHPTAAVVTEKQQMLEQHLQDVRKRVQDLEQKMKVVENLQDDFDFNYKTLKSQGDMQDLNGNNQSVTRQKMQQLEQMLTALDQMRRSIVSELAGLLSAMEYVQKTLTDEELADWKRRQQIACIGGPPNICLDRLENWITSLAESQLQTRQQIKKLEELQQKVSYKGDPIVQHRPMLEERIVELFRNLMKSAFVVERQPCMPMHPDRPLVIKTGVQFTTKVRLLVKFPELNYQLKIKVCIDKDSGDVAALRGSRKFNILGTNTKVMNMEESNNGSLSAEFKHLTLREQRCGNGGRANCDASLIVTEELHLITFETEVYHQGLKIDLETHSLPVVVISNICQMPNAWASILWYNMLTNNPKNVNFFTKPPIGTWDQVAEVLSWQFSSTTKRGLSIEQLTTLAEKLLGPGVNYSGCQITWAKFCKENMAGKGFSFWVWLDNIIDLVKKYILALWNEGYIMGFISKERERAILSTKPPGTFLLRFSESSKEGGVTFTWVEKDISGKTQIQSVEPYTKQQLNNMSFAEIIMGYKIMDATNILVSPLVYLYPDIPKEEAFGKYCRPESQEHPEADPGSAAPYLKTKFICVTPTTCSNTIDLPMSPRTLDSLMQFGNNGEGAEPSAGGQFESLTFDMDLTSECATSPM

SEQ ID NO: 5 - protein, 770 aa, Mus musculus; identical to SEQ ID NO: 4

SEQ ID NO: 6 - protein, 769 aa, Mus musculus
MAQWNQLQQLDTRYLEQLHQLYSDSFPMELRQFLAPWIESQDWAYAASKESHATLVFHNLLGEIDQQYSRFLQESNVLYQHNLRRIKQFLQSRYLEKPMEIARIVARCLWEESRLLQTAATAAQQGGQANHPTAAVVTEKQQMLEQHLQDVRKRVQDLEQKMKVVENLQDDFDFNYKTLKSQGDMQDLNGNNQSVTRQKMQQLEQMLTALDQMRRSIVSELAGLLSAMEYVQKTLTDEELADWKRRQQIACIGGPPNICLDRLENWITSLAESQLQTRQQIKKLEELQQKVSYKGDPIVQHRPMLEERIVELFRNLMKSAFVVERQPCMPMHPDRPLVIKTGVQFTTKVRLLVKFPELNYQLKIKVCIDKDSGDVAALRGSRKFNILGTNTKVMNMEESNNGSLSAEFKHLTLREQRCGNGGRANCDASLIVTEELHLITFETEVYHQGLKIDLETHSLPVVVISNICQMPNAWASILWYNMLTNNPKNVNFFTKPPIGTWDQVAEVLSWQFSSTTKRGLSIEQLTTLAEKLLGPGVNYSGCQITWAKFCKENMAGKGFSFWVWLDNIIDLVKKYILALWNEGYIMGFISKERERAILSTKPPGTFLLRFSESSKEGGVTFTWVEKDISGKTQIQSVEPYTKQQLNNMSFAEIIMGYKIMDATNILVSPLVYLYPDIPKEEAFGKYCRPESQEHPEADPGAAPYLKTKFICVTPTTCSNTIDLPMSPRTLDSLMQFGNNGEGAEPSAGGQFESLTFDMDLTSECATSPM

SEQ ID NO: 7 - protein, 722 aa, Mus musculus; identical to SEQ ID NO: 3

SEQ ID NO: 8 - protein, 29 aa, Artificial Sequence
  Synthetic
MSVLTPLLLRGLTGSARRLPVPRAKIHSL

SEQ ID NO: 9 - protein, 25 aa, Artificial Sequence
  Synthetic
MLSLRQSIRFFKPATRTLCSSRYLL

SEQ ID NO: 10 - protein, 6 aa, Artificial Sequence
  Synthetic
PYLKTK

SEQ ID NO: 11 - protein, 18 aa, Artificial Sequence
  Synthetic
PYLKTKAAVLLPVLLAAP

SEQ ID NO: 12 - DNA, 1260 bp, Mus musculus
actgtgacctggtattcagactccctgaacatgagcactgtgaacttccctgccctcggttctgaactcaaggtcaccaccagccaagtgaccagctggggcaagtcagccaagaacttcacatgccacgtgacacatcctccatcattcaacgaaagtaggactatcctagttcgacctgtcaacatcactgagcccaccttggagctactccattcatcctgcgaccccaatgcattccactccaccatccagctgtactgcttcatttatggccacatcctaaatgatgtctctgtcagctggctaatggacgatcgggagataactgatacacttgcacaaactgttctaatcaaggaggaaggcaaactagcctctacctgcagtaaactcaacatcactgagcagcaatggatgtctgaaagcaccttcacctgcaaggtcacctcccaaggcgtagactatttggcccacactcggagatgcccagatcatgagccacggggtgtgattacctacctgatcccacccagccccctggacctgtatcaaaacggtgctcccaagcttacctgtctggtggtggacctggaaagcgagaagaatgtcaatgtgacgtggaaccaagagaagaagacttcagtctcagcatcccagtggtacactaagcaccacaataacgccacaactagtatcacctccatcctgcctgtagttgccaaggactggattgaaggctacggctatcagtgcatagtggaccaccctgattttcccaagcccattgtgcgttccatcaccaagaccccaggccagcgctcagcccccgaggtatatgtgttcccaccaccagaggaggagagcgaggacaaacgcacactcacctgtttgatccagaacttcttccctgaggatatctctgtgcagtggctgggggatggcaaactgatctcaaacagccagcacagtaccacaacacccctgaaatccaatggctccaatcaaggcttcttcatcttcagtcgcctagaggtcgccaagacactctggacacagagaaaacagttcacctgccaagtgatccatgaggcacttcagaaacccaggaaactggagaaaacaatatccacaagccttggtaacacctccctccgtccctcctaggcctccatgtagctgtggtggggaaggtggatgacagacatccgctcactgttgtaacaccaggaagctaccccaataaacactcagtgcctg

SEQ ID NO: 13 - protein, 25 aa, Artificial Sequence
  Synthetic
YGRKKRRQRGFCDGFYACYKDVPYL

SEQ ID NO: 14 - protein, 628 aa, Homo sapiens
MAELGELKHMVMSFRVSELQVLLGFAGRNKSGRKHELLAKALHLLKSSCAPSVQMKIKELYRRRFPRKTLGPSDLSLLSLPPGTSPVGSPGPLAPIPPTLLAPGTLLGPKREVDMHPPLPQPVHPDVTMKPLPFYEVYGELIRPTTLASTSSQRFEEAHFTFALTPQQVQQILTSREVLPGAKCDYTIQVQLRFCLCETSCPQEDYFPPNLFVKVNGKLCPLPGYLPPTKNGAEPKRPSRPINITPLARLSATVPNTIVVNWSSEFGRNYSLSVYLVRQLTAGTLLQKLRAKGIRNPDHSRALIKEKLTADPDSEVATTSLRVSLMCPLGKMRLTVPCRALTCAHLQSFDAALYLQMNEKKPTWTCPVCDKKAPYESLIIDGLFMEILSSCSDCDEIQFMEDGSWCPMKPKKEASEVCPPPGYGLDGLQYSPVQGGDPSENKKKVEVIDLTIESSSDEEDLPPTKKHCSVTSAAIPALPGSKGVLTSGHQPSSVLRSPAMGTLGGDFLSSLPLHEYPPAFPLGADIQGLDLFSFLQTESQHYGPSVITSLDEQDALGHFFQYRGTPSHFLGPLAPTLGSSHCSATPAPPPGRVSSIVAPGGALREGHGGPLPSGPSLTGCRSDIISLD

SEQ ID NO: 15 - protein, 619 aa, Mus musculus
MVMSFRVSELQVLLGFAGRNKSGRKHELLAKALHLLKSSCAPSVQMKIKELYRRRFPRKTLGPSDLSLLSLPPGTSPVGSPGPLAPIPPTLLTPGTLLGPKREVDMHPPLPQPVHPDVTMKPLPFYEVYGELIRPTTLASTSSQRFEEAHFTFALTPQQLQQILTSREVLPGAKCDYTIQVQLRFCLCETSCPQEDYFPPNLFVKVNGKLCPLPGYLPPTKNGAEPKRPSRPINITPLARLSATVPNTIVVNWSSEFGRNYSLSVYLVRQLTAGTLLQKLRAKGIRNPDHSRALIKEKLTADPDSEVATTSLRVSLMCPLGKMRLTVPCRALTCAHLQSFDAALYLQMNEKKPTWTCPVCDKKAPYESLIIDGLFMEILNSCSDCDEIQFMEDGSWCPMKPKKEASEVCPPPGYGLDGLQYSAVQEGIQPESKKRVEVIDLTIESSSDEEDLPPTKKHCPVTSAAIPALPGSKGALTSGHQPSSVLRSPAMGTLGSDFLSSLPLHEYPPAFPLGADIQGLDLFSFLQTESQHYGPSVITSLDEQDTLGHFFQYRGTPSHFLGPLAPTLGSSHRSSTPAPPPGRVSSIVAPGSSLREGHGGPLPSGPSLTGCRSDVISLD

SEQ ID NO: 16 - protein, 614 aa, Artificial Sequence
  Synthetic
MSVLTPLLLRGLTGSARRLPVPRAKIHSLLQVDSFRVSELQVLLGFAGRNKSGRKHELLAKALHLLKSSCAPSVQMKIKELYRRRFPRKTLGPSDLSLLSLPPGTSPPVHPDVTMKPLPFYEVYGELIRPTTLASTSSQRFEEAHFTFALTPQQLQQILTSREVLPGAKCDYTIQVQLRFCLCETSCPQEDYFPPNLFVKVNGKLCPLPGYLPPTKNGAEPKRPSRPINITPLARLSATVPNTIVVNWSSEFGRNYSLSVYLVRQLTAGTLLQKLRAKGIRNPDHSRALIKEKLTADPDSEVATTSLRVSLMCPLGKMRLTVPCRALTCAHLQSFDAALYLQMNEKKPTWTCPVCDKKAPYESLIIDGLFMEILNSCSDCDEIQFMEDGSWCPMKPKKEASEVCPPPGYGLDGLQYSAVQEGIQPESKKRVEVIDLTIESSSDEEDLPPTKKHCPVTSAAIPALPGSKGALTSGHQPSSVLRSPAMGTLGSDFLSSLPLHEYPPAFPLGADIQGLDLFSFLQTESQHYGPSVITSLDEQDTLGHFFQYRGTPSHFLGPLAPTLGSSHRSSTPAPPPGRVSSIVAPGSSLREGHGGPLPSGPSLTGCRSDVISLD

SEQ ID NO: 17 - DNA, 1845 bp, Artificial Sequence
  Synthetic
atgtccgtcctgacgccgctgctgctgcggggcttgacaggctcggcccggcggctcccagtgccgcgcgccaagatccattcgttgctgcaggtcgacagtttccgagtgtctgagctccaggtgctcctcggcttcgctggcaggaacaagagtgggcggaaacacgagctgctggccaaggccctgcacctcctcaagtctagctgcgcccccagcgtccagatgaagatcaaagaactttatcgcaggcgctttccccggaagaccctggggccttctgatctctccttgctttctttgccccctggcacctctcctcctgtgcaccccgatgtcaccatgaagccactgcccttctatgaagtctatggggagctcatccgacccaccacccttgcgtccacctccagccagaggttcgaggaagcccacttcaccttcgcgctcactccccagcagctgcagcagattctcacgtccagggaagttctgccaggagccaagtgtgattacaccatacaagtgcagctcagattctgtctctgtgagaccagctgccctcaggaggactatttcccccctaacctctttgttaaggttaatgggaaactctgccccctgccgggttacctccctccaaccaagaatggagctgagcccaagaggcccagccgtccgatcaacatcacacccttggctcgactctcagccactgtccccaacaccatcgttgttaattggtcatctgagtttggacggaattactccttgtccgtgtacctggtgaggcaattgactgcagggacccttctacaaaaactcagagccaaggggatccggaatccagaccattcccgggcactgatcaaggagaaactgactgctgaccccgacagtgaagtggctactacaagtctccgggtgtcactcatgtgcccgctagggaagatgcgcctgactgtcccgtgtcgtgccctcacctgtgcccatctgcagagtttcgatgctgccctttatctacagatgaatgagaagaagccgacatggacgtgtcctgtgtgtgacaagaaggctccctatgagtcgctgattattgatggtttattcatggaaattcttaattcctgttcggattgtgatgagatccagttcatggaagatggatcctggtgtccgatgaaacccaagaaggaggcatcagaggtttgccccccgccagggtatgggctggatggtctccagtacagcgcagtccaggagggaattcagccagagagtaagaagagggtcgaagtcattgacttgaccatcgaaagctcatcagatgaggaggatttgccccccaccaagaagcactgccctgtcacctcagcggccattccagcccttcctggaagcaaaggagccctgacctctggtcaccagccatcttcggtgctgcggagccctgcaatgggcacattgggcagtgatttcctgtctagtctcccgctacatgagtacccacctgccttcccactgggggctgacatccaaggtttagatttattttctttccttcagactgagagtcagcactacggcccttcagttatcacttcgctagatgaacaggacaccttgggccatttcttccagtaccgggggaccccttcccacttcctgggcccactggcccccacactggggagctctcaccgcagttccactccagcgccccctcctggtcgtgtcagcagcattgtggctcctgggagctccttgagggaagggcatggaggacccctgccttcaggtccctctttgactggctgtcggtcagacgtcatttccttggactga

SEQ ID NO: 18 - protein, 630 aa, Artificial Sequence
  Synthetic
MSVLTPLLLRGLTGSARRLPVPRAKIHSLLQVDSFRVSELQVLLGFAGRNKSGRKHELLAKALHLLKSSCAPSVQMKIKELYRRRFPRKTLGPSDLSLLSLPPGTSPPVHPDVTMKPLPFYEVYGELIRPTTLASTSSQRFEEAHFTFALTPQQLQQILTSREVLPGAKCDYTIQVQLRFCLCETSCPQEDYFPPNLFVKVNGKLCPLPGYLPPTKNGAEPKRPSRPINITPLARLSATVPNTIVVNWSSEFGRNYSLSVYLVRQLTAGTLLQKLRAKGIRNPDHSRALIKEKLTADPDSEVATTSLRVSLMCPLGKMRLTVPCRALTCAHLQSFDAALYLQMNEKKPTWTCPVCDKKAPYESLIIDGLFMEILNSCSDCDEIQFMEDGSWCPMKPKKEASEVCPPPGYGLDGLQYSAVQEGIQPESKKRVEVIDLTIESSSDEEDLPPTKKHCPVTSAAIPALPGSKGALTSGHQPSSVLRSPAMGTLGSDFLSSLPLHEYPPAFPLGADIQGLDLFSFLQTESQHYGPSVITSLDEQDTLGHFFQYRGTPSHFLGPLAPTLGSSHRSSTPAPPPGRVSSIVAPGSSLREGHGGPLPSGPSLTGCRSDVISLDAAEQKLISEEDLNGAA

SEQ ID NO: 19 - DNA, 1894 bp, Artificial Sequence
  Synthetic
atgtccgtcctgacgccgctgctgctgcggggcttgacaggctcggcccggcggctcccagtgccgcgcgccaagatccattcgttgctgcaggtcgacagtttccgagtgtctgagctccaggtgctcctcggcttcgctggcaggaacaagagtgggcggaaacacgagctgctggccaaggccctgcacctcctcaagtctagctgcgcccccagcgtccagatgaagatcaaagaactttatcgcaggcgctttccccggaagaccctggggccttctgatctctccttgctttctttgccccctggcacctctcctcctgtgcaccccgatgtcaccatgaagccactgcccttctatgaagtctatggggagctcatccgacccaccacccttgcgtccacctccagccagaggttcgaggaagcccacttcaccttcgcgctcactccccagcagctgcagcagattctcacgtccagggaagttctgccaggagccaagtgtgattacaccatacaagtgcagctcagattctgtctctgtgagaccagctgccctcaggaggactatttcccccctaacctctttgttaaggttaatgggaaactctgccccctgccgggttacctccctccaaccaagaatggagctgagcccaagaggcccagccgtccgatcaacatcacacccttggctcgactctcagccactgtccccaacaccatcgttgttaattggtcatctgagtttggacggaattactccttgtccgtgtacctggtgaggcaattgactgcagggacccttctacaaaaactcagagccaaggggatccggaatccagaccattcccgggcactgatcaaggagaaactgactgctgaccccgacagtgaagtggctactacaagtctccgggtgtcactcatgtgcccgctagggaagatgcgcctgactgtcccgtgtcgtgccctcacctgtgcccatctgcagagtttcgatgctgccctttatctacagatgaatgagaagaagccgacatggacgtgtcctgtgtgtgacaagaaggctccctatgagtcgctgattattgatggtttattcatggaaattcttaattcctgttcggattgtgatgagatccagttcatggaagatggatcctggtgtccgatgaaacccaagaaggaggcatcagaggtttgccccccgccagggtatgggctggatggtctccagtacagcgcagtccaggagggaattcagccagagagtaagaagagggtcgaagtcattgacttgaccatcgaaagctcatcagatgaggaggatttgccccccaccaagaagcactgccctgtcacctcagcggccattccagcccttcctggaagcaaaggagccctgacctctggtcaccagccatcttcggtgctgcggagccctgcaatgggcacattgggcagtgatttcctgtctagtctcccgctacatgagtacccacctgccttcccactgggggctgacatccaaggtttagatttattttctttccttcagactgagagtcagcactacggcccttcagttatcacttcgctagatgaacaggacaccttgggccatttcttccagtaccgggggaccccttcccacttcctgggcccactggcccccacactggggagctctcaccgcagttccactccagcgccccctcctggtcgtgtcagcagcattgtggctcctgggagctccttgagggaagggcatggaggacccctgccttcaggtccctctttgactggctgtcggtcagacgtcatttccttggacggccgcagaacaaaaactcatctcagaagaggatctgaatggggccgcatag

SEQ ID NO: 20 - DNA, 497 bp, Artificial Sequence
  Synthetic
cagaagcactggctgtcagaccgcacctacacctgccaggtcacctatcaaggtcacacctttgaggacagcaccaagaagtgtgcaggtacgttcccacctgccctggtggccgccacggaggccagagaagaggggcgggtgggcctcacacagccctccggtgtaccacagattccaacccgagaggggtgagcgcctacctaagccggcccagcccgttcgacctgttcatccgcaagtcgcccacgatcacctgtctggtggtggacctggcacccagcaaggggaccgtgaacctgacctggtcccgggccagtgggaagcctgtgaaccactccaccagaaaggaggagaagcagcgcaatggcacgttaaccgtcacgtccaccctgccggtgggcacccgagactggatcgagggggagacctaccagtgcagggtgacccacccccacctgcccagggccctcatgcggtccacgaccaagaccagc

SEQ ID NO: 21 - protein, 137 aa, Artificial Sequence
  Synthetic
QKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTS

SEQ ID NO: 22 - DNA, 832 bp, Artificial Sequence
  Synthetic
gtctgctccagggacttcaccccgcccaccgtgaagatcttacagtcgtcctgcgacggcggcgggcacttccccccgaccatccagctcctgtgcctcgtctctgggtacaccccagggactatcaacatcacctggctggaggacgggcaggtcatggacgtggacttgtccaccgcctctaccacgcaggagggtgagctggcctccacacaaagcgagctcaccctcagccagaagcactggctgtcagaccgcacctacacctgccaggtcacctatcaaggtcacacctttgaggacagcaccaagaagtgtgcaggtacgttcccacctgccctggtggccgccacggaggccagagaagaggggcgggtgggcctcacacagccctccggtgtaccacagattccaacccgagaggggtgagcgcctacctaagccggcccagcccgttcgacctgttcatccgcaagtcgcccacgatcacctgtctggtggtggacctggcacccagcaaggggaccgtgaacctgacctggtcccgggccagtgggaagcctgtgaaccactccaccagaaaggaggagaagcagcgcaatggcacgttaaccgtcacgtccaccctgccggtgggcacccgagactggatcgagggggagacctaccagtgcagggtgacccacccccacctgcccagggccctcatgcggtccacgaccaagaccagcggtgagccatgggcaggccggggtcgtgggggaagggagggagcgagtgagcggggcccgggctgaccccacgtctggccacaggcccgcgtgctgccccg

SEQ ID NO: 23 - protein, 221 aa, Artificial Sequence
  Synthetic
VCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAP